(19) United States
(12) Patent Application Publication
(10) Pub. No.: US 2014/0356913 A1
(43) Pub. Date: Dec. 4, 2014

(54) Enzymes Manufactured in Transgenic Soybean for Plant Biomass Engineering and Organopollutant Bioremediation

(71) Applicants: Matthew Wayne Parrow, Charlotte, NC (US); Kenneth John Piller, Davidson, NC (US); Kenneth Lee Bost, Mount Pleasant, NC (US)

(72) Inventors: Matthew Wayne Parrow, Charlotte, NC (US); Kenneth John Piller, Davidson, NC (US); Kenneth Lee Bost, Mount Pleasant, NC (US)

(21) Appl. No.: 14/229,880

(22) Filed: Mar. 29, 2014

Related U.S. Application Data
(60) Provisional application No. 61/806,502, filed on Mar. 29, 2013.

(51) Int. Cl. C12P 7/06; C12P 19/14; C12P 19/02; C12N 15/82

(52) U.S. Cl. CPC C12P 7/06; C12N 15/8255; C12P 19/14; C12P 19/02
USPC 435/99; 800/312; 435/161

(57) ABSTRACT

A strategy for eliminating or greatly reducing the need for physical/chemical treatments or the use of whole microbes for lignocellulosic biomass and organopollutant degradation is disclosed. The soybean is a practical, cost-efficient and sustainable bioreactor for the production of lignin-degrading and cellulose-degrading enzymes. The use of soybean as a transgenic overexpression platform provides advantages that no other industrial scale enzyme expression system can match. Availability of a battery of related plant biomass degrading enzymes in separate transgenic soybean lines provides unprecedented flexibility in industrial and bioremediation processes. Depending upon the particular application, selected soybean-derived powdered enzyme formulations can be used, and their sequential addition can be orchestrated. Manufacturing enzymes using transgenic soybeans wherein these enzymes are capable of lignocellulose and organopollutant degradation into useful or nontoxic products will dramatically change biomass engineering schemes and environmental remediation practices. This technology has a sum of advantages that other protein expression system cannot duplicate, including the manufacturing of individual enzymes in a cost-effective manner that allows flexibility in cocktail composition, ease of application, and long term storage in the absence of a cold chain.

The present application claims priority under 30 USC 119(e) to U.S. Provisional Patent Application No. 61/806,502 filed Mar. 29, 2013, the contents of which are incorporated by reference in its entirety.

### FIELD OF THE INVENTION

The present invention relates to the field of plant expression systems. More specifically, the present invention relates to the field of the expression of fungal enzymes in plants such as soybean. The present invention also relates to the use of transgenic soybeans to generate enzymes that are involved in the breakdown of plants and/or the production of products that result from the metabolism of plants. In one aspect, these enzymes are produced in a way that produces enzymes that are functionally active for periods longer than are currently available.

### BACKGROUND OF THE INVENTION

Three major components of lignocellulosic biomass include lignin, hemicellulose, and cellulose. Together these molecules form the matrix that is the plant cell wall, whose overall composition and intermolecular bonding can differ significantly between plant species. Since compositions and structures of plant cells walls vary, it is logical to assume that the physical methods and combination of degrading enzymes employed for efficient reduction of a particular plant species will differ, perhaps significantly.

The industrial and biotechnological applications for enzymes which can deconstruct lignocellulosic biomass are diverse and developing. Laccases have been applied to the bleaching of paper products and dyes, and for degradation of various pollutants. Lignin, manganese, and versatile peroxidases may function as additives in the food industry, or pulp lightening, or dye decolorization, or degradation of xenobiotics, or as active ingredients in cosmetic preparations. Xylanases have applications in the pulp and paper industries to facilitate bleaching, as well as scouring of fabrics. Cellulases have applications in the textile industry for modifying fabrics, in the paper industry to improve products, and even in the detergent industry to facilitate cleaning. However, the applications for enzymes that deconstruct lignocellulosic biomass which have engendered the most attention include their use in generating biofuels and improved animal feeds.

Presently there is little flexibility in commercial processes that seek to degrade lignocellulosic biomass into usable fuels or improved animal feeds. The ability to efficiently decompose lignocellulosic biomass, regardless of its plant source, will require a robust, yet easily adjustable, processing platform. At present, no such platform, theoretical or real, has been reduced to practice as an economically feasible process.

The lack of robust, yet flexible, processes to degrade lignocellulosic biomass is evidenced by the fact that of the three major components of the plant cell wall, only cellulose has routinely been targeted for commercial cellulosic ethanol production.

Standard processes in industrial plants use a variety of physical or chemical methods for lignin and hemicellulose breakdown or removal. Biorefineries typically grind or pulp biomass prior to treatments that include some combination of acids, alkali, ammonia, and/or heat to obtain fractional materials which are enriched in cellulose. Deconstruction of lignin and hemicellulose is essential to current industrial schemes solely to release or expose cellulose from the plant cell wall matrix to a sufficient extent that allows enzymatic degradation of this glucose polymer to a simple sugar. Similarly, goals for improved animal feeds concentrate cellulose-containing fractions, allowing animals to more easily digest plant materials, with the intent of increasing nutritive value. The focus on cellulosic fractions for fuels and feeds results from the inability to incorporate enzymatic degradation of lignin and hemicellulose into industrial processes in a manner that is cost-effective, efficient, and practical.

Converting lignocellulosic plant biomass into useful byproducts, such as usable biofuels and detoxifying certain polycyclic hydrocarbon organopollutants poses many challenges. Current industrial-scale degradation of plant biomass comprised of cross-linked lignin and cellulose necessitates the use of physical pretreatments, including harsh liquid-phase acid or base-catalyzed reactions. These treatments require specialized facilities for safely handling and disposing of hazardous chemicals, resulting in increased costs and environmental concerns. Likewise, current methods for enzymatic hydrolysis utilize relatively expensive purified cellulase enzymes that are applied to biomass. At present, there does not seem to be any realistic alternative to chemical and heat pretreatments since viable or killed microbial enzyme preparations are inefficient, and the expression of numerous recombinant enzymes in bulk is impracticable.

Alternative methods include use of plant biomass-degrading enzymes, which are currently primarily produced via batch culture of fungi fed substrates that induce expression of their native enzymes. The low yield/high cost of this process has impeded widespread commercial application, either by the paper industry or by theoretical cellulosic ethanol manufacturers. Industry instead primarily uses intensive chemical-physical treatments which have high energy use and pollution control requirements, and also cannot be applied in environmental remediation of aromatic organopollutants.

While numerous enzymes have been identified which can break down lignin, none are presently used in commercial processes for producing cellulosic ethanol. The enzymatic lignin degradation is limited by the recalcitrance of its aromatic backbone which requires production of a cocktail of enzymes by various prokaryotes and eukaryotes which use this material as an energy source. Laccases, peroxidases, and oxidases have identified roles in deconstructing lignin. Unfortunately, the use of such enzyme cocktails for the degradation of lignin in commercial cellulosic ethanol production remains impractical due to enzyme cost, enzyme availability, the time required for biomass reduction, and a lack of protocols which define the required quantities of specific enzyme combinations added sequentially for each particular plant species.

Like lignin, hemicellulose must be deconstructed to allow full access to polymeric cellulose. Unlike lignin, hemicellulose polymers contain various forms of the sugars xylanose, arabinose, mannose, etc. which could be directly utilized for biofuels or feeds. Cocktails of enzymes produced by various prokaryotes and eukaryotes allow utilization of this material as an energy source. Xylanses, xylosidases, endoglucanases, glucosidases, mannanases, and mannosidases have identified roles in deconstructing hemicellulose. Unfortunately, the use of such enzyme cocktails for the degradation of hemicellulose in commercial cellulosic ethanol production remains largely impractical due to enzyme cost, enzyme availability, and a lack of protocols which define the required quantities of specific enzyme combinations added sequentially for each particular plant species.

The cellulose polymer has been targeted in industrial scale biofuel and feeds as a substrate to generate glucose for fermentation. Cocktails of enzymes produced by various prokaryotes and eukaryotes have been identified which can deconstruct this polymer for use as an energy source. Endocellulases, exocellulases, and glucosidases have identified roles in degrading cellulose to glucose. For more than three decades, numerous enzymatic activities, gene sequences, and cloned enzymes from prokaryotes and eukaryotes within each of these classes have been described. Therefore it is surprising that only a few enzyme preparations are routinely utilized for industrial scale cellulosic ethanol production. Unfortunately, the use of a larger variety of enzyme cocktails for the degradation of cellulose in commercial cellulosic ethanol production remains largely impractical due to enzyme cost, enzyme availability, and a lack of protocols which define the required quantities of specific enzyme combinations added sequentially for each particular plant species.

Since current methods using chemical and enzymatic lignin/hemicellulose removal and cellulose hydrolysis are too expensive and inefficient to support commercial-scale lignocellulosic ethanol production, efforts for industrial scale lignocellulose deconstruction continue to focus on identifying platforms for manufacturing individual enzymes in a cost-effective manner that allows flexibility in cocktail composition, ease of application, and long term storage in the absence of a cold chain.

Current considerations for the physical design of biorefineries that deconstruct lignocellulose biomass for fuel must account for a source of enzymes which can degrade cellulose. Enzyme cocktails can be manufactured onsite in bioreactors or can be purchased from external commercial sources. While lignin and hemicellulose will likely be degraded by physical and chemical means at current biorefineries, the efficient reduction of released or exposed cellulose to glucose requires an enzyme cocktail. Individual or recombinant enzymes are not presently practical since they are not cost-efficient, have a limited shelf life, and often have cold-storage requirements. Due to the impracticality of using recombinant proteins, enzymes are typically produced in large bioreactors by plant-degrading fungi (e.g. Tichoderma reesei) that secrete cellulases and other enzymes during their growth. After microbial growth and enzyme induction, these cell cultures are concentrated or partially purified to provide an enzyme preparation. The shelf life for such preparations is limited with storage conditions recommended at 4-8° C.

Whether cellulase production occurs onsite, or is purchased from external commercial sources, the availability of enzyme must be temporally coupled to the lignocellulosic degradation process. Stated simply, new enzyme preparations must be available and ready for use each time a batch of lignocellulose biomass is processed. The inability of current manufacturing protocols to produce enzyme preparations with long term storage capability in the absence of a cold chain represents significant challenges for biorefinery design and significant supply chain concerns when scheduling batch deconstruction of lignocellulosic biomass.

Another challenge when designing biorefineries that deconstruct lignocellulose biomass for fuel is deciding upon the method to be used for enzymatic degradation of cellulose. Initially, separate hydrolysis and fermentation (SHF) protocols were utilized which allowed for cellulose preparations to be degraded by enzyme cocktails (e.g. cellulase plus glucosidase) in one step, followed by a separate fermentation process at a later time and under different culture conditions. Limitations of this process include end product accumulation which interferes with hydrolysis. Alternatively, during simultaneous saccharification and fermentation (SSF) cellulose preparations are added directly to fermentation tanks that already contain enzyme cocktails. Unfortunately, the reaction conditions required for these enzyme cocktails are not optimal in pH or temperature for industry-standard yeast-based fermentations, and vice versa.

A modified SSF model using filamentous fungi for both hydrolysis and fermentation has not been successful due to the low ethanol conversion and the production of unwanted acid by-products. Furthermore, SSF protocols do not allow for in situ deconstruction of lignin or hemicellulose Unfortunately, the reaction conditions required for these enzyme cocktails are not optimal for Saccharomyces cerevisiae-based fermentations, and vice versa. Furthermore, SSF protocols do not allow for in situ deconstruction of lignin or hemicellulose.

It is difficult to imagine a single reaction vessel that could efficiently achieve simultaneous ligninification, hemicellulosification, saccharification, and fermentation. For enzymatic degradation of plant cell walls, sequential processing steps using enzyme cocktails specific for lignin (e.g. laccases plus peroxidases plus oxidases), hemicellulose (e.g. xylanases plus glucanases plus mannanases), and then cellulose (e.g. endocellulases plus exocellulases plus glucosidases) will be required. While such processing steps have been theorized or explored at a laboratory scale, the practicality of sequential enzymatic processing fails due to the lack of a platform for manufacturing individual enzymes in a cost-effective manner that allows flexibility in cocktail composition, ease of application, and long term storage in the absence of a cold chain.

Presently, the logistics of continually maintaining stockpiles of various lignocellulosic degrading enzymes using current manufacturing processes and long-term storage requirements seems unlikely. To be sustainable worldwide, a platform for manufacturing cocktails of lignin- and hemicellulose- and cellulose-degrading enzymes must be flexible and practical. Ideally, such a platform would produce high levels of individual enzymes at low cost, allow formulation into customized cocktails, and be transported worldwide and stored for years prior to their use in the absence of a cold chain. Currently, there are no protein manufacturing platforms which can provide such advantages.

Natural or engineered microbes can produce or secrete cocktails of lignocellulosic degrading enzymes when grown under inducing conditions in cell culture (e.g. Trichoderma reesei). Once grown, culture fluids are harvested as enzyme preparations to be added exogenously to cellulose preparations for SHF or SSF protocols. Challenges for natural microbes producing various enzymes include the necessity to use inducing agents to express the desired enzymes, the difficulty in controlling individual enzyme ratios, and suppression of enzyme activity by end product accumulation.

Batch to batch differences in composition due variability in induction can also be problematic. For engineered organisms, constitutive promoters can overcome the induction problem. However, it is likely that a particular engineered organism must express a cocktail of enzymes (e.g. laccases plus peroxidases plus oxidases) contained within a gene cassette. Controlling the optimal ratios of each constitutively expressed enzyme needed within a cocktail will be quite difficult to accomplish in such engineered microbes. Following expression of naturally occurring or engineered enzyme cocktails in microbial cultures, the preparations are often partially purified and have to be concentrated, filtered, or lyophilized prior to use or shipping (e.g. Celluclast, Novozymes, Inc.). At this point, a limited shelf life and/or cold storage requirements add to the costs of goods and reduce the flexibility in shipping or long-term storage of enzyme preparations for future use.

An alternative platform for manufacturing lignocellulosic degrading enzymes is the use of conventional recombinant protein expression platforms in prokaryotic and eukaryotic cell cultures. Advantages of such platforms include the ability to manufacture individual enzymes, accurate quantification, and ease of formulating enzyme cocktails to degrade lignin or hemicellulose or cellulose from varying plant species biomass. Such technology is currently available, but has not been utilized on an industrial scale. Reasons for a lack of utilization can include the high cost of production and concentration, enzyme yields, limited shelf life, or the need for a cold chain. In addition, some lignocellulosic degrading enzymes have been recalcitrant to expression in standard recombinant systems. The high molecular weight, complex folding, and extensive glycosylation patterns have been given as reasons for the failure to express some functional enzymes in recombinant platforms. Other enzymes can be expressed, but are not active until refolded or are expressed at extremely low yields. Still other recombinant enzymes truncate prematurely when being expressed in some systems. In some cases, hyperglycosylation of recombinant proteins expressed in yeasts have reduced enzymatic activity. Taken together, it is difficult to imagine the sustainability of continually maintaining stockpiles of lignocellulosic degrading enzymes using current manufacturing practices whether the platform is microbial cultures or more conventional recombinant expression.

The potential for expressing recombinant industrial enzymes in plants was recognized two decades ago. Since that time, the potential for in planta expression of enzymes used in lignocellulosic degradation has been investigated. Plants which have been transformed with such enzymes include tobacco, potato, Arabidopsis, rice, corn, duckweed, alfalfa, potato, barley, and narbon bean. Recombinant enzymes have been expressed in leafy plants or in other plant tissues including seeds. Despite two decades of research and development, no industrial scale applications for plant expressed lignocellulosic degrading enzymes have currently been realized.

Efforts to identify viable, commercial scale applications have used various strategies. Initially, transgenic plants were investigated as bioreactors for the production of recombinant lignocellulosic enzymes. When active enzymes were expressed in plant tissues, problems were observed with autocatalysis affecting viability, growth, or fertility. The possibility of plant degradation or reduced viability by the very enzymes which have been introduced remains a concern. Autocatalysis of plant cell walls was addressed by expressing enzymes in organelles which sequestered active enzymes with some success. Alternatively, expression of enzymes from thermophiles that had optimal activity at high temperatures (e.g. 60° to 90° C.) was advantageous since their activity remained low at temperatures required for plant growth. Despite these advances, several limitations and uncertainties remain for the viability of in planta manufacturing of recombinant lignocellulosic enzymes.

First, some proteins do not seem to be easily expressed in some platforms. Assuming that one can target a particular enzyme to a tissue that allows viable plant growth, there still seem to be limitations in some expression systems. For example, difficulties in achieving high level expression of some full length enzymes have been reported. The inability to express some enzymes which contain a carbohydrate binding module (CBM) have resulted in the engineering of this domain from enzymes so that only fragments containing the active site are expressed. It is not altogether clear what impact such truncations will have on the effectiveness of enzymes when degrading lignocellulosic biomass from diverse plant species. While the majority of enzymes that have been expressed in plants are truncated, or are small to medium sized proteins (that is, less than about 50 kDa), many enzymes of interest are quite large (>100 kDa) or form homomers. At present, in the plants that have been used, it is not clear which platform will be most robust for such proteins which will likely be difficult to express. The ideal platform would be one which allows expression of full length enzymes that might be difficult to express in other systems, while providing an environment for protein folding into homomers if required.

Second, assuming an enzyme can be expressed in a particular plant system, it is not always clear whether that enzyme is expressed at a level sufficient for commercial viability. Difficulties in determining absolute protein expression levels stem from variability in reporting yields. In plant tissues that contain small amounts of natural protein (e.g. tobacco leaves) a high percentage of recombinant enzyme expression relative to total soluble protein looks impressive. However, the absolute yield of enzyme relative to the original plant biomass that must be harvested and processed may be modest. Furthermore, the amount of enzyme present as a percentage of total soluble protein often requires some form of partial protein purification, concentration, or other reduction of the plant biomass. Enzyme activity measurements are also difficult to compare due to differences in assays and reporting. Therefore, determining the amount, or activity, of an enzyme actually present in a given mass of harvested plant material is often difficult. Ideally, expression of high enzyme content relative to the original plant biomass would be desired.

Third, it is often not clear what level of post-harvest processing will be required to obtain a marketable enzyme or enzyme preparation. For transgenic plants expressing enzymes in tissues or organelles, some reduction in plant biomass will likely be required. Furthermore, solubilization may also be required to extract the enzyme, or to allow concentration, or to allow partial protein purification. The costs, in both materials and time, for post-harvest processing have to be considered when selecting a platform for manufacturing plant-derived lignocellulosic enzymes that are commercially viable. The need to remove excess plant tissue or concentrate enzymes prior to their utilization limits commercial viability. Ideally, little to no post-harvest processing, concentration, or purification would be required prior to marketing.

Fourth, defining intra- and inter-lot consistency in enzyme amount and/or activity will be required to obtain a marketable enzyme or enzyme preparation. Homogeneity of enzyme throughout an individual batch (intra-lot consistency) would allow proportioning of enzyme preparations for separate processes from the same batch. This may be challenging for those partially purified enzymes preparations supplied in solid form with contaminating biomass if there was no easy method to assure homogeneity prior to solubilization. Maintaining inter-lot consistency would seem more difficult for those platforms that require post-harvest processing, as consistent activity to biomass ratios would be affected by variations in concentration and/or purification methodologies. Ideally, a platform which allows easy homogenization of harvested enzyme and quantification of specific activity would permit portioning of a single lot into multiple processes or applications.

Fifth, efficient deconstruction of lignocellulosic biomass requires cocktails of enzymes that are active in the appropriate proportions and at the correct time in the reaction mixture. Furthermore, depending on the species of plant biomass to be degraded, such reactions will have different enzyme compositions and conditions. The availability of formulations or compositions of individual enzymes which could be added in the correct quantity and at the appropriate time would allow great flexibility in manufacturing cellulosic ethanol or feeds from a diversity of biomass species. For such compositions to be formulated, a method for straightforward and quantitative mixing of individual enzyme preparations into unique combinations would be required. Presently, no such protocols for easily constructing customizable lignocellulosic enzyme compositions exist.

Sixth, perhaps one of the most important features of a platform technology for plant-derived lignocellulosic enzymes is stability of storage over time using ambient storage conditions. Often the stability of a particular plant-derived enzyme preparation is reported in the context of a particular condition (e.g. heat, pH, etc.) or a particular application (e.g. activity over minutes to hours for degrading cellulose substrates). However the long term stability of a stored enzyme preparation is rarely demonstrated. The ability to store enzyme preparations for years to decades in the absence of a cold chain and without a significant loss of activity cannot currently be achieved. If such a platform technology could be discovered, it would have profound implications for the future design of biorefineries, supply chain logistics, and manufacturing processes. The ability to produce individual enzymes in plant lines and store the harvested product for multiple years in ambient conditions would allow these enzymes to be manufactured anywhere in the world. The ability to transport these stable enzymes using conventional shipping to any biorefinery in the absence of a cold chain would eliminate the need for onsite enzyme production facilities and/or transport refrigeration. Furthermore, coordinating pretreatment of lignocellulosic biomass, with the near-simultaneous production or acquisition of enzymes that have a limited shelf life, would no longer be required. Such flexibility in the manufacturing process would be a significant advantage that presently does not exist. Unfortunately, no current platform technology can produce enzymes in a form that is stable long-term at ambient storage temperatures.

Seventh, a platform technology for in planta expression of individual lignocellulosic degrading enzymes will not be commercially viable if it is not cost-effective. To date, no platform has demonstrated such viability for generating cellulosic ethanol.

In addition to using transgenic plants as bioreactors to produce enzymes, other strategies have also been proposed. For example, plant crops have been engineered to express selected degrading enzymes as a value added trait. Upon harvest of these engineered crops, autocatalysis would permit more efficient deconstruction of their lignocellulose biomass in various industrial applications. Theoretically, such crops could be used for cellulosic ethanol production or to make feedstocks more digestible. The proposal to establish genetically modified crops to be grown in mass quantities would, theoretically, provide biomass that would be more amenable to deconstruction. However, this solution does not address lignocellulosic degradation of any non-genetically modified plant biomass.

Moreover, only certain fungi have enzymes and the appropriate machinery to degrade lignin and cellulose. These fungi include: (1) Brown-rot fungi break down hemicelluloses and cellulose; examples include Serpula lacrymans, Fibroporia viallantii, (oniophor puleana, Phaeolus schweinilzii and fomitopsis pinicola; (2) Soft-rot fungi secrete cellulases from their hyphae; examples include Chactomium, Ceralocstis, and Kretzchmaria; (3) White-rot fungi generally degrade lignin, with some species also capable of degrading cellulose; examples include Pleurotus ostreatus, Phanerochaete chyrsosporium and Ceripriporiopsis subvermispora; Trichoderma is a genus of fungi that are culturable. The cellulose degrading enzymes derived from species such as T. reesei and T. viride have drawn recent attention. The lignin degrading enzymes expressed by fungi are grouped into four major categories referred to as lipid peroxidases (LiPs), manganese peroxidases (MnPs), Versatile peroxidases (VPs) and laccases. Cellulose and hemicelluloses degrading enzymes are also grouped into broad categories, referred to as endocellulases, exocellulases, cellobioases, oxidative cellulases, and cellulose phosphorylases. Within all of the above categories, there can be isoforms. To date, some of the above enzymes have been overexpressed and then used as either crude extracts or purified.

It is difficult to express recombinant forms of these enzymes in traditional systems (e.g. E. coli, yeast, mammalian cell cultures, etc.) for a variety of reasons, including improper folding, a requirement for cofactors (e.g. manganese, heme), and associated production and/or purification costs that are not practical and sustainable for industrial applications. Plants typically are not considered as bioreactors for these plant degrading enzymes due to the presence of cellulose and lignin that are required as structural components of plants. While fungi produce these important enzymes, many of the species are not culturable. Furthermore, the level of protein in fungi is relatively low, making it a less than ideal bioreactor for the production of enzymatic proteins.

The word “incremental” best describes recent advances that have been made when tackling the problem of converting lignocellulosic biomass into useful byproducts (e.g. glucose) and for detoxifying lignin-like aromatic organopollutants. Industrial methods have focused on caustic, energy-intensive treatments using chemicals, heat, and pressure. Biological methods have focused on mass production of microbes such as fungi and their native enzymes. Unfortunately, these technologies are expensive and have application limits that hinder industry. Efficient industrial-scale degradation of biomass comprised of cross-linked lignin and cellulose currently necessitates the use of physical pretreatments. At present, there does not seem to be any realistic alternative to chemical and heat pretreatments since viable or killed microbial enzyme preparations are inefficient, and the expression of numerous recombinant enzymes in bulk is impracticable. While incremental steps are being made to overcome these limitations, it is unclear if such small advances will be sufficient to make pragmatic changes in current biomass processing. These drawbacks are primary factors inhibiting the sustainability of industries such as lignocellulosic ethanol production. In fact, even with significant research in acid and isolated enzyme hydrolysis, there is currently no significant industrial production of lignocellulosic ethanol in the United States. Essentially, current production techniques are too expensive to support commercial interests and it appears that, despite enormous potential, lignocellulose bioprocessing will remain underutilized unless new processing technologies that are feasible and economical are developed. To propose a wholly enzymatic strategy for biomass degradation would require transforming technologies.

### BRIEF SUMMARY OF THE INVENTION

In an embodiment, the present invention relates to a platform technology for expressing lignin-cellulose degrading enzymes in transgenic soybean seeds. This technology has a sum of advantages that other protein expression system cannot duplicate, including the manufacturing of individual enzymes in a cost-effective manner that allows flexibility in cocktail composition, ease of application, and long term storage in the absence of a cold chain.

However, the innovation in this invention does not end with the advantages of the novel protein expression system.

Accordingly, in an embodiment, the present invention relates to a strategy for eliminating (or greatly reducing) the need for physical/chemical treatments or the use of whole microbes for lignocellulosic biomass and organopollutant degradation. In one embodiment, the present invention relates to the use of a soybean as a practical, cost-efficient and sustainable bioreactor for the production of lignin-degrading and cellulose-degrading enzymes. The use of soybean as a transgenic overexpression platform should provide advantages that no other industrial scale enzyme expression system can match.

Thus, in several embodiments of the present invention, this invention includes:
1) newly designed genes for expressing enzymes aimed at bioremediation in soybean seeds (a composition of matter);
2) stable expression of plant biomass/aromatic organopollutant degrading enzymes of fungal origin (e.g. ligninases, laccases, cellulases) in a plant tissue (i.e. soybean seeds) that can be propagated as continuous lines (a composition of matter);
3) manufacturing commercial scale quantities of said enzyme proteins targeted for expression in the soybean seed (new process);
4) the processing and/or formulation of transgenic soybean seeds expressing fungal biomass-degrading enzymes into powders or liquids for long-term storage in the absence of a cold chain (new process);
5) the formulation of transgenic soybean seeds expressing lignocellulose degrading/bioremediation enzymes into powders or liquids for applications (e.g. lignocellulosic or organopollutant bioremediation) (new process);
6) processes for sequential or orchestrated treatment of plant biomass and aromatic organopollutants using these enzymes produced in transgenic soybean seeds (new process);
7) infrastructure or devices which support applications for plant biomass/aromatic organopollutants-degrading enzymes produced in transgenic soybean seeds (new devices).

Thus, in an embodiment, the present invention also relates to a strategy for eliminating (or greatly reducing) the need for physical and chemical pretreatments or the use of microbes for biomass and toxin degradation. Expressing lignin-cellulose degrading enzymes in transgenic soybean seeds will provide advantages that no other industrial scale protein expression system can match.

However, the innovation in this invention does not end with the advantages of this novel protein expression system. Availability of a variety of plant biomass degrading enzymes in separate transgenic soybean lines would provide unprecedented flexibility in bioremediation processes. Depending upon the particular application, selected soybean-derived enzyme formulations could be used, and their sequential addition could be orchestrated. Stated simply, availability of easily manufactured enzymes capable of biomass and toxin deconstruction could dramatically change industrial processing schemes and infrastructure, as well as environmental remediation efforts.

### BRIEF DESCRIPTION OF THE SEVERAL VIEWS OF THE DRAWING

FIG. 1 shows immunofluorescence showing subcellular localization of different heterologous proteins (solid arrows) in soybean seeds.

FIG. 2 shows a Western blot showing heterologous FanC protein in intact soybeans and ground seed powder stored for 8 years under ambient conditions.

FIG. 3 shows Western blots showing solubility of heterologous protein in seed powder compositions comprising particles with known diameters.

FIG. 4 shows quantification of bulk soy protein solubilized in seed powder compositions with particles of known size.

FIG. 5 shows Western blots showing dissolution of protein in compositions comprising multiple heterologous proteins.

FIGS. 6A-E show the amino acid sequences (in one letter code) for Laccases from various species (see Table 1 to ascertain which species corresponds to which sequence).

FIGS. 7A-D show the amino acid sequences (in one letter code) for Lignin Peroxidases from various species (see Table 1 to ascertain which species corresponds to which sequence).

FIGS. 8A-D show the amino acid sequences (in one letter code) for Manganese Peroxidases from various species (see Table 1 to ascertain which species corresponds to which sequence).

FIGS. 9A-C show the amino acid sequences (in one letter code) for Versatile Peroxidases from various species (see Table 1 to ascertain which species corresponds to which sequence).

FIGS. 10A-C show the amino acid sequences (in one letter code) for Aryl Alcohol Oxidases from various species (see Table 1 to ascertain which species corresponds to which sequence).

FIGS. 11A-E show the amino acid sequences (in one letter code) for Xylanases from various species (see Table 2 to ascertain which species corresponds to which sequence).

FIGS. 12A-C show the amino acid sequences (in one letter code) for Xylan Xylosidases from various species (see Table 2 to ascertain which species corresponds to which sequence).

FIGS. 13A-B show the amino acid sequences (in one letter code) for Xyloglucan specific b 1,4 endoglucanases from various species (see Table 2 to ascertain which species corresponds to which sequence).

FIGS. 14A-B show the amino acid sequences (in one letter code) for Glucan b 1,4 glucosidases from various species (see Table 2 to ascertain which species corresponds to which sequence).

FIGS. 15A-B show the amino acid sequences (in one letter code) for B 1,4 endomannanases from various species (see Table 2 to ascertain which species corresponds to which sequence).

FIG. 16A shows the amino acid sequence (in one letter code) for a B 1,4 mannosidase from a particular species (see Table 2 to ascertain the species).

FIGS. 17A-F show the amino acid sequences (in one letter code) for Endocellulases from various species (see Table 3 to ascertain which species corresponds to which sequence).

FIGS. 18A-C show the amino acid sequences (in one letter code) for Exocellulases from various species (see Table 3 to ascertain which species corresponds to which sequence).

FIGS. 19A-C show the amino acid sequences (in one letter code) for b-Glucosidases from various species (see Table 3 to ascertain which species corresponds to which sequence).

FIG. 20A-E show the nucleic acid sequences for a synthetic fanC sequence designed using the codon table 4 and four different mSEB variants designed based upon the procedures discussed below.

FIG. 21 shows the purification of heterologous hTg 660 kDalton homodimeric protein expressed in soybean seeds.

FIGS. 22A-D show four peptides from Tables 1, 2 and 3 that have been queried in a signal prediction programs (one from each of Tables 1, 2 and 3 (FIGS. 22A, 22B, and 22D, respectively)) and a second one from table 3 that contains no signal peptide sequence (FIG. 22C). The full sequences are shown and the signal peptides are underlined.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New methods and systems to express fungal enzymes in plants such as soybean are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

This invention embodies the design of synthetic cellulose degrading and lignin degrading genes derived from the categories of enzymes such as endocellulases, exocellulases, cellobioases, oxidative cellulases, and cellulose phosphorylases for the preferential expression and accumulation in soybean seeds. The use of soy as a host for expression of plant-degrading enzymes is not obvious because soy is a plant (and these enzymes naturally metabolize plants). Heterologous expression of fungal LiP, MnP, VP, laccase, and the suite of cellulases will have applications in ligno-cellulosic processing and organopollutant bioremediation. These synthetic genes can be optimized for expression in soybean seeds (e.g. via codon usage, GC content, removal of cryptic regulatory sequences, etc.) and contain regulatory elements (e.g. leader peptides, transit peptides, retention signals, targeting peptides, tags for purification, etc.) to target expression to specific locations within the soybean seed (e.g. the E.R., protein storage vesicles, protein storage bodies, cytosol, etc.) or aid in purification. Seed specific promoters are used to accumulate these products during seed development, especially late seed development. Such targeting will be accomplished with standard glycinin (e.g. 11S) and conglycinins (e.g. 7S) seed-specific promoters. Synthetic gene cassettes can be transferred to soybean using a variety of methods (e.g. Agrobacterium-mediated transformation, electroporation, etc.).

The expression of plant degrading enzymes accumulates as a component of the seed storage reserve protein, and should not be detrimental to survival of the plant. The availability of endogenous cofactors (e.g. manganese, heme) within the seed supports the synthesis of functional proteins, which are problematic with recombinant expression of such proteins in yeast or E. coli. The high protein content of soybeans, the presence of necessary cofactors, and the ability to sequester heterologous enzymes from endogenous lignin and cellulose supports the notion that the present invention will be a transformative technology. Accordingly, the present invention relates to the production of massive amounts of these lingo-cellulosic proteins that can be produced at practical costs, providing unprecedented advantages for their use in industrial applications.

In addition to applications for biofuel production (e.g. conversion to ethanol), the enzymes derived from some fungi have properties that can detoxify organopollutants, and thus have uses in bioremediation. For example, the enzymes present in white rot fungus degrade polyaromatic hydrocarbons (PAHs), chlorinated aromatic hydrocarbons (CAHs), polycyclic aromatics, polychlorinated biphenyls, polychlorinated dibenzo(p)dioxins, the pesticides DDT and lindane, and some azo dyes. Accordingly, overexpressing these enzymes in massive amounts will allow the production of proteins at practical costs, and provide unprecedented advantages for their use in industrial pollution control applications.

To the inventors' knowledge, the expression of recombinant enzymes capable of degrading lignocellulosic biomass in transgenic soybean seeds has not been reduced to practice nor has adequate description appeared that would allow one to make and use enzymes derived from transgenic soy without undue experimentation. This is surprising since the platform for recombinant protein expression has demonstrated some unique and unexpected advantages. Taken together, the sum of these unique features of transgenic soybeans represents a platform that no other protein expression system can achieve.

First, targeting of enzyme expression to the soybean seed minimizes the deleterious effects that such degrading enzymes might have on plant growth, maturation, and seeding. The ability of the soybean seed to allow protein packaging amongst soy seed proteins would also limit in vivo enzymatic activity. Therefore enzymes which cannot be expressed in other plant systems due to toxicity or lethality will likely be expressed in this system.

Second, exogenous protein expressed in transgenic soybean seeds achieves some of the highest recombinant protein to raw biomass ratio of any plant expression system. For example, expression levels as high as 13 grams of recombinant protein per liter of harvested soybean seeds has been achieved. These quantities exceed current industry values for enzyme to biomass ratios at harvest prior to any concentrations, purifications, filtrations, or lyophilizations for any of the other plant expression (usually by an order of magnitude or larger).

Third, due to the high ratio of recombinant protein to soybean biomass, no purification, concentration, lyophilization, or filtration is required for applications. Powders and combinations of powders made from transgenic soybean seeds can be added directly to lignocellulosic degrading processes or to agricultural feedstocks without any additional processing. Furthermore, powders can be homogenized, allowing intra-lot consistency. This permits proportioning of enzyme preparations so that the same batch might be used for separate processes. Powders from individual transgenic soybean lines expressing particular lignocellulosic enzymes could be formulated into customizable cocktails containing the desired quantities and ratios of each enzyme desired.

Furthermore, the ability to grind soybean seeds expressing enzymes to a relatively uniform particle sized powder (5-200 micrometers or alternatively, 5 to 1600 micrometers) allows variability in dissolution upon addition to a particular processing step. Ability to subject soybean powders to additional processing that is standard for the industry (e.g. hexane treatment to remove oils; alcohol treatment to remove carbohydrates, heating to make biofeeds edible, etc.), while maintaining enzymatic activity in soy flakes or powders, prior to use or storage also adds flexibility for particular applications.

Fourth, transgenic soybean seeds are capable of expressing recombinant proteins that are difficult or impossible to express in other protein expression systems. The ability to glycosylate, fold, homomerize, and add prosthetic groups (e.g. metalloproteins) permits a variety of functional enzymes to be manufactured that are not easily performed in other expression systems.

Fifth, the natural ability of soybean seeds to express and package proteins in a heat-stable and desiccant-resistant environment allows for long-term storage. The ability to store transgenic seeds, soy powders, or soy formulations expressing a recombinant enzyme for many years or decades in the absence of a cold chain permits separating protein expression from its use. Stated simply, the manufacture of individual degrading enzymes can occur offsite, prior to their transportation, long term storage, and eventual use at facilities around the world. The ability to store powders for >12 months (or 2 years or longer) in the absence of a cold chain without substantial loss of enzyme activity allows stockpiling of enzymes and flexibility in processing lignocellulosic biomass.

Sixth, platform technologies for manufacturing lignocellulosic degrading enzymes must be cost-efficient. This is one of the most significant limitations for producing individual enzymes to be used in sequential, step-wise degradation. Since transgenic soybean seeds are efficient at producing, concentrating, and storing proteins, these characteristics allow some of the lowest costs per milligram per biomass of any industrial platform. The ability to manufacture recombinant enzymes at fractions of a cent per milligram puts transgenic soybean seeds as one of the most cost-efficient approaches.

Thus, in an embodiment, soybean-derived enzymes solve the following problems and/or provide the following advantages:
1) ability to successfully express protein degrading enzymes in high concentration in soybean seed without degrading the seed to a point where it is non-germinable.
2) ability to express such enzymes in high concentration that are difficult or impractical to express in other plant-derived systems
2) dramatically reducing cost of production of enzymes
3) provide the highest biomass to enzyme ratios of any plant-derived system
3) dramatically simplify the harvesting of enzymes and their formulation for use
4) ability to store soy-powder expressing enzymes for extended periods of time without cold chain prior to their use.
5) unique formulations of soy-derived enzymes for bioremediation e.g. powder
6) long term stability in such unique formulations
7) unique sequential processes that result from having individual soybean lines expressing particular enzymes (e.g. treat with ligninase, then cellulose, then lipase, etc.)
8) infrastructure or devices which support soy powder-based enzymatic treatment (e.g. a canister or cartridge filled with soy powder expressing an enzyme that can degrade an environmental pollutant, and then passing the pollutant over it to detoxify).

In an embodiment, the present invention relates to transgenic soybeans that express enzymes being capable of at least partially metabolizing cellulose, lignin, and/or hemicellulose.

Examples of these enzymes that have the ability to at least partially metabolize cellulose, lignin, and/or hemicellulose appear in Tables 1-3 below. The protein sequences can be used to synthesize the ideal cDNA that can be overexpressed in a transgenic soy plant as described in detail below.

Table 1 shows lignin deconstruction enzyme examples.

TABLE 1
Lignin deconstruction enzyme examples

| | Organism | FIGURE SEQ ID NO |
|---|---|---|
| Laccases | | |
| LLC1 | Trametes versicolor | 6A—SEQ ID NO: 1 |
| MtL | Myceliophthora | 6B—SEQ ID NO: 2 |
| | thermophila | |
| ERY4 | Pleurotus eryngii | 6C—SEQ ID NO: 3 |
| Lac1 | Pycnoporus cinnabarinus | 6D—SEQ ID NO: 4 |
| Lac | Thermus thermophilus | 6E—SEQ ID NO: 5 |
| Lignin Peroxidases | | |
| CiP | Coprinus cinereus | 7A—SEQ ID NO: 6 |
| LiPH8 | Phanerochaete | 7B—SEQ ID NO: 7 |
| | chrysosporium | |
| LiP | Trametes cervina | 7C—SEQ ID NO: 8 |
| PrLiP1, PrLip4, PrLip3 | Phlebia radiata | 7D—SEQ ID NO: 9 |
| Manganese Peroxidases | | |
| MnP | Phanerochaete | 8A—SEQ ID NO: 10 |
| | chrysosporidium | |
| MnP Isoform 1 | Dichomitus squalens | 8B—SEQ ID NO: 11 |
| MnP Isoform 2 | Dichomitus squalens | 8C—SEQ ID NO: 12 |
| MnP isoenzyme | Pleurotus ostreatus | 8D—SEQ ID NO: 13 |
| Versatile Peroxidases | | |
| VP | Pleurotus ostreatus | 9A—SEQ ID NO: 14 |
| ViP | Bjerkandera adusta | 9B—SEQ ID NO: 15 |
| ViP | Pleurotus eryngii | 9C—SEQ ID NO: 16 |
| Aryl Alcohol Oxidase | | |
| AAO | Pleurotus pulmonarius | 10A—SEQ ID NO: 17 |
| Oxidase | Aspergillus terreus | 10B—SEQ ID NO: 18 |
| Oxidase | Pleurotus eryngii | 10C—SEQ ID NO: 19 |

Table 2 shows examples of hemicellulose deconstruction enzymes.

TABLE 2
Hemicellulose deconstruction enzyme examples

| | Organism | FIGURE SEQ ID NO |
|---|---|---|
| Xylanases | | |
| EGL | Talaromyces emmersonii | 11A—SEQ ID NO: 20 |
| xynb | Aspergillis niger | 11B—SEQ ID NO: 21 |
| MEY-1 | Bispora sp. | 11C—SEQ ID NO: 22 |
| Xyn | Streptomyces spS27 | 11D—SEQ ID NO: 23 |
| XynA | Bacillus | 11E—SEQ ID NO: 24 |
| Xylan Xylosidases | | |
| B XTE | Talaromyces emmersonii | 12A—SEQ ID NO: 25 |
| XylC | Thermoanaerobacterium | 12B—SEQ ID NO: 26 |
| | saccharolyticum | |
| XlnD | Aspegillis niger | 12C—SEQ ID NO: 27 |
| Xyloglucan specific | | |
| b 1,4 endoglucanase | | |
| egl | Aspergillus aculeatus | 13A—SEQ ID NO: 28 |
| eglC | Aspergillis niger | 13B—SEQ ID NO: 29 |
| Glucan b 1,4 | | |
| glucosidase | | |
| glucosidase | Irpex lacteus | 14A—SEQ ID NO. 30 |
| Cel48A | Thermobifida fusca | 14B—SEQ ID NO: 31 |
| B 1,4 endomannanase | | |
| mannanase | Biospora sp. | 15A—SEQ ID NO: 32 |
| endo-beta-1,4- | Aspergillus fumigatus | 15B—SEO ID NO: 33 |
| mannosidase | | |
| B 1,4 mannosidase | | |
| manB | Aspergillus aculeatus | 16A—SEQ ID NO: 34 |

Table 3 shows examples of cellulose deconstruction enzymes.

TABLE 3
Examples of cellulose deconstruction enzymes

| Enzyme Class | Organism | FIGURE SEQ ID NO |
|---|---|---|
| Endocellulase | Anaerocellum thermophilum | 17A—SEQ ID NO: 35 |
| CelA | (now Caldicellulosiruptor bescii) | |
| CelA | Caldocellum saccharolyticum | 17B—SEQ ID NO: 36 |
| CelA | Thermotoga neapolitana | 17C—SEQ ID NO: 37 |
| CelB | Thermotoga neapolitana | 17D—SEQ ID NO: 38 |
| EglA | Pyrococcus furiosus | 17E—SEQ ID NO: 39 |
| CelA | Rhodothermus marinus | 17F—SEQ ID NO: 40 |
| Exocellulase | Streptomyces sp. M23 | 18A—SEQ ID NO: 41 |
| Cel7A | Thermoascus aurantiacus | 18B—SEQ ID NO: 42 |
| CelO | Clostridium thermocellum | 18C—SEQ ID NO: 43 |
| b-Glucosidase | Sporotrichum thermophile | 19A—SEQ ID NO: 44 |
| glucohydrolase, | (Synonym Myceliophthora | |
| B-glucosidase | thermophila) | |
| B-glucosidase | Periconia sp | 19B—SEQ ID NO: 45 |
| B-glucosidase | Volvariella volvacea | 19C—SEQ ID NO: 46 |

### Experimental Detail and Protocols

### How to Make Synthetic Genes

To obtain optimal expression of a gene and protein product in a heterologous system it is important that the foreign gene is recognized by the host expression system as an “expressable gene”. Many biological systems exhibit trends in structure and content which differentiate them from other systems. Two examples of characteristics that can affect heterologous gene expression are (1) GC content (or conversely AT content) of nucleotides present in genes, chromatin and genomes, and (2) the use of “preferred” codons by different systems during protein synthesis.

To expand upon the point regarding GC content, attention can be drawn to the genomes of three bacteria from a study sequenced in 2004. In that study it was reported that the AT content of Bdellovibrio bacteriovrus was 49.4% of the genome while the AT content of Lactobacillus johnsonii and Mycoplasma mycoides represented 65.4 and 76 percent of the nucleotides in the genome, respectively. The GC content of different organism genes and genomes are identified as more sequence data is obtained from EST and genome sequencing. Simple internet searches using the terms can relatively quickly reveal the GC content of a species. A useful scientific website for obtaining information on GC content of various genomes can be found at http:://www.ncbi.nlm.nih.gov/genome/. Using “Glycine max” (soybean) as the search criteria with the above website reveals a 35% GC content for soybean. It is generally understood that the GC content of some examples include Plasmodium falciparum at ˜20%, Arabidopsis thaliana at ˜36%; Saccaromyces cerivisea at ˜38% and Homo sapiens at ˜40%. Genetic engineers have used GC content information when designing genes for expression in heterologous systems. For example, a Plasmodium falciparum gene with a 20% GC content may not express well in soybean which has a GC content of 35%. For a reason such as this that same Plasmodium gene may be engineered with a GC content of ˜35% so that the synthetic gene appears more similar to genes expressed in soybean. If time and cost permit, multiple genes such as a native sequence and an engineered sequence can be tested alongside each other. If only one gene sequence can be tested, genetic engineers will often choose to design and synthesize a gene with a GC content that is similar to the GC content of genes expressed in the host expression system. In the case of soybean, the present invention will engineer enzymes for deconstruction of lignin, hemicellulose and cellulose degradation with GC content that are similar to those of endogenous soybean genes.

Regarding codon usage, it is important to note that the term “codon usage” or “codon bias” refers to differences in synonymous codons that can be used during protein translation. There are 64 different codons that comprise the 20 amino acids (and selenocysteine) used in protein synthesis; 61 of the codons encode the various 20 amino acids while three codons function as termination codons (one of which periodically codes for selenocysteine). Thus, multiple codons are used to encode the same amino acid. For example, the codons UUA, UUG, CUU, CUC, CUA, and CUG all encode the amino acid leucine. Different organisms use the various codons in different preferred ratios or biases. The codon preferences or biases of different organisms can be summarized from the analysis of one or more genes and then assembled into tables referred to as codon bias tables or codon preference tables. To highlight the codon biases of different organisms, a comparison of E. coli and plant codon tables will reveal that of the six codon encoding leucine, CUG is the preferred codon for leucine in E. coli proteins while CUU and CUC are preferred codons for leucine in plant proteins. Thus, a gene engineered for expression in soybean will preferentially use the gene sequences CTT and CTC more frequently than the other four sequences when leucine is to be encoded. Note that the use of CTC can increase the GC content of a synthetic gene while the use of CTT can decrease the GC content of the gene. Thus, the specific choices of codon usage can not only create a synthetic gene with codon biases similar to the host organism, but can also be used to manipulate the GC content of a synthetic gene so that its overall GC content is comparable to the GC content of the host organism. When coding for a particular amino acid in the enzymes that partially degrade cellulose, lignin and/or hemicellulose, this methodology will be used.

Codon bias tables can be compiled in many different ways. For example, a soybean codon table can be generated from data obtained from every gene expressed in soybean. Alternatively, such a table can be assembled for genes expressed only in soybean seeds. Alternatively, such a table can be made for genes expressed at a particular stage of soybean seed development. Alternatively, such a table can be made that is representative of the glycinin family of genes expressed in seed development. Thus, codon bias tables can be customized to meet many different needs, and can be generated from the analysis of as little as one protein sequence to as many as thousands of sequences or more. This customization will be used in the present invention when coding for a particular amino acid in the enzymes that partially degrade cellulose, lignin and/or hemicellulose.

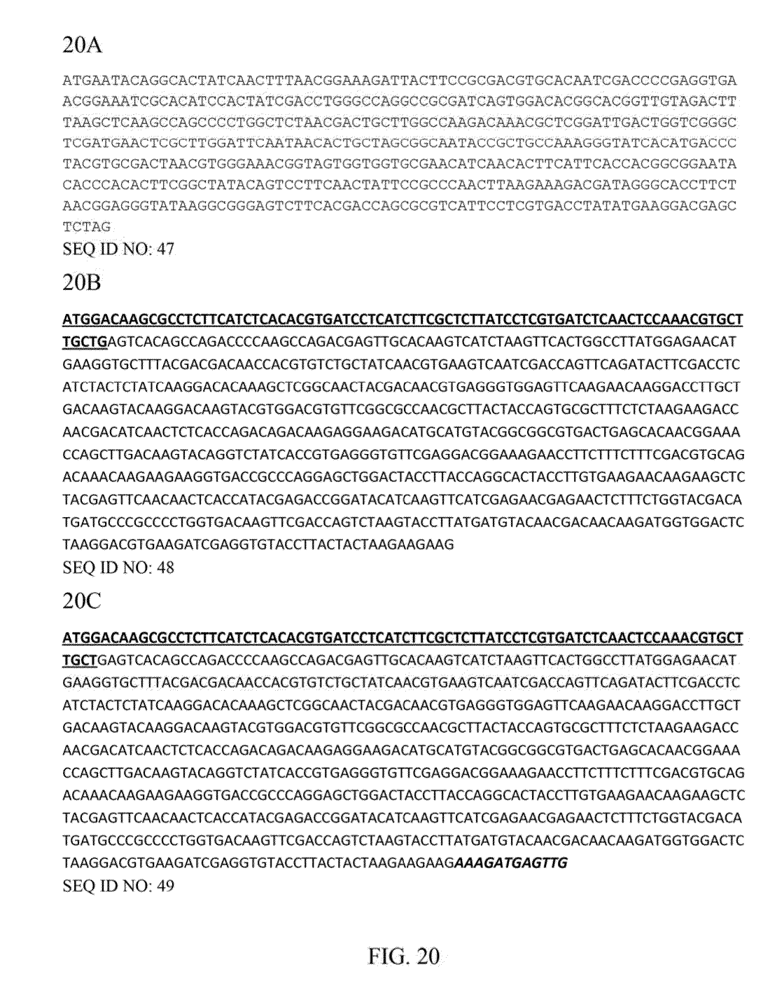

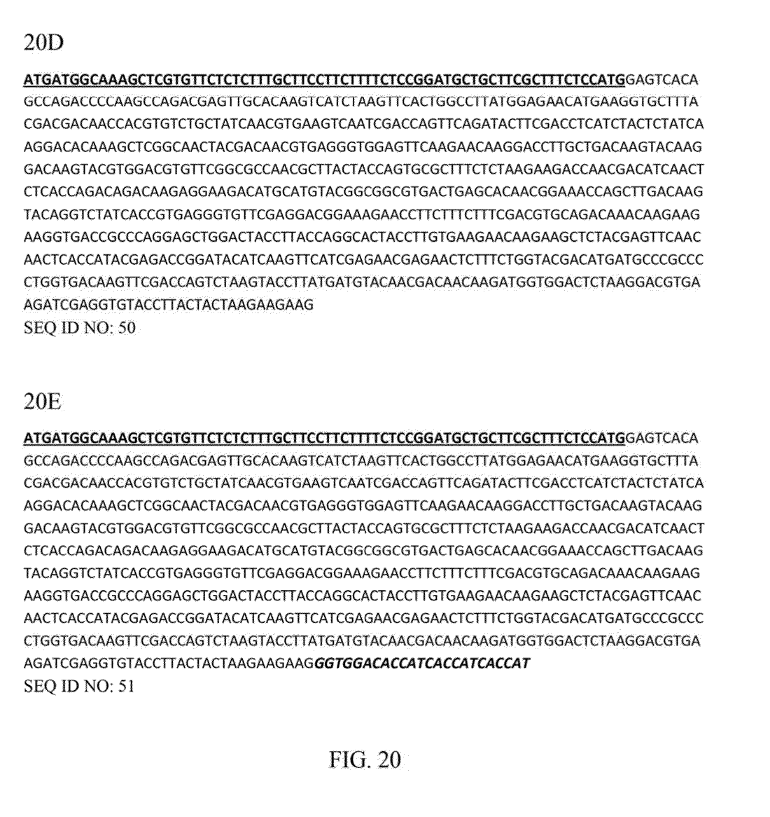

If expression of a heterologous gene is desired to accumulate in soybean seeds, then a genetic engineer may choose to utilize a seed-specific promoter to drive expression spatially and temporally. Some common seed-specific promoters used for seed-specific expression of heterologous genes include the glycinin (termed 11S proteins based on sedimentation) and conglycinin (termed 7S) promoters. Glycinins and conglycinins are abundant seed storage proteins that accumulate in the developing seed. Thus, a soybean 7S or 11S promoter may be chosen to drive expression of a heterologous deconstruction enzyme such as endocellulase as accumulation of the heterologous gene would have the same accumulation profile as the native 7S or 11S storage proteins. A genetic engineer may therefore choose to create a codon bias table based on known soybean glycinin sequences or conglycinin sequences and in turn use that customized codon bias table to engineer the desired heterologous gene (e.g. endocellulase or any other deconstruction gene). Alternatively, the genetic engineer may choose to create a codon bias table based on known glycinin plus conglycinin protein sequences since these two families represent the majority of the storage proteins present in soybean seeds. An example of a codon bias table for soybean seed storage proteins is shown below in Table 4. This table was generated by obtaining eight different soybean glycinin and conglycinin protein sequences, stringing these sequences together into one long concatamer and then determining the frequency of each codon used. This customized soybean seed codon bias table can then be used with a variety of algorithms to generate a synthetic DNA sequences that can be used to encode a specific gene intended for expression and accumulation within soybean seeds. Table 4 below was also used to generate a synthetic FanC gene which was stably integrated into the soybean genome and successfully expressed and accumulated FanC protein (see FIGS. 20A-E).

TABLE 4
Glycine max seed storage protein concatamer (8 proteins) (4222 codons)
fields: [triplet] [frequency: per thousand] ([number])

| | | | | | | | | | | | |
|---|---|---|---|---|---|---|---|---|---|---|---|
| UUU | 15.6 | (66) | UCU | 14.4 | (61) | UAU | 9.2 | (39) | UGU | 3.8 | (16) |
| UUC | 31.7 | (134) | UCC | 10.4 | (44) | UAC | 15.4 | (65) | UGC | 10.4 | (44) |
| UUA | 3.3 | (14) | UCA | 12.1 | (51) | UAA | 0.0 | (0) | UGA | 0.0 | (0) |
| UUG | 17.8 | (75) | UCG | 3.3 | (14) | UAG | 0.0 | (0) | UGG | 6.2 | (26) |
| CUU | 21.8 | (92) | CCU | 23.4 | (99) | CAU | 6.2 | (26) | CGU | 8.1 | (34) |
| CUC | 21.6 | (91) | CCC | 11.6 | (49) | CAC | 14.7 | (62) | CGC | 13.3 | (56) |
| CUA | 7.3 | (31) | CCA | 23.2 | (98) | CAA | 50.0 | (211) | CGA | 6.2 | (26) |
| CUG | 9.9 | (42) | CCG | 3.1 | (13) | CAG | 40.5 | (171) | CGG | 4.5 | (19) |
| AUU | 20.4 | (86) | ACU | 7.9 | (33) | AAU | 17.1 | (72) | AGU | 13.7 | (58) |
| AUC | 13.7 | (58) | ACC | 14.9 | (63) | AAC | 51.9 | (219) | AGC | 21.1 | (89) |
| AUA | 12.1 | (51) | ACA | 6.9 | (29) | AAA | 20.8 | (88) | AGA | 21.6 | (91) |
| AUG | 8.8 | (37) | ACG | 1.4 | (6) | AAG | 28.9 | (122) | AGG | 12.8 | (54) |
| GUU | 17.3 | (73) | GCU | 16.1 | (68) | GAU | 19.9 | (84) | GGU | 19.4 | (82) |
| GUC | 6.6 | (28) | GCC | 16.1 | (68) | GAC | 23.2 | (98) | GGC | 11.1 | (47) |
| GUA | 4.3 | (18) | GCA | 14.4 | (61) | GAA | 47.8 | (202) | GGA | 21.8 | (92) |
| GUG | 26.5 | (112) | GCG | 4.0 | (17) | GAG | 49.5 | (209) | GGG | 9.0 | (38) |

### Assembling a Gene Using Lone Overlapping Primers

There are several ways to assemble a synthetic gene once it is designed. One method involves the synthesis of long (e.g. 100-150 bp) primers which span the entire region of both strands of a gene sequence. These long primers are designed such that they each contain long overlapping ends (e.g. 15-30 bp) which allows the primers to be annealed in pairwise manner. Raising the temperature of a solution and then lowering the temperature will allow complementary overlapping primers to anneal. Alternatively, many long overlapping primers can be annealed simultaneously. Following an annealing reaction, enzymes such as T4 DNA ligase can be used to join primers together. The joined strands can serve as a template for subsequent DNA amplification utilizing the polymerase chain reaction (PCR) method. For PCR amplification, the 5′-most (forward) oligonucleotide primer and 3′-most (reverse) oligonucleotide primer from the original annealing reaction can be utilized. Alternatively, a new set of traditional, shorter (e.g. 18-24 bp) forward and reverse primers can be utilized for amplification. Restriction endonuclease sites can be engineered into the forward and reverse primers to facilitate downstream cloning.

A synthetic version of K99 native FanC was created by the above sequential pair-wise annealing and extension of complimentary synthetic oligonucleotides methods. Initially, four 20 ul reactions containing 10 pmol of complementary oligonucleotide pairs were assembled on ice. Reaction A contained fanC-plus fanC-8; reaction B contained fanC-2 plus fanC-7, reaction C contained fanC-3 plus fanC-6, and reaction D contained fanC-4 plus fanC-5. In addition, each reaction contained 50 mM NaCl, 10 mM Tris-HCl (pH 7.9), 10 mM MgCl2, 1 mM dithiothreitol (DTT), 1 mM dNTPs, and 0.1 mg/ml bovine serum albumin (BSA). Reactions were heated to 94° C. for 5 minutes and then annealed at 60° C. for 5 minutes. Three units of T4 DNA Polymerase was added to each reaction, and extensions were carried out for 15 minutes at 14° C. The above heating-annealing-extension cycle was repeated a second time with addition of 3 units fresh T4 DNA Polymerase at the extension step. Reaction A was combined with B and reaction C was combined with D to make reactions E and F, respectively. Heating, annealing and extensions were carried out for two cycles as described above with fresh enzyme added prior to the extension. Reaction E was finally combined with reaction F to make reaction G, and subjected to the cycle regimen described above. PCR amplification of template G was carried out using 5 ul of template, 50 pmols each of fanC-9 and fanC-10, 0.2 mM dNTPs, and 5 U of Pfu DNA Polymerase (Stratagene, La Jolla, Calif.) in buffer recommended by the manufacturer. PCR reactions were denatured for 3 minutes at 94° C., and then cycled 25 times (94° C. denaturation for 45 seconds, 60° C. annealing for 30 seconds, 72° C. extension for 1 minute) in a Stratagene Robocycler. Five units of Taq DNA polymerase (Promega, Madison, Wis.) was added to the reaction and incubated continued at 72° C. for 10 minutes to allow 3′ terminal addition of A residues to PCR-amplified products. The sequence of synthetic FanC, designed using the above codon optimization table and constructed as described above, as well as four variants of the fanC sequence are shown in FIGS. 20A-E:

FIG. 20 A is Synthetic FanC nucleotide sequence (designed using the codon table shown in the patent and a back-translation program).

The sequences shown in FIGS. 20B-E are represented by variants of mSEB 1-4, respectively as enumerated in table 5. See FIGS. 20B-E and Table 5 for the mSEB sequences with and with native and glycinin signal peptide regions and with and without various C-terminal extensions. FIGS. 20B-E correspond to variants 1-4, respectively (SEQ ID NOS: 48, 49, 50, and 51, respectively). In FIGS. 20B and C, the native signal sequence is bold and underlined.

In FIG. 20C, the c-terminal KDEL sequence is bold and italicized. In FIGS. 20D and E, the soybean glycinin sequence is bolded and underlined. In FIG. 20E, the 6× histidine sequence is italicized and bolded.

TABLE 5

| Variant | Signal peptide | Gene | C-terminal extension |
|---|---|---|---|
| 1 | Native SEB | Mutant SEB | None |
| 2 | Native SEB | Mutant SEB | ER retention (KDEL) |
| 3 | Soybean glycinin | Mutant SEB | None |
| 4 | Soybean glycinin | Mutant SEB | 6xHis (GGHHHHHH) |

### Using a Service Provider to Design and Construct Synthetic Genes

Over the last decade it has become more common to pay a service provider to provide turn-key synthetic genes for expression in various host systems. One example of the many companies offering such synthetic gene services is GeneArt which is affiliated with Life Technologies (Grand Island, N.Y.). Using the customer portal on the GeneArt website, a desired protein sequence can be entered. Using specialized software (algorithms) developed by GeneArt a specific protein sequence(s) can be back translated using standard or customized codon tables, and synthetic gene sequences can be obtained. The GeneOptimizer software offered by GeneArt, as most other software programs used in the industry, maximizes the expression of synthetic genes for a particular designated expression system (e.g., bacteria, yeast, plants, mammals, etc.). In most cases algorithms are proprietary though many portals allow the entry of protein sequences with an output of what the computer predicts will be an optimal gene for expression. While most algorithms are proprietary, they all share many basic components in common. For example, gene synthesis software programs will be able to remove direct sequence repeats and motifs, adjust codon usage for the host expression system, optimize the GC content of the codons, eliminate motifs that negatively impact expression (e.g. splice sites, polyadenylation sequences, known cryptic sequences that could function as splice sites and polyadenylation sites, etc.), avoid RNA interference sequences, avoid RNA secondary structures that could result in destabilization, eliminate restriction endonuclease sites that could interfere with downstream cloning or are just not desired, and introduce specific restriction endonuclease sites that would facilitate downstream cloning or aid in downstream analyses (e.g. introducing a site to be used for Southern blotting experiments to determine copy number or gene complexity). The GeneArt software will detect potential problems with output sequences if problems exist, and will direct customers to contact company employees that specialize in gene design and can further review the computer predicted optimized sequences. Many companies, including GeneArt, will offer a service to synthesize desired gene sequence(s).

The GeneArt algorithms can be used to design a synthetic gene encoding human thyroglobulin (hTG) for expression in soybean. This synthetic gene is 8.3 kilobases in length and encodes a homodimeric protein with a molecular weight of 660 kilodaltons (kDa). To date there is no recombinant form of hTG protein so current hTG protein (used in basic science research and also used as a major component diagnostic thyroid assay kits) continues to be purified from cadaver thyroids. hTG has been successfully expressed by the inventors in soybean seeds. Soy-derived hTG accumulated to levels representing >1% of total soluble soybean seed protein. The protein also appears to be glycosylated based on its molecular mobility in acrylamide gels. The unique characteristics of soybean seeds, which includes the natural synthesis and storage of large complex proteins (e.g. complexed glycinins and conglycinins), was instrumental in the unprecedented synthesis and accumulation of this large and complex recombinant protein in soybean seeds. Recombinant soy-derived hTG appears to be superior to cadaver-derived and purified hTG as it is more homogenous than commercially-available purified hTG. Thus, recombinant soy-derived hTG can function as a “universal standard” since all recombinant batches will be uniform (unlike different batches of thyroid-purified hTG which are more heterogenous). Furthermore, soy-derived hTG can be produced for pennies per milligram and has applications for novel and affordable medical devices (e.g. uses in thyroid cancer screening) that would otherwise be cost-prohibitive to manufacture and sell commercially. To date, the expression of soy hTG remains the largest recombinant protein to be expressed in any plant system. The inventors explain the process for cloning soy with the hTG in US Patent Application No. 20130243821, which is herein incorporated by reference in its entirety.

Other Companies offer custom gene design and synthesis services but do not allow the user to generate synthetic sequences. Instead, control of the proprietary algorithms remain in the control of the service provider. One example of a gene synthesis company that employs this business model is DNA2.0 based in Menlo Park, Calif. DNA2.0 provides turnkey design and synthesis services and offers one of the fastest turn-around synthesis times in the industry. They, too, use proprietary algorithms based on in-house data generated over the years, which they claim results in optimal gene expression. One advantage of using a gene design and synthesis service provider is the ability to generate variations of a synthetic sequence at a fraction of the cost of the original, or master synthetic sequence.

There are many reasons why a genetic engineer would want to make gene variants. One reason might be to create similar gene sequences with specific point mutations at known locations. Another reason for making a gene variant would be to engineer a gene sequence encoding the amino acids “KDEL” (a universal endoplasmic reticulum retention sequence) at the C-terminus of a protein to test for targeting and accumulation within the E.R. organelle. Another reason for making a gene variant would be to engineer a sequence encoding one of the many known chloroplast targeting signals at the N-terminus of a synthetic gene to test for targeting and accumulation within chloroplasts. Another reason for making a gene variant would be to include a sequence that encodes a signal peptide which would allow proteins to be directed to a secretory pathway for translation and post-translational modifications. Yet another reason for making a gene variant would be to test different signal peptide sequences to determine what role they may play in targeting and accumulation to subcellular locations. Still, another reason for making a gene variant would be to create a “tag” that could facilitate protein purification. One such example of a sequence tag would be a sequence encoding six tandem histidine codons.

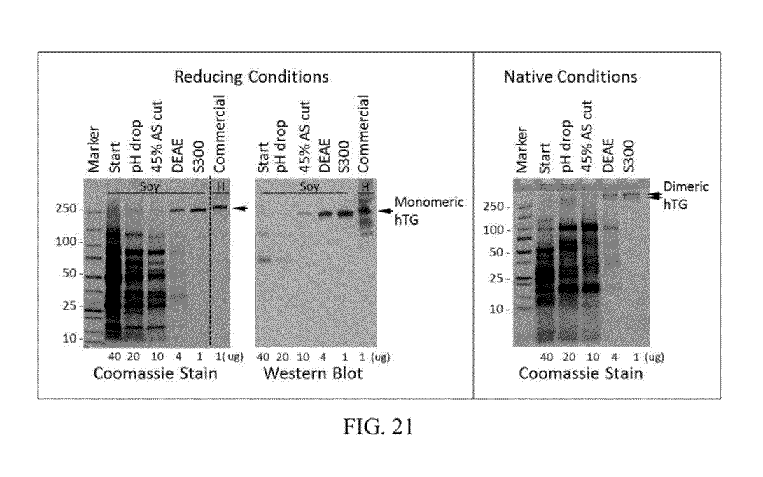

There are commercial companies (e.g., DNA2.0) that can aid in designing and synthesizing a synthetic gene encoding a mutant, nontoxic Staphylococcal enterotoxin B (mSEB) protein. Expression of mSEB in soybean was used to show the feasibility and practicality of soybean as a host for the cost-effective expression of a potential subunit vaccine antigen that could be stored in seeds or as a ground powder for long periods of time under ambient conditions. Since this protein is a potential subunit vaccine candidate, it was engineered with and without a sequence encoding a C-terminal histidine tag for protein expression. The synthetic gene sequences encoding variants of mSEB are shown in FIG. 21.

Targeting Gene Expression and Protein Accumulation to Optimal Subcellular Locations within Soybean

Cellular proteins exist in virtually every compartment, organelle and subcellular location that comprises a cell. Heterologous proteins can be targeted for expression and accumulation in many of these compartments, organelles and subcellular locations, including for example the cytoplasm, endoplasmic reticulum (E.R.), mitochondria, chloroplast, vacuoles, protein bodies, cell membrane, cell wall and apoplastic spaces just to mention some. Protein targeting can be accomplished using a variety of regulatory sequences, including for example chloroplast targeting sequences, prokaryotic and eukaryotic signal sequences, endoplasmic reticulum retention signals and vacuolar sorting signals, etc. to name a few. These regulatory sequences are often relatively short (<30 amino acids, or <90 nucleotides) and are generally found at either the front end or back end of synthetic sequences. For example, some genes encode a front end sequence (e.g. 5′ DNA sequence that encodes an N-terminal amino acid sequence) that functions as a signal peptide and targets protein translation to ribosomes associated with the E.R. (e.g. a secretory pathway for protein translation and post-translational modification). Proteins that reside (e.g. be retained) in the E.R. generally contain a 3′ sequence that encodes the amino acids “KDEL”, which is a common E.R. retention sequence. The presence of a sequence encoding an N-terminal chloroplast transit peptide (CTP) results in targeting to the chloroplast. The absence of a signal peptide usually results in cytoplasmic localization. Thus, the inclusion of specific regulatory or targeting sequences (signals) can be utilized to target the ultimate localizations of heterologous proteins in various expression systems. Subcellular localization is important for many reasons. For example, the protein responsible for allowing RoundUp Ready plants to survive glyphosate spray is localized in the chloroplast, and this is necessary and important because leaves are the specific target for the herbicide spray. Different subcellular locations also provide different biochemical environments that can impact protein stability.

Current knowledge in the field of protein targeting in plants is limited. For this reason, it is often necessary to determine empirically the optimal subcellular location for stable protein accumulation. This can be accomplished using multiple gene variants with different regulatory or targeting sequences. Often there are reports of aberrant or unexpected targeting using a specific sequence, or reports that appear to contradict what is believed to be common practice to those skilled in the art of gene design and protein targeting. One such example was a 2013 report in the journal Science (Goodman et al 2013, Science 342:6157, pp. 475-479.) where Goodman and colleagues characterized >14,000 synthetic reporters in E. coli and found that the use of rare codons in N-terminal sequences resulted in up to 14-fold greater expression. The use of rare codons in N-terminal sequences goes against common knowledge in the filed which has always been to avoid rare codons in the N-terminal sequences when designing genes.

The inventors have discovered that many heterologous proteins targeted to soybean seeds are stable when they contain a sequence encoding a signal peptide. While this is certainly not a universal trend for all plants, it is an observation seen in soybean while expressing several different heterologous proteins over the years. Thus, a generic disclosure of soy without details specific to soy generally does not provide sufficient information (in contrast to the present invention) for one of skill in the art to make transgenic without undue experimentation. For example, a synthetic gene encoding human thyroglobulin (hTG) protein expressed well and accumulated hTG to levels >1% of total soluble protein (TSP) in soybean seeds when the endogenous signal peptide sequence was included in the gene design. Heterologous hTG protein was localized internally and associated predominantly with the cell membrane. Recently the inventors have learned that expression of a heterologous gene encoding a mutant form of the Staphylococcal enterotoxin subunit B (mSEB) protein accumulated to levels >1% TSP when a signal peptide was included in the gene design. In that study, two different signal peptide sequences were evaluated. The first signal peptide sequence evaluated encoded the native SEB bacterial signal peptide while a second signal peptide sequence encoded a soybean glycinin (11S) signal peptide. Both signal peptides present on the soy-derived heterologous proteins were recognized by the plant protein processing machinery as both proteins contained identical mature termini as determined by N-terminal protein sequencing. While the N-termini of both mature mSEB proteins were identical, the subcellular localizations of the two proteins were quite different. Protein encoded with the native SEB signal peptide was localized to apoplastic spaces while protein encoded with the soybean 11S signal peptide was localized intracellularly and associated predominantly with the cellular membrane. These results indicated that (1) inclusion of a signal peptide (regardless of origin) resulted in stable protein accumulation, and (2) the native SEB leader peptide may possess signals to direct heterologous proteins to apoplastic spaces. These results are contrary to results published by Chickwamba and colleagues (Proc Natl Acad Sci USA. 2003 Sep. 16; 100(19):11127-32), which is hereby incorporated by reference in its entirety. In that study, heterologous expression of E. coli labile toxin subunit B (LT-B) in maize resulted in LT-B protein accumulation in maize starch granules regardless of whether the native LT-B signal peptide or a plant chitinase signal peptide was included in gene design. The report by Chikwamba et al prompted Elizabeth Hood to write a commentary entitled “Where, oh where has my protein gone?” (Trends Biotechnol. 2004 February; 22(2):53-5.). In that article, Hood writes “A recent publication by R. Chikwamba and colleagues highlights interesting issues in recombinant protein expression in transgenic plants. In the study, they expressed a bacterial antigen in maize seed and obtained aberrant localization data. This work is of great importance to the biotechnology industry and raises fascinating questions in plant cell biology that require creative thinking”.

While the studies of Chikwamba supports the presence of targeting sequences within a protein, the inventors' studies suggest a potential role for the SEB leader peptide in targeting proteins to the apoplast. It has been found that the heterologous mSEB protein accumulates to levels >1% of TSP when localized within the apoplast when the bacterial signal peptide was utilized. It is possible that the native SEB leader peptide functions as an apoplast targeting signal and that the apoplast represents a favorable subcellular location in soybean seeds that supports stable accumulation of recombinant protein. Until more is learned about protein targeting in soybeans, the inventors have discovered that it is prudent to include several synthetic gene variations containing different targeting sequences to evaluate heterologous protein accumulation and stability (as is shown for example, in FIGS. 20A-E). Given past experiences, the inventors know that synthetic gene design for heterologous expression of protein in soybean seeds should include a signal peptide to allow target proteins to accumulate to optimal levels. In soybean expressions, the inventors have consistently observed >1% TSP expression in soybean seeds with inclusion of a signal peptide sequence in the gene design. Furthermore, the inventors know that intracellular membrane and apoplastic spaces are preferred subcellular locations for optimal heterologous protein accumulation and stability.

An example of the types of synthetic gene variants that can be synthesized and tested for evaluation in soybeans is shown below in Table 6. In that example, the target protein is a human myelin basic protein (hMBP) fusion. Three types of signal sequences can be evaluated by making gene variants encoding different signal peptide sequences. One signal peptide sequence encodes the hMBP native signal peptide, a second sequence encodes the SEB signal peptide, and a third sequence encodes the chitinase signal peptide derived from the model plant Arabidopsis thaliana. A forth gene variant would not encode any signal peptide sequence while a fifth variant would encode a plant signal peptide along with a KDEL universal E.R. retention signal. These variants should direct heterologous hMBP to a preferable subcellular location within the soybean seed and identify the specific location(s) that result in the most optimal location with respect to stable protein accumulation.

TABLE 6

| Type of sequence | Source of sequence | Encoded amino acid sequence |
|---|---|---|
| Signal peptide | Native hMBP | MEPWPLLLLFSLCSAGLVLG |
| | protein | (N-terminal) SEQ ID NO: 52 |
| Signal peptide | S. aureus SEB | MDKRLFISHV ILIFALIL STPNVLA |
| | protein | (N-terminal) SEQ ID NO: 53 |
| Signal peptide | A. thaliana | MPPQKENHRTLNKMKTNLFLFLIFSLLLSLSSA |
| | chitinase protein | (N-terminal) SEQ ID NO: 54 |
| No signal peptide | N/A | N/A |
| Signal peptide + | A. thaliana | MPPQKENHRTLNKMKTNLFLFLIFSLLLSLSSA |
| E.R. retention | chitinase | (N-terminal) + KDEL (C-terminal) |
| | protein + E.R. | SEQ ID NO: 55 |
| | retention consensus | |

Other gene variants can be synthesized to encode human acetylcholine receptor (hACR) fusion proteins. Still other gene variants can be synthesized to encode any of the known enzymes involved with the deconstruction of lignin, hemicellulose, or cellulose, or any yet unknown enzyme involved with the deconstruction of lignin, hemicellulose, or cellulose (for example, as shown in Tables 1, 2, and 3).

### Selection of a Promoter and Regulatory Elements to Drive Optimal Expression of Heterologous Proteins in Soybean Seeds

A first step for efficient production of heterologous protein in any recombinant system is to maximize the levels of foreign protein expression. Since transcription is one of the earliest steps in the process of protein production, it is a place to focus when attempting to increase recombinant protein yield. Soybean tissue-specific promoters express in a spatial and temporal manner and allow heterologous proteins to be expressed in specific locations at specific times of seed development. Some examples of soybean seed-specific that are ideal for heterologous expression of protein in soybeans include the β-conglycinin (7S) and glycinin (11S) promoters. The use of these or similar promoters should result in heterologous protein accumulations of >1% of TSP.

Some sequences are known to function as enhancer sequences, either for transcription or translation. One sequence identified in Tobacco Etch Virus has been shown to function as a translational enhancer. A preferred synthetic gene design will contain enhancer sequences such as that derived from TEV, or other sequences that increase or enhance transcription or translation. These sequences can be derived from specific gene leader sequences (e.g. 5′ untranslated regions), introns, or other sequences shown to enhance transcription or translation.

Some sequences are known to function as terminator sequences and contain polyadenylation signals and other sequences important for mRNA transcript recognition. Typical sequences that function as effective terminator elements in soybean include the Nos terminator, the 35S terminator, the Bar terminator, and the vegetative storage protein terminator, just to mention some. The terminator regulatory sequence is typically placed downstream of the gene of interest open reading frame (ORF).

A typical synthetic gene for expression in soybean seeds will thus contain a (1) a seed-specific promoter (e.g. 7S, or 11S) followed by an enhancer element sequence (e.g. the TEV translational enhancer) followed by a sequence encoding a signal peptide (e.g. the native SEB signal peptide, the Arabidopsis thaliana plant signal peptide or the gene of interest signal peptide) followed by the open reading frame of the desired protein product (e.g. one or more enzymes involved with the deconstruction of lignin, hemicellulose or cellulose) followed by a terminator sequence (e.g. 35S, Nos, Bar, vsp, etc.). Together, these above elements constitute a gene cassette.

For example, a soybean codon-optimized gene containing sequences encoding a signal peptide is synthesized by GeneArt, DNA2.0 or a similar service provider. Restriction endonuclease NcoI and XbaI are engineered on the 5′ and 3′ termini to facilitate subcloning. Following digestion with NcoI and XbaI the synthetic gene are isolated from an agarose gel and ligated into linearized pPTN200 vector. The resulting vector construct will therefore contain the 7S β-conglycinin promoter, Tobacco Etch Virus (TEV) translational enhancer, desired signal peptide, desired open reading frame, and 35S terminator. The pPTN200 vector backbone was previously engineered to contain a cassette encoding phosphinothricin acetyltransferase (bar gene) under the control on the nopaline synthase promoter and terminator elements. Following subcloning, verification of the gene cassettes can be carried out using standard DNA sequencing methods.

### Soybean Transformation

Soybean (Glycine max) can be readily transformed by an array of different transformation methods which have been developed and optimized over the past decade in various laboratories. Two of the most successful and widely used transformation techniques are the cotyledonary node transformation using the bacteria Agrobacterium tumefaciens, and the particle bombardment of somatic embryogenic cultures. Regeneration using somatic embryogenesis has been reported using a variety of explant tissue including embryonic axes, intact zygotic embryos, and excised cotyledons. Other, less commonly used, methods have been developed to transform soybean. One example is the introduction of exogenous DNA into a plant embryo through the pollen tube pathway after pollination. Another example is the use of Agrobacterium rhizogenes. This bacterium causes hairy root disease and is used in a manner similar to A. tumefaciens to infect wound sites on roots and transfer T-DNA from the bacterial cell to the plant cell. Other methods of soybean transformation that have been mentioned in the literature include electroportation, silicon carbide fibers, liposome-mediated transformation and in planta Agrobacterium-mediated transformation using vacuum infiltration of whole plants.

There are several ways to perform soybean transformation. In one example, a binary vector harboring a gene of interest cassette and a plant selectable marker cassette is mobilized into Agrobacterium tumifaciens strain EHA101 by triparental mating. Soybean (Glycine max Merr) genotype Thorne (Ohio State University) or a similar strain that is transformable with Agrobacterium is used for transformation with the above resultant trans-conjugant. Glufosinate is used as the selective agent (assuming the plant selectable marker is the Bar gene) at concentrations of 5 mg/ml and 3 mg/ml during shoot initiation and elongation steps, respectively. Following regeneration, young plantlets are transplanted to soil and maintained in a greenhouse. After about 4 weeks in soil, leaf trifoliates can be isolated and used for molecular characterizations (e.g. Southern blots to determine T-DNA complexity).

In a method, soybean transformation is carried out using a Agrobacterium-mediated half seed method. Using this method, half-seed explants (Glycine max) are dissected and inoculated with Agrobacterium suspension culture (strain EHA101 carrying various binary vectors). The inoculated explants are placed adaxial side down on co-cultivation medium at 24° C. and under 18:6 photo period for 3-5 days. After co-cultivation, explants are cultured for shoot induction and elongation under glufosinate selection (8 mg/L) for 8-12 weeks. Herbicide resistant shoots are harvested, elongated and rooted as described. Acclimated plantlets are transferred to soil and grown to maturity in the greenhouse.

### Molecular Characterization of Transgenic Plants and Transgenic Seeds

There are several different methods that can be used to characterize transgenic soybean plants and transgenic seeds. These methods and assays can confirm the presence of heterologous transgenes and protein, determine complexity of the transformation event, quantify levels of heterologous protein, and allow for identification of subcellular localization. Some typical assays include (1) foliar sprays with herbicide to screen for the expression of the plant selectable marker, (2) isolation of genomic DNA for use in PCR to screen for the presence of a desired transgene, (3) isolation of genomic DNA for use in Southern blot analyses to determine T-DNA insert complexity), (4) isolation of RNA to screen for the presence of transgenic mRNA, (5) isolation of soluble protein for use in western blot analyses to screen for the presence of transgenic protein and determine observed molecular mobility, (6) isolation of protein for use in ELISAs to screen for transgenic protein or quantify levels of transgenic protein in soluble extracts, and (7) confocal microscopy to identify subcellular localization of heterologous protein. The above assays and the associated methods are common and well-known to those skilled in the art. These methods and assays are incorporated into the examples shown in this application.

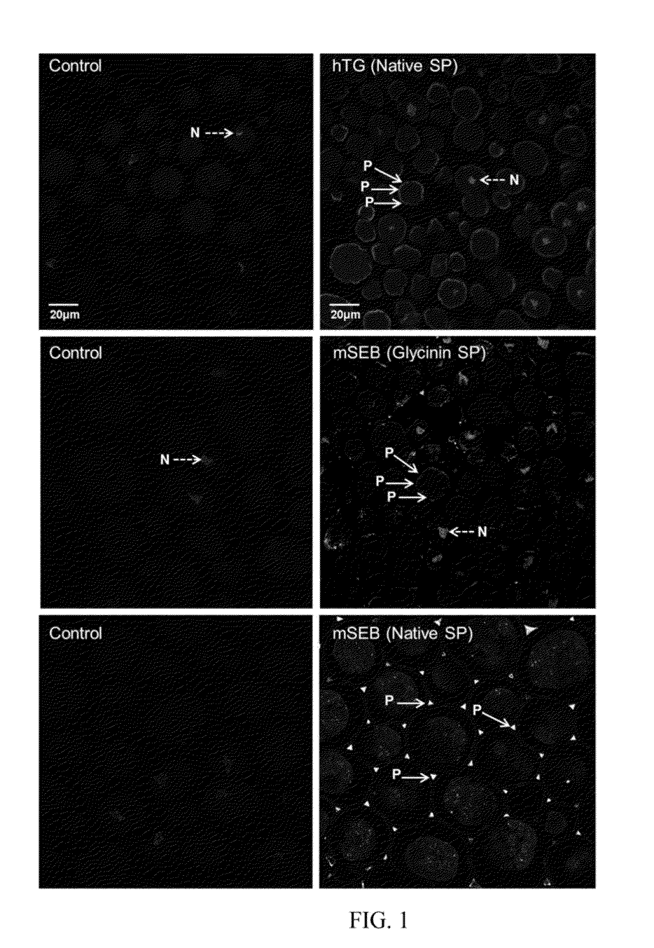

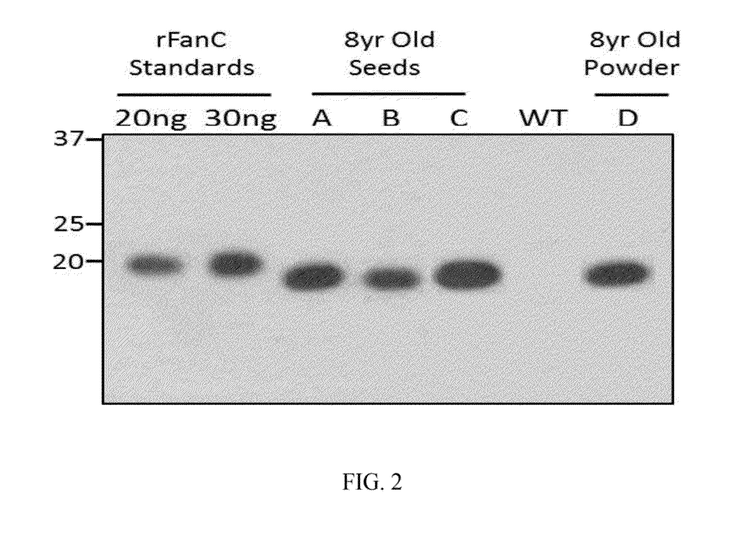

FIG. 1 illustrates immunofluorescence showing subcellular localization of different heterologous proteins in soybean seeds. Control seeds are shown in the left panels and transgenic seeds are shown in right panels. Samples were viewed at 20× magnification using confocal microscopy, and identical microscope parameters were used for photography. Each heterologous protein contained an N-terminal signal peptide. The subcellular location of heterologous protein is indicated by solid arrows with the designation “P” while nuclei stained with DAPI are indicated with a dashed arrow with the designation “N”. Panel A shows immunofluorescence of human thyroglobulin (hTG) protein which contained the native hTG signal peptide and is localized to the intracellular membrane. Panel B shows immunofluorescence of heterologous S. aureus mutant enterotoxin B (mSEB) protein which contained a soybean glycinin signal peptide and is localized to the intracellular membrane. Panel C shows immunofluorescence of heterologous S. aureus mutant enterotoxin B (mSEB) protein which contained the S. aureus native SEB signal peptide and is localized to apoplastic spaces. In each case heterologous protein accumulated to levels >1% of total soluble protein and demonstrates the importance of a signal peptide for stable protein accumulation.

Grinding Soybean Seeds Containing Heterologous Proteins into Powder Compositions

While heterologous transgenic proteins remain stable in soybean seeds for many years (and potentially decades) there are advantages to grinding those seeds into powder compositions. One reason for grinding transgenic seeds into a powder is to render them non-viable for germination. Transgenic soybean seeds expressing heterologous proteins that have not been approved for specific use must be contained at all times to prevent escape into the environment and contamination of global food supplies. However, once transgenic seeds are ground into a powder (or rendered nonviable by any of a variety of methods) they are often not subjected to the same strict containment regulations (assuming the heterologous gene or protein products present in the powder do not represent bio-hazards). In the United States, viable transgenic soybeans cannot be transported across state borders without an APHIS USDA movement permit, however, there are no such regulations for the movement of transgenic ground seed powder. Grinding soybean seeds into powder therefore creates flexibility for this expression system as seeds expressing lignocellulosic deconstruction enzymes or other heterologous proteins can be grown and harvested at one location, and then ground into a powder and shipped to various other locations for use in degradation.

Another reason for grinding transgenic soybeans expressing heterologous proteins into seed powder is to facilitate protein extraction. There is a direct correlation between soybean powder particle size and protein extractability. Simply stated, greater levels of soy protein can be extracted from seeds ground finely into a powder than from either intact seeds or seeds ground into a coarse powder. In addition, soy protein can be extracted much quicker from a finely ground seed powder than from intact seeds or coarsely ground seed powder. These extraction and solubility properties are directly associated with the available surface areas of seeds and various-sized particles that comprise ground seed powders. In the present invention it is desired that heterologous proteins expressed in soy solubilize quickly (and with minimal agitation) when added to aqueous mixtures containing appropriate substrates. In this regard, soybean seeds ground into powders with relatively small particle sizes will best accomplish this goal.

Grinding soybeans into powder can be accomplished at smaller scale levels using standard coffee grinders and blenders with wave action blades (or using available micronizers).

Grinding can also be accomplished at larger scales using industrial equipment scaled appropriately for the desired application. As a general rule, seed powders containing coarse particles are obtained with relatively less grinding while seed powders containing finer, smaller particles can be achieved with relatively more grinding. It is important not to overheat the blades or soy sample during grinding process as seeds contain small amounts of water (usually 9-15% by weight) and release of this water in combination with the heated blades create conditions that support formation of undesired soy slurries.

In some cases it is prudent to remove seed hulls before grinding seeds into a powder. Seed hulls are relatively hard and are more difficult to grind than the seed. The presence of hulls in the grinding process will result in powders containing coarse particles. Furthermore, soybean seed hulls contain little protein (they are fiber-rich) yet they comprise ˜10% of the dry mass of soybeans. Therefore, removal of soybean hulls effectively increases the protein concentration potential of the powder. Yet another reason to remove hulls prior to grinding is that this invention does not target heterologous protein expression to seed hulls. Instead, heterologous proteins are targeted to intracellular seed compartments and the apoplast. Protein extracted from soybean seed hulls is devoid of heterologous protein.

Soybean hulls can be removed from seeds using a variety of cracking methods that can be accomplished by hand or by machine. The simplest methods for cracking seeds involves mechanical disruption of the seed coat, using for example, a heavy object, a rolling pin, or a grinder or blender with short pulses. Following disruption the seed “meats” can be separated from the hulls by hand. This process can be time consuming and may not be practical for larger applications. There are also machines that are capable of separating soybean hulls from meats. These machines use mechanical force to “crack” a seed into ˜16-32 pieces, and then a combination of sieves, screens and air fans work together to remove the soy hulls. Efficiencies of de-hulling machines are typically high (e.g. >98% recovery).

### Importance of Soybean Seed Powder Particle Size

As mentioned above, there is a direct correlation between protein extractability and seed powder particle size. In the present invention, one or more soy powders containing enzymes capable of deconstructing lignin, hemicellulose and cellulose are added to a liquid substrate. Soy protein containing the deconstructing enzymes are then solubilized and begin to carry out their specific enzymatic functions. Particle size is important for rapid and efficient solubilization of soy protein. In general, smaller seed powder particles will elute more protein than an identical weight of larger powder particles if aqueous volume and time are kept constant.

After soybean seeds are ground, the powder can be passed over a series of sieves or screens to separate the powder particles according to size. Standard sieves, screens and filters are available to create various compositions of seed powder containing pre-determined particle sizes. For example, sifting pans can be purchased from Sigma and mesh filters with various cut off sizes (e.g. 20 mesh, 30 mesh and 50 mesh) can be purchased from Bellco Glass Inc. The mesh screens are held in place by a retaining ring located at the bottom of the sifter pan and tightened with a specialized key.

### Transgenic Seeds

To determine solubility of different particle sizes, transgenic seeds expressing various heterologous proteins can be ground in a coffee mill to a powder and then particles with various size diameters can be separated with the sifting pan and mesh filters with different diameter cut offs. For example, 10 mesh filters have a 1910 micron diameter cutoff; 20 mesh filters have a 860 micron cutoff; 30 mesh filters have a 520 micron cutoff; 50 mesh filters have a 280 micron cutoff, 100 mesh filters have a 140 micron cutoff, 300 mesh filters have a 46 micron cutoff; and 500 mesh filters have a 25 micron cutoff. Ground powder can first be sifted through to 10 mesh sieve. Particles that pass through this mesh are <1910 microns in diameter while those trapped by this mesh are >1910 microns in diameter. Particles that passed through the 10 mesh screen are then passed through the 20 mesh screen. Those that pass have diameters <860 microns while those that do not pass through (e.g. trapped between the two meshes) have diameters between 860 microns and 1910 microns. Powder is then passed through the 30 mesh filter. Particles that are trapped by this filter have diameters between 520 microns and 860 microns. This process of sieving and trapping particles is continued until particles are too big to pass through the chosen mesh. At this point the powder will need to be further ground if smaller particles are desired. The table below shows the different compositions that can be obtained using the sieving process described above.

TABLE 7

| | Mesh | | Method for | Particle size in | |
|---|---|---|---|---|---|
| Composition | size | Mesh cutoff | obtaining particies | composition | |
| 1 | 10 | 1910 microns | 10 mesh cutoff | >1910 | microns |
| 2 | 10 | 1910 microns | 10 mesh + 20 mesh trap | 860-1910 | microns |
| 3 | 20 | 860 microns | 20 mesh + 30 mesh trap | 520-860 | microns |
| 4 | 30 | 520 microns | 30 mesh + 50 mesh trap | 280-520 | microns |
| 5 | 50 | 280 microns | 50 mesh + 100 mesh trap | 140-280 | microns |
| 6 | 100 | 140 microns | 100 mesh + 300 mesh trap | 46-140 | microns |
| 7 | 300 | 46 microns | 300 mesh + 500 mesh trap | 25-46 | microns |
| 8 | 500 | 25 microns | 500 mesh pass through | <46 | microns |

It should be noted that this method represents one of many methods that can be used to obtain or separate powder to specified size classes. For example, there are grinders that can be set or programmed to yield a particular particle size class.

Soybean seed powder compositions containing various size class particles can be then tested for solubility by mixing a known mass with a known volume of aqueous solution for a specified time period. For example, 100 mg of powder can be added to 10 ml of an aqueous solution and gently inverted for 1 minute, 30 minutes, 60 minutes, or any other time. The solubilized protein solution is then clarified by centrifugation, filtering, or any method that can separate the soluble material from the insoluble material. The solubilized protein samples can then be quantified and characterized. Examples of the types of information that can be obtained from sample characterizations include but are not limited to (1) visualization of solubilized protein compositions in native and denaturing acrylamide gels following staining with Coomassie blue dye, (2) visualization of specific heterologous proteins following western blot analyses, (3) determination of solubilized total soy protein using protein quantification assays such as the Bradford assay, (4) determination of the absolute amounts of soy protein and/or heterologous protein solubilized over a given time period, (5) Determination of kinetics for soy protein and/or heterologous protein solubilization, and (6) determination of total soy protein and/or heterologous protein solubilized as a percentage of total gross protein.

Solubility of Soy Protein and Heterologous Protein in Compositions with Varying Seed Powder Particle Sizes:

Ideally, the solubilization of soy powder added to an aqueous solution should occur in a rapid manner. Based on surface area and other biophysical properties, seed powder compositions containing particles with smaller average diameter sizes will have a faster dissolution rates relative to compositions containing particles with larger average diameter sizes.

Dissolution rates of bulk soy protein and heterologous protein can be determined for different soybean seed powder compositions. In addition, absolute levels of bulk protein and heterologous protein solubilized from a known starting mass of soy powder in a specified volume of aqueous solution over a given time can also be measured. To illustrate this point, the following example can be used to determine the amount of bulk soy protein that is solubilized in a powder composition comprising ground seed particles with a hypothetical mean diameter of 250 microns. An appropriate experiment would involve the addition of 100 mg of the powder composition to a 10 ml sample of aqueous buffer (e.g. TE or PBS). The aqueous solution containing the powder composition would be gently inverted for 1 minute and then clarified by either centrifugation, filtering or any appropriate method that allows for the separation of aqueous solution from solid particles. A protein quantification (e.g. Bradford assay) could then be used to determine the absolute level of soy protein that was solubilized under the above specific conditions. If that number revealed that 20 mg of protein was recovered, it can be concluded that ˜50% of the available soy protein was solubilized in this particular composition (100 mg starting sample contains 40 mg of available protein of which 20 mg or 50% is solubilized). Other methods of quantification are contemplated and therefore within the scope of the present invention; This type of information can be obtained for other compositions defined by particle size and collectively used to create optimal compositions for various different applications.

Further calculations can be performed to determine whether a specific heterologous protein is solubilized at the same rate as bulk soy protein, or alternatively, is solubilized faster or slower than bulk soy protein. Experiments involving western blots and ELISAs would help in such determinations. If it is known that a specific heterologous protein represents 1% of total soluble soy protein, and then determined that protein samples solubilized for 1 minute also contained heterologous protein representing 1% of total solubilized protein, it can be concluded that heterologous protein and bulk soy protein have similar solubilization rates. Alternatively, if the amount of target protein in the solubilized sample was >1% of TSP then the heterologous protein became soluble at a rate greater than that for bulk soy protein; similarly if the target protein represented <1% of TSP the heterologous protein solubilized at a rate slower than that of bulk soy protein. It is believed that proteins with lower molecular masses will solubilize faster than proteins with higher molecular masses (assuming particle size, starting mass and aqueous volume remain constant).

The information collected from the types of experiments and calculations outlined above could be used to create custom powder compositions for various different applications. For example, assume that protein from 50 micron particles containing enzyme A is solubilized in 1 minute while protein from 750 micron particles containing enzyme B requires 10 minutes for desired solubilization. A composition could be made by mixing 50 micron particles (containing enzyme A) and 750 micron particles (containing enzyme B) and this unique composition could be practical for applications that required sequential release or timed release of the two enzymes. If it was determined that the reaction was not driven to completion, and more enzyme B was needed, a powder composition containing only enzyme B could be added to drive the reaction to completion. This hypothetical example was chosen to demonstrate the flexibility of the present invention. Accordingly, it should be understood that there is a temporal aspect to the present invention that allows one to ideally catalyze certain reactions at different times. For example, with one solution containing two or more of the enzymes that are enumerated in Tables 1, 2, and 3, one might first metabolize and/or deconstruct lignin and then sequentially metabolize and/or deconstruct cellulose.

### Long Term Storage of Seeds and Ground Powder

Soybean seeds are susceptible to spoilage and reduced germination, especially if moisture levels are not controlled. To reduce these susceptibilities, it is recommended that soybeans be dried (either naturally or with fans) to a residual moisture content of about 9 and/or about 13%, or alternatively, between about 9% and 13%. The moisture content can be determined easily using portable moisture meters. As a general rule, the dryer the seed the longer it will store. For ideal storage, storage temperatures should remain 35-40° F. in winter and 40-60° F. in summer although it should be understood that soybean seeds may be stable at other temperatures (such as warmer temperatures). As a general rule, the cooler the storage temperature the longer it will store. Growers storing seed should provide aeration to any bins that store soybeans.

Given the susceptibility to spoilage and reduced germination rates, along with the processes and cost involved with drying of soybeans, most soybean growers choose not to store seed and instead purchase fresh seed each year prior to planting. Some soybean growers do save seed from one harvest until the next season for planting, but this is a diminishing practice. Relatively few soybean growers practice long term storage of soybeans (e.g. >1 year) for the reasons stated above.

Spoilage is an issue with any oil crop since oils can become rancid. While soybean is the richest natural source of protein known, it is generally recognized as a major oil crop (soy contains ˜20% oil by dry weight). If the oil goes bad in soybeans then the seeds are of little value in the commodities market. Likewise, if soybeans do not germinate they are of little value to soybean growers. While spoilage and decreased germination are major concerns for soybean growers, these issues are of much less concern in the present invention. The present invention is not dependent on a soy-based product that will be consumed by humans, so spoilage of oils is not an issue (as long as spoilage of oils does not impact the stability of heterologous proteins present in the seed). Similarly, this invention is not dependent on seeds being able to germinate following long term storage. While seed banks will certainly need to be maintained, the bulk of harvested seed could be stored for many years regardless of its ability to germinate. Thus, the present invention is dependent upon heterologous proteins in soybean seeds remaining stable for extended periods of time.

### Example

### Use of Various N-Terminal Signal Peptides to Target Heterologous Proteins to Favorable Subcellular Locations of Soybean Seeds for Optimal Accumulation and Stability

Signal peptides are typically short 15-30 amino acid sequences present at the N-terminus of proteins targeted for translation via the secretory pathway. Signal peptides do not show sequence similarity but instead share a common tripartite structure comprising a short N-terminal hydrophobic region with positively charged amino acids, a long core of hydrophobic amino acids that can form an alpha-helix, and a neutral but polar C-terminal region containing the signal peptide cleavage site. Proteins containing signal peptides are generally destined for specific intracellular locations (e.g. the ER, golgi and endosomes) or are secreted. The inventors have found that sequences encoding bacterial signal peptides, plant signal peptides, or other eukaryotic signal peptides (e.g. the native signal peptide present on human thyroglobulin protein) result in stable accumulation of heterologous protein in soybean seeds. Confocal localization has revealed two preferred subcellular locations for stable accumulation of heterologous protein in soybean seeds. One location is the intracellular membrane and a second is the apoplast.

The subcellular location of a heterologous protein can be determined by performing confocal microscopy with appropriate antibodies. FIG. 1 shows examples of confocal images following immunohistochemistry that allowed visualization of the subcellular location of heterologous hTg, and mSEB proteins expressed in soybean seeds. These proteins contained different signal peptide sequences. The hTg synthetic gene encoded a 19 amino acid native signal peptide; the mSEB synthetic gene variants were engineered to contain either the 29 amino acid native bacterial SEB signal peptide or the 22 amino acid soybean glycinin signal peptide.

Whole seed tissues expressing hTg and mSEB were imbibed for 16 hours in 1×PBS and seed coats were removed. Tissues were then fixed essentially as described previously by our laboratory (Piller 2005, Oakes 2008, Powell 2011). Sections were permeabilized with 1×PBS containing 0.2% Tween for 10 minutes, and nonspecific binding was blocked by incubation with 1× PBS supplemented with 3% BSA for 4 hours at 23° C. Tissues were then incubated with either rabbit anti-hTG serum or rabbit anti-mSEB (1:20 dilution) for 16 hours at 4° C., followed by incubation with an AlexaFlour594 goat anti-rabbit IgG-HRP conjugated secondary antibody (1:200 dilution) for 1 hour at 23° C. Finally, tissues were incubated with 4,6-diamidino-2-phenylindole (DAPI; 1:500 dilution) for 5 minutes. Cover slips were added to the sections using Gel/Mount aqueous mounting media. Images were collected with a LSM 710 Spectral Confocor 3 Confocal Microscope (Carl Zeiss, Inc.) using a 40× objective and a 405 nm laser to visualize DAPI stained nuclei, along with a 561 nm laser to collect emitted fluorescence from the alexafluor. Stacks of images (30 optical sections, 17 nm apart) were collected in the Z plane of the specimens and projected to form a single image. To improve clarity and reproduction quality, image colors were proportionally enhanced using the ZEN 2009 Light Edition software. FIG. 1 shows that heterologous hTG protein, engineered with the native hTg signal peptide, localized intracellularly and was strongly associated with the cellular membrane (top panel). Similarly, mSEB engineered with the glycinin signal peptide was also localized intracellularly and was associated with the cellular membrane (middle panel). However, the mSEB protein, engineered with the native bacterial SEB signal peptide, was secreted from cells and accumulated in apoplastic spaces (bottom panel). In the case of mSEB, an identical protein was targeted to two separate locations when different signal peptides were utilized. This result suggests the SEB signal peptide may also contain internal sequences or signals that can direct proteins to apoplastic spaces. The apoplastic space is a favorable environment for accumulation of mSEB, as the recombinant protein accumulated to levels >1% of total soluble seed protein (TSP). It is possible that the SEB signal peptide may target other proteins, such as those enumerated in Tables 1, 2 and 3, to apoplastic spaces. If this site represents a preferred biochemical environment for heterologous protein accumulation, then the SEB signal peptide would represent a valuable tool for accomplishing such targeting.

Association with the intracellular membrane also appears to represent a favorable subcellular location for heterologous protein accumulation in soybean seeds. The synthetic genes for hTg and mSEB both contained sequences encoding a eukaryotic signal peptide, and both heterologous proteins also accumulated to levels representing >1% of TSP.

It is believed that the inclusion of a signal peptide will result in optimal expression and accumulation of the proteins enumerated in tables 1, 2 and 3. Many of those proteins contain native signal peptides that can be included in synthetic gene design. However, some of the proteins in Tables 1, 2 and 3 do not contain signal peptides (e.g. Caldicellulosiruptor bescii 1,4-beta gluconase from Table 7A). Computer algorithms have been developed to recognize the characteristics of signal peptides (e.g. the tripartite structure) and predict the site of cleavage within the signal peptide, and many are easily accessible on the internet. One such signal peptide prediction program is SignalP 4.1 hosted by the Center for Biological Sequence Analysis and can be found at: http://www.cbs.dtu.dk/services/SignalP/. Another example of a server-based prediction program is Phobious (http://www.cbs.dtu.dkiservices/SignalP/) while a third is Signal-Blast located at http://www.cbs.dtu.dk/services/SignalP/.

The SignalP server was used to identify signal sequences and predict where cleavage would occur during synthetic gene design of the hTg and mSEB sequences. Signal peptide prediction programs are especially useful when splicing a signal sequence derived from one protein onto the gene sequence of a different protein. This was the case with the mSEB in which the soybean glycinin signal peptide sequence was spiced onto the bacterial mSEB sequence. Splicing a signal sequence from one protein onto another has the potential to introduce changes in charge and structure that may alter the specificity of the original signal peptide cleavage site. Unanticipated, or non-specific cleavage can result in proteins with alternate N-termini, and there is no way to determine whether such proteins will be as stable as their native counterparts. Thus, it is important to design genes with functional signal sequences that are predicted to yield heterologous proteins with N-termini that are identical to those observed in nature. In cases where predicted signal cleavage sites are different than desired sites, one or a few amino acids surrounding the cleavage site can usually be modified (e.g. changed) and the sequences re-run through the prediction programs until a sequence with a desired cleavage site is obtained.

Signal peptide prediction software was used to identify signal peptides on the sequences enumerated in Tables 1, 2, and 3. By way of example, the signal peptides from randomly selected proteins chosen from Tables 1, 2 and 3 are shown below. The amino acid sequences of these proteins were entered into the Phobius and Signal-Blast prediction programs which both identified identical predicted signal peptide sequences. Table 7A below also shows the native hTg, native mSEB, and soybean glycinin signal peptides previously utilized in the inventors' laboratory. The signal peptide sequence from Arabidopsis thaliana chitinase protein is also included since it has been used by the inventors and others to successfully target heterologous proteins to the secretory pathway.

TABLE 7A

| | Amino | Signal |
|---|---|---|
| Protein | Acids | Peptide Sequence |
| Human thyroglobulin | 1-19 | MALVLEIFTLLASICW |
| | | VSA |
| | | SEQ ID NO: 56 |
| Staphylococcus aureus | 1-29 | MDKRLFISHV |
| enterotoxin B (SEB) | | ILIFALILVI |
| | | STPNVLA |
| | | SEQ ID NO: 57 |
| Glycine max glycinin | 1-22 | MAKLVFSLCFLLFSGCC |
| | | FAFSM |
| | | SEQ ID NO: 58 |
| Arabidopsis thaliana | 1-33 | MPPQKENHRTLNKMKTN |
| chitinase | | LFLFLIFSLLLSLSSA |
| | | SEQ ID NO: 59 |
| Trametes versicolor | 1-20 | MGLQRFSFFVTLALVAR |
| laccase (Table 1) | | SLA |
| | | SEQ ID NO: 60 |
| Talaromyces | 1-22 | MARFSILSTIYLYILFI |
| emmersonii | | GSCLA |
| Xylanase (Table 2) | | SEQ ID NO: 61 |
| Myceliophthora | 1-17 | MTLQAFALLAAAALVRG |
| thermophile glycoside | | SEQ lD NO: 62 |
| hydrolase family 3 | | |
| (Table 3) | | |
| Caldicellulosiruptor | None | None |
| bescii 1,4-beta | | |
| gluconase (Table 3) | | |

Note that there was no predicted signal peptide for Caldicellulosiruptor bescii 1,4-beta gluconase (see Table 3 and 7A) so a synthetic gene encoding this enzyme would be designed with one of the other signal peptides previously shown to function in soybean (e.g. hTG, native mSEB or soybean glycinin). Signal prediction software would then be utilized to predict the cleave site within the engineered amino acid sequence. If accurate cleavage was not predicted, then one or more amino acids surrounding the cleavage site would be modified until a desired predicted site was obtained.

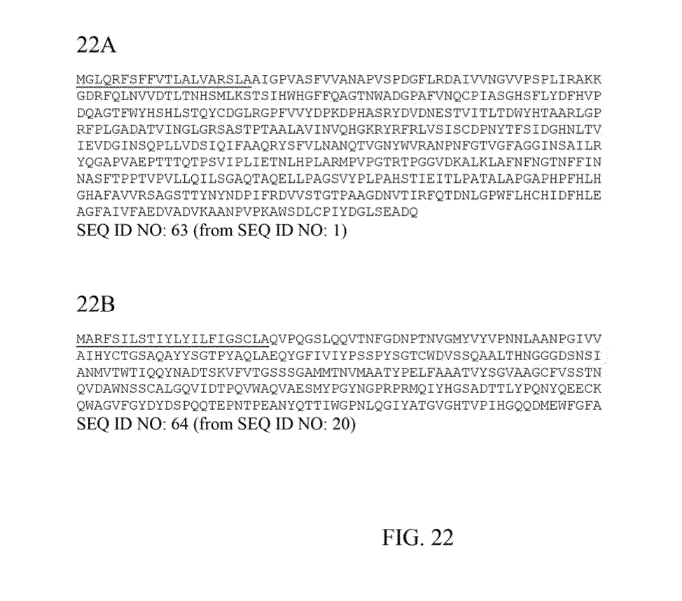

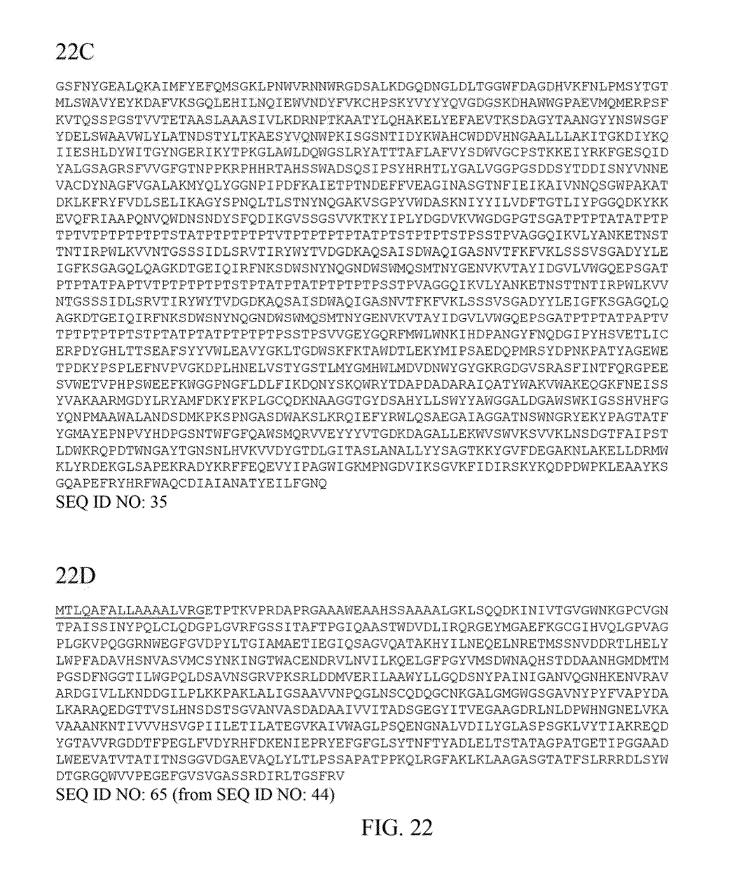

Moreover, as FIGS. 22A-D, please find 4 sequences that were queried using the signal prediction software enumerated above. The full sequences are shown and the signal peptides are underlined. The sequences are signal peptides that have been added (or not) to enzymes from Tables 1, 2 and 3 that have been queried in a signal prediction programs (one from each of Tables 1, 2 and 3 (FIGS. 22A, 22B, and 22D, respectively)) and a second one from table 3 that contains no signal peptide sequence (FIG. 22C). The full sequences are shown and the signal peptides are underlined.

### Example

### Long Term Storage of Intact Seeds and Seed Powder for 8 Years with No Degradation of Heterologous Protein

While a major function of most seeds is to remain dormant until favorable conditions are present for germination and reproduction, this is clearly not the case with soybeans. Soybeans typically maintain viability for ˜1 year if stored properly. Following storage for years 2-5, even under ideal conditions, germination rates drop quickly. While it may be anticipated that heterologous proteins would remain stable in seeds such as maize, wheat, rice, etc. following prolonged storage, it is not obvious that this would also be the case following long term storage in soybeans given that soybeans do not store well. To determine whether heterologous proteins expressed in soybeans can remain stable over long periods of time, long-term storage studies have been performed. In 2006 soybean seeds expressing heterologous FanC were placed in plastic zip-lock bags and these bags were locked in a cabinet in a research laboratory. Some of those transgenic seeds were ground to a fine powder and the ground powder was also placed in zip-lock bags for storage in the same laboratory. Ambient conditions in the laboratory were ˜22° C. with ˜50% relative humidity (RH) for the duration of the experiment.

Approximately 8 years following seed harvest and initiation of the experiment, samples of intact seeds and ground powder were removed from storage for FanC protein analysis. PBS buffer and short sonication pulses were used to extract total soluble protein from seed cotyledon chips and ground powder samples. Seed extracts were clarified by centrifugation and a Bradford assay (with BSA as a standard) was used to quantify the total protein in each sample. Five microgram total seed protein samples were separated in 12% SDS-PAGE gels. To aid in the quantification of FanC present in the seed and powder samples, known concentrations of purified recombinant FanC protein (quantification standards) were also included. Separated proteins were transferred to Immobilon P membrane and used in western blot experiments using anti-FanC polyclonal antibodies for detection of the target protein. Results from this experiment demonstrated that FanC remained intact in both, intact seeds and ground powder, following storage for 8 years under ambient laboratory conditions. Importantly, the intensities of the detected FanC bands in the 5 microgram soy protein samples were similar to the intensity of the 20 ng FanC standard indicating that ˜20 ng of FanC protein was present in the 5 microgram sample loaded onto the gel. Thus, the level of FanC protein in 8-year-old seed and powder samples represents ˜0.4% of TSP. This level of protein (˜0.4% of TSP) is identical with the inventors prior measured levels after 1 and 4 years. It is also noteworthy that western blots revealed no sign of FanC protein degradation, even on long X-ray film exposures. Degradation of FanC protein would likely result in products with lower molecular weight than the intact full-length protein (˜18 kDa). These products would be easily separated in 12% SDS-PAGE gels and detected by polyclonal anti-FanC antibodies, as was shown previously in a mock degradation experiment utilizing proteases to create FanC degradation products. To the inventors' knowledge, this is the first demonstration of heterologous protein stability in soybean seeds and ground soybean seed powder following storage for 8 years under ambient laboratory conditions.

The present invention claims that transgenic soybean seeds and ground soybean seed powder compositions containing one or more heterologous proteins involved with the metabolism and/or the deconstruction of lignin, hemicellulose and cellulose can be produced and then stored for years to decades until needed. While the inventors have shown that heterologous protein remains stable for at least 8 years under ambient storage conditions, current best practices for long term storage of soybeans would suggest drying seeds to a moisture content <13% and maintaining a temperature of <22° C. with a relative humidity of <50%. For ideal long term storage of seed powder compositions, they should be placed in vacuum sealed containers prior to storage at <22° C. and RH<50%.

FIG. 2 shows a FanC stability figure or the quantification of heterologous FanC protein in transgenic soybean seeds and ground powder stored for 8 years under ambient laboratory conditions (22° C. and 50% RH). The intact seeds expressing heterologous protein were harvested in 2006 and stored as intact seeds or ground seed powder in plastic zip lock bags for 8 years under ambient laboratory conditions (˜22° C. and 50% relative humidity). Total protein (5 micrograms) from three seed samples (designated A, B, C) and the ground powder (D) were separated in 12% SDS-PAGE gels prior to detection. Non-transgenic (WT) protein (5 ug) was included as a negative control. Known amounts of purified FanC (derived from E. coli) were included as a positive control. The 8-year-old samples contain 20-30 ng FanC per 5 ug sample indicating >0.4% TSP. The absence of degraded FanC further demonstrates the stability of this heterologous protein and potential for long term storage of transgenic seeds and seed powder.

### Example

### Solubility of Heterologous Proteins in Around Soybean Seed Compositions Defined by Seed Powder Particle Size

As mentioned above, there is a direct correlation between protein extractability and seed powder particle size. In the present invention, one or more soy powders containing enzymes capable of deconstructing lignin, hemicellulose and cellulose are added to a liquid substrate. Soy protein containing the deconstructing enzymes are then solubilized and begin to carry out their specific enzymatic functions. Particle size is important for rapid and efficient solubilization of soy protein. In general, smaller seed powder particles will elute more protein than an identical weight of larger powder particles if aqueous volume and time are kept constant.

After soybean seeds are ground, the powder can be passed over a series of sieves or screens to separate the powder particles according to size. Standard sieves, screens and filters are available to create various compositions of seed powder containing pre-determined particle sizes.

To demonstrate the importance of ground seed particle size as it relates to this invention, sifting pans can be purchased from Sigma and mesh filters with various cut off sizes (e.g. 20 mesh, 30 mesh and 50 mesh) can be purchased from Bellco Glass Inc. The mesh screens are held in place by a retaining ring located at the bottom of the sifter pan and tightened with a specialized key. Transgenic seeds expressing either human thyroglobulin protein (hTg) or S. aureus mutant enterotoxin B (mSEB) were first ground to a coarse powder in a Mr. Coffee coffee grinder using 1 second pulses. Seed pieces with an average diameter of ˜6000 microns were collected and served as the coarsest of the ground powders tested. The remaining powder was ground further using 1 second pulses and seed pieces with an average diameter of ˜4000 microns were collected. The remaining seed mixture was ground to a fine powder and separated according to particle class size with the aid of sifting pans witted with various mesh screens. The powders were first sifted over a 20 mesh screen. The 20 mesh screen (Bellco Glass Inc.) has a particle size cut off of 860 microns. Thus, particles that did not pass through the 20 mesh screen were >860 microns in diameter and <4000 microns in diameter while those that passed through the 20 mesh screen were <860 microns in diameter. The particles <860 microns in size were then passed over the 30 mesh screen which has a particle size cut off of 520 microns. Thus, particles that did not pass through the 30 mesh screen were >520 microns in diameter but <860 microns in diameter. The particles <520 microns in size were further passed over the 50 mesh screen which has a particle cut off size of 280 microns. Therefore, particles that passed through the 50 mesh screen were <280 microns in diameter while those that did not pass through the 50 mesh screen were >280 microns in diameter but <520 microns in diameter. Table 8 below summarizes the different particles that were collected using the various mesh screens.

TABLE 8

| Method | Particle size | |
|---|---|---|
| Ground and hand selected | ~6000 | microns |
| Ground and hand selected | ~4000 | microns |
| 20 mesh cutoff | 860-4000 | microns |
| 20 mesh + 30 mesh trap | 520-860 | microns |
| 30 mesh + 50 mesh trap | 280-520 | microns |
| 50 mesh pass through | <280 | microns |

To evaluate solubility of the various particle size classes, 100 mg of each powder composition was added to 10 ml of water containing TE (10 mM Tris, pH 8.0, 1 mM EDTA) and incubated at 23° C. for either 1 minute or 30 minutes with gentle inversion. At the respective time points, the aqueous mixtures were clarified by centrifugation at 16,000×g for 10 minutes at 4° C. Soluble protein extracts (12 μl volume) were mixed with 3×SDS sample buffer and loaded onto SDS-PAGE gels. The 1 minute and 30 minute hTg samples were run together on a 5% SDS-acrylamide gel while the 1 minute and 30 minute mSEB samples were run together on a 10% SDS-acrylamide gel. Following separation for 1-2 hours at 120 V the separated proteins were transferred in 10 mM CAPS buffer (pH 11) to Immobilon-P membrane (Millipore, Bedford, Mass., USA). Western analyses was performed using either rabbit polyclonal anti-hTG serum (1:5000) or rabbit polyclonal snit-mSEB serum (1:5000) as the primary antibody followed by goat anti-rabbit IgG conjugated with horseradish peroxidase as the secondary antibody (Cell signaling). Immunodetection was performed using the Supersignal West Pico Chemiluminescent Substrate Kit (Pierce, Rockford, Ill.). The western results show that particles with a diameter of <280 microns were the most efficient in solubilization of heterologous protein at both the 1 minute and 30 minute time points. This was true for compositions containing either hTg or mSEB. Comparison of all hTg compositions suggested that similar levels of heterologous protein were solubilized following the 1 minute and 30 minute incubation time points. However, comparison of the mSEB compositions showed that greater levels of mSEB protein were present following solubilization for 30 minutes. This is most noticeable in compositions D, E and F (see FIG. 2). This result could be due to the fact that mSEB is a relatively small protein and therefore may continue to elute over time. Alternatively, the biochemical properties of mSEB protein, the composition of the aqueous buffer (e.g. salt concentration, pH, etc.) or both may have played a role in the rate of solubilization. Regardless, the solubilization of heterologous proteins after only 1 minute in aqueous buffer is impressive.

FIG. 2 shows Western blots showing the solubility of heterologous hTG and mSEB in various seed powder compositions with particle sizes ranging from <280 microns to ˜6000 microns in diameter.

Protein concentrations were determined with the Bradford Reagent (Bio-Rad Laboratories, Hercules, Calif.) using bovine serum albumin (BSA) as a standard and these concentrations were used to calculate the absolute amount of soy protein solubilized in each particle composition. The addition of 100 mg of powder to a 10 ml liquid volume resulted in minimal losses as 9.9 ml of the original 10 ml aqueous volume was recovered. Soy protein concentrations calculated as mgs of protein per ml aqueous solution were multiplied by 9.9 ml to obtain total extracted soy protein shown below. From these calculations there is a clear trend between particle size and the absolute amount of soluble protein recovered in solution which underscores the importance of particle size for maximal protein solubilization. For the hTG samples, 22.9 mg of soy protein was recovered as soluble protein from the <280 particle composition following a 1 minute incubation. Since soybean seeds comprise ˜40% protein, it can be assumed that 100 mg of starting mass contained ˜40 mg of protein of which 22.9 mg was recovered in 1 minute. This translates to 57% solubilization of gross soy protein present in this composition. In contrast, the hTg composition with the largest particles solubilized only 1.1 mg of soy protein, or 3% of the gross protein present in the composition. Similar trends were also observed in the mSEB compositions as the greatest recovery of soluble protein was 17.8 mg for the <280 micron composition (45% of the total gross protein) while the least recovery was 1.1 mg for the ˜6,000 micron composition (3% of total gross protein). Thus, there was a 15-20-fold difference in solubility between the largest (˜6000 microns) and smallest (<280 microns) powder particles tested in this experiment.

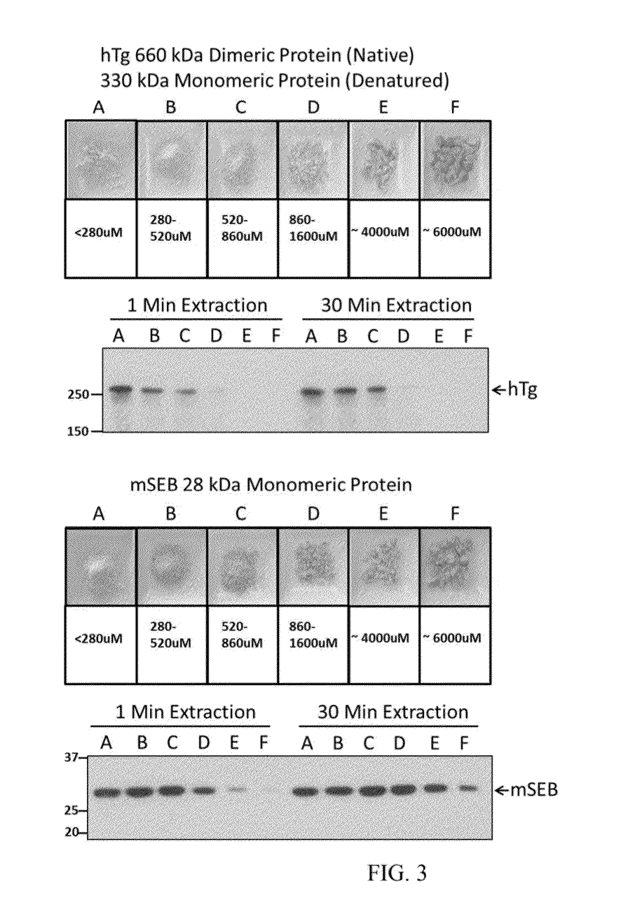

FIG. 3 shows a calculations figure. In FIG. 3, larger meshes (smaller micron sizes) are available from Bellco Glass Inc. and the use of such meshes allow for additional separation beyond the <280 micron class. For example, an 80 mesh screen has a 190 micron particle cut off; a 100 mesh screen has a 140 micron particle cutoff; a 200 mesh screen has a 74 micron particle cutoff; a 300 mesh screen has a 46 micron particle cutoff; a 400 mesh screen has a 38 micron particle cutoff and a 500 mesh screen has a 25 micron particle cutoff. These and many other meshes, filters, sieves and screens are available to separate seed powder particles according to size.

In FIG. 3, transgenic soybean seeds expressing either heterologous human thyroglobulin protein (hTG) or S. aureus mutant enterotoxin B (mSEB) protein were ground to a powder and particles were separated using mesh screens with different particle size cutoff. Photographs of the various compositions, along with corresponding particle size diameters, are labeled A-F. Samples (100 mg) of each composition were added to 10 ml aqueous solutions (TE buffer, pH 8) and gently inverted for 1 minute or 30 minutes. Equal amounts (10 ul) of solubilized samples were separated in SDS-PAGE gels and subjected to western blot analysis. Western blots were probed with either anti-hTG antibodies or anti-mSEB antibodies to visualize the respective heterologous protein. The numbers on left indicate size and position of molecular mass standards (expressed as kDa) and arrows at right indicate the detected heterologous protein. hTG is a homodimeric protein but migrates as monomeric protein under SDS denaturing conditions shown here. There is a clear correlation between particle size and dissolution of heterologous protein. Compositions containing particles with diameters <280 microns were most efficient at protein dissolution while compositions containing particles with diameters >860 microns were the least efficient at protein dissolution.

While the above example of seed grinding and particle size determination was carried out at laboratory scale, the present invention contemplates using similar particle sizing processes carried out on a larger scale. Grinding machinery and large sieves, screens, filters and meshes are available for such scaled up processes and the inventors believe that there would be few problems or issues introduced in the scale-up process that would prevent ground soybean seed powder particles of known size from being collected.

### Example

### Examples of Soybean Seed Powder Compositions and Solubility of Heterologous Proteins in Compositions

One aspect of this invention is flexibility which allows the mixing of one or more soybean seed powders containing one or more enzymes to create custom powder compositions that can be tailored for a variety of specific reactions. For example, one may want to create a seed powder composition containing 60% of a specific endocellulase and 40% of a specific exocellulase. Alternatively one may want to create a seed powder composition consisting of 60% lignin peroxidase, 30% versatile peroxidase and 10% Xylanase. The expression of various enzymes in soybean seeds will allow for an increase in the number of novel compositions that can be created. Such seed powder compositions could then be added to specific reaction where it would be anticipated that the various enzymes comprising the composition would solubilize within a relatively short period of time (e.g. minutes).

To demonstrate the flexibility of this invention in creating custom soybean powders expressing one or more heterologous proteins, three separate powder compositions were created utilizing three different transgenic soybeans lines expressing different heterologous proteins. One transgenic soybean line expresses heterologous S. aureus mutant SEB protein (mSEB, ˜28 kDa) while a second transgenic soybean line expresses heterologous fusion protein containing human myelin basic protein (hMBP, ˜75 kDa) and a third transgenic soybean line expresses heterologous human thyroglobulin protein (hTG, ˜660 kDa dimeric protein under native conditions and ˜330 kDa monomeric protein under denaturing conditions). Each of these three transgenic lines expresses the respective heterologous protein at levels >1% of total soluble protein (TSP). Transgenic soybeans from each of the three lines were ground to a powder with particle sizes of <280 microns. Three compositions were prepared by mixing different proportions of each of the above powders. Composition A contained 10% mSEB powder, 30% hMBP powder and 60% hTG powder; Composition B contained 30% mSEB powder, 60% hMBP powder and 10% hTG powder; Composition C contained 60% mSEB powder, 10% hMBP powder and 30% hTG powder.

A 100 milligram sample of each powder composition was added to 10 ml of TE buffer (pH 8) and the buffer solution was gently inverted for either 1 minute or 30 minutes. At the stated time points, the aqueous solutions were clarified by centrifugation at 16,000×g for 10 minutes at 4° C. and total protein was quantified by the Bradford assay using BSA as a protein standard. Equal amounts (12 microliters) of extracted protein from Composition A, B and C at each time point were loaded in triplicate onto 4-15% SDS-PAGE gradient gels. Separated proteins were transferred in 10 mM CAPs buffer (Sigma, St. Louis, Mo.) to Immobilon-P membrane (Millipore, Bedford, Mass.) and blocked overnight with PBS containing 5% nonfat powdered milk (block solution). The Immobilon membrane was then divided into three identical panels, each containing a replicate of the different compositions. One panel of the membrane from each time point was incubated with rabbit serum containing anti-mSEB polyclonal antibodies in fresh block solution for 16 hours at 4° C. The second and third panels were incubated with rabbit serum containing anti-MBP-fusion protein and anti-hTG polyclonal antibodies under identical conditions. All membranes were washed three times for 10 minutes each at room temperature with PBST and then incubated for 45 minutes with a goat anti-rabbit immunoglobulin antibody conjugated with horseradish peroxidase (Cell Signaling Technology) in block solution. Following three additional washes with PBST, immunodetection was carried out using the SuperSignal West Pico Chemiluminescent Substrate kit (Pierce, Rockford, Ill.) and bands were visualized with BioMax film (Kodak, Rochester, N.Y.).

Analysis of X-ray films verified that all three heterologous proteins were soluble in TE buffer after only 1 minute of incubation with gentle inversion. Furthermore, each protein was solubilized in proportion to the percentage of protein present in each composition. For example, soluble protein from powder composition “C” contained the most mSEB protein (60%) relative to the other compositions and the least hMBP fusion protein (10%) relative to the other compositions, while soluble protein from powder composition “B” contained the most hMBP fusion protein (60%) and the least hTG protein (10%) relative to the other compositions. Similar ratios of the three solubilized heterologous protein were also detected in protein samples that were incubated with gentle inversion for 30 minutes. These results demonstrate that multiple heterologous proteins comprising in a single soybean seed powder composition are rapidly solubilized when added to an aqueous solution.

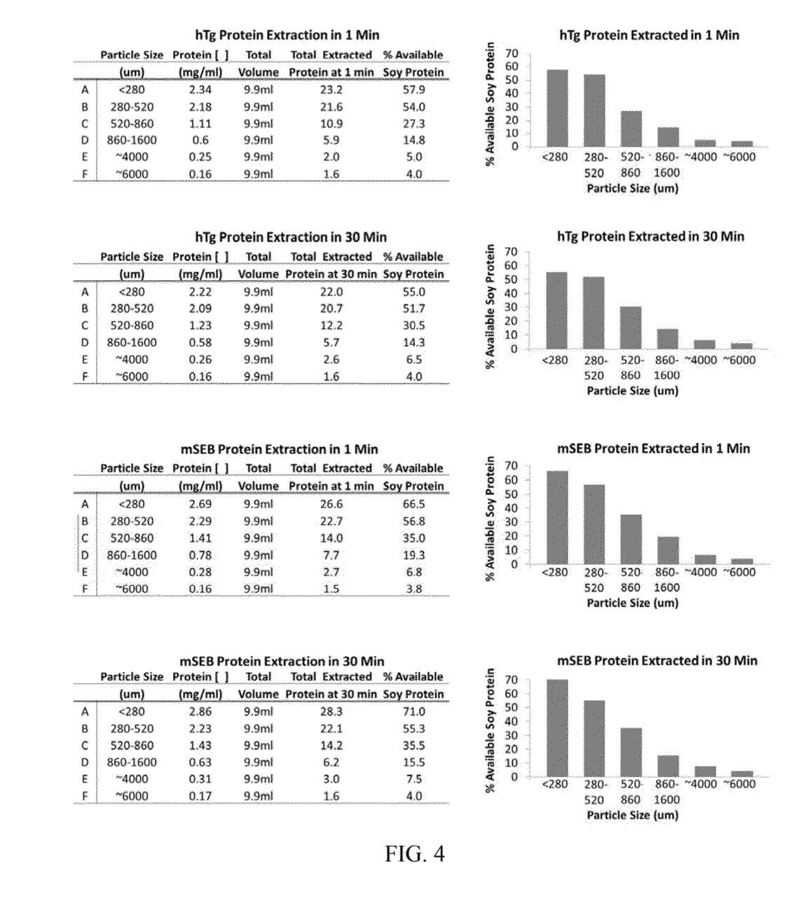

FIG. 4 shows the protein concentrations of samples shown in FIG. 3, which were determined using the Bradford assay with BSA as a protein standard. The total amount of solubilized bulk soy protein was calculated by multiplying protein concentration by the total volume of recovered aqueous solution (9.9 ml). The total amount of solubilized soy protein was calculated by dividing the mass of recovered protein by 40 gm (the assumed mass of protein in a 100 mg seed powder sample) and multiplying by 100 to obtain percentage. These percentages are shown as histograms (right panels). Compositions with particles <280 in diameter solubilized 58-67% of available bulk protein within 1 minute, while compositions with particles averaging 6000 microns in diameter solubilized only 4% of the available protein. Thus, a clear trend was observed between particle size and total protein solubilized, with greater dissolution levels skewed heavily towards smaller particles. Longer incubations (e.g. 30 minutes) did not result in significant increases of recoverable soluble protein.

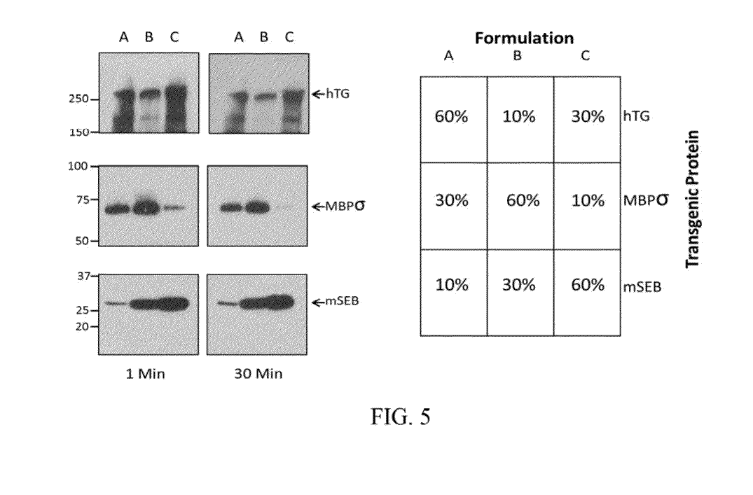

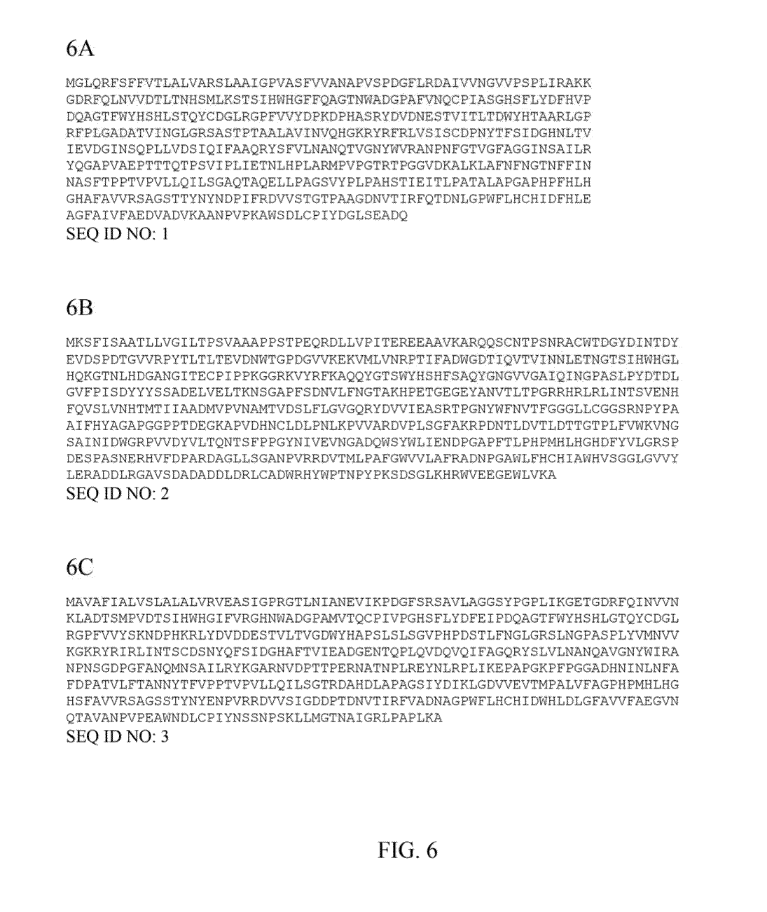

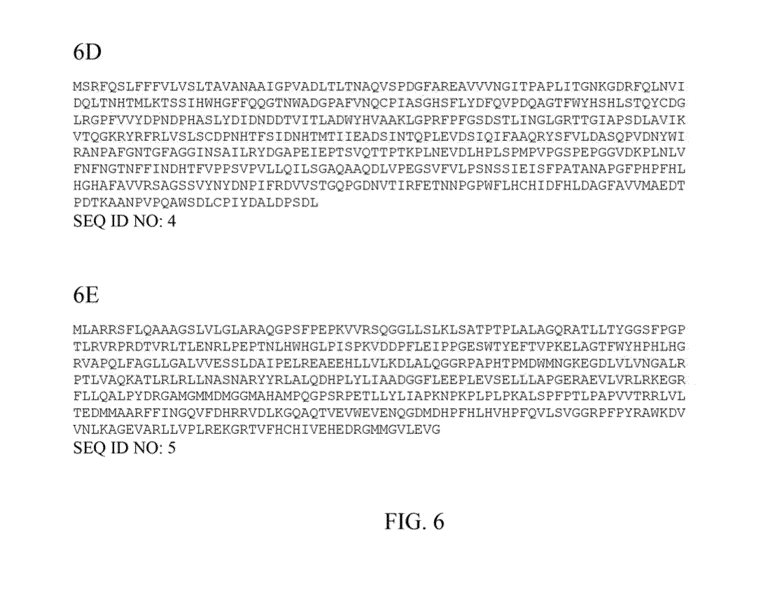

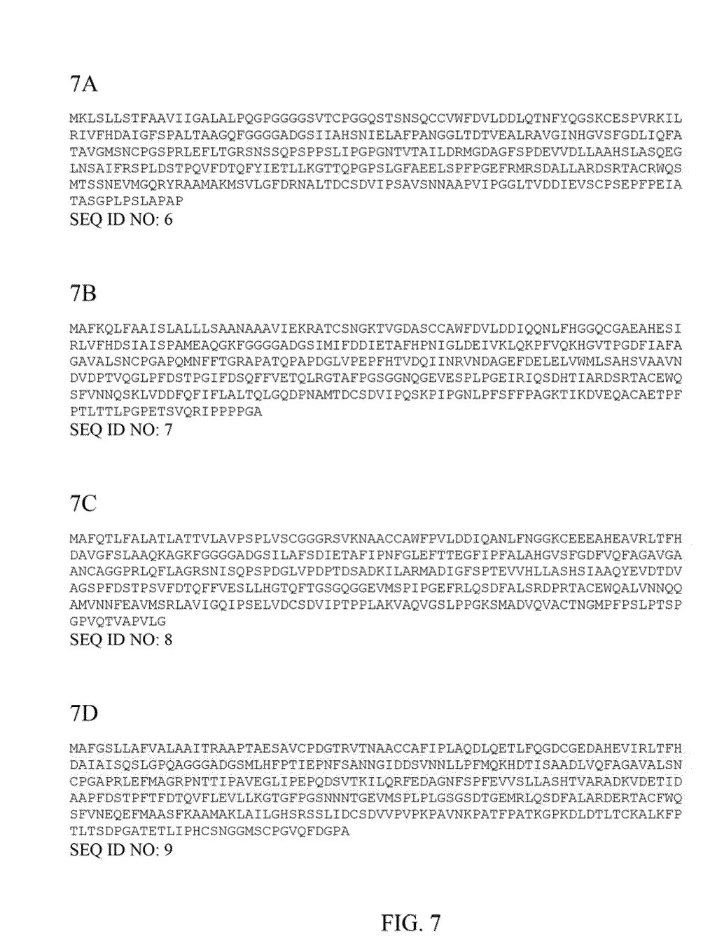

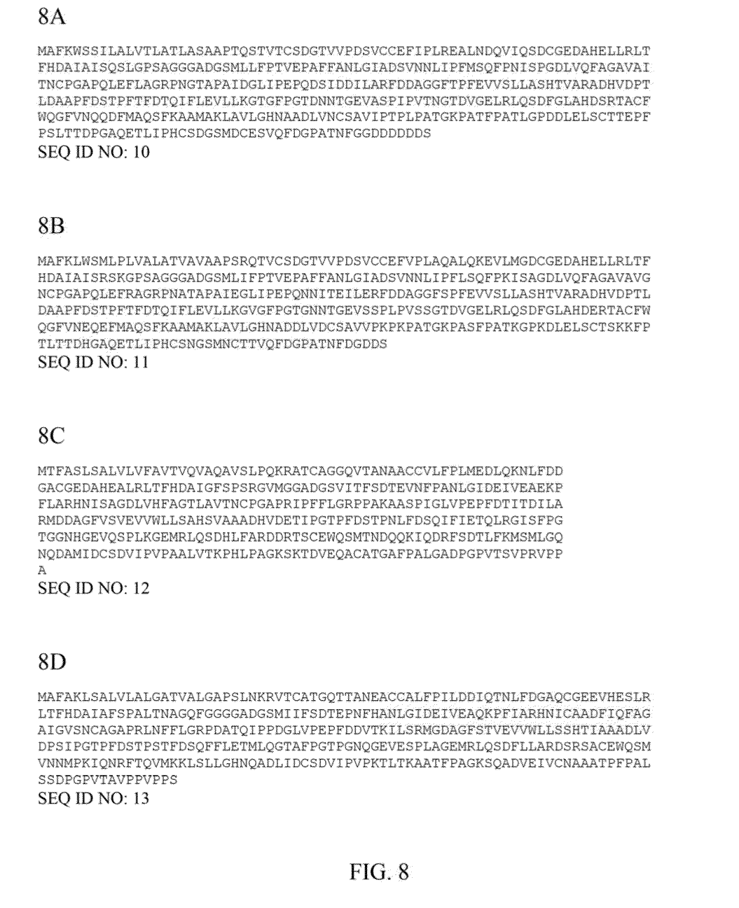

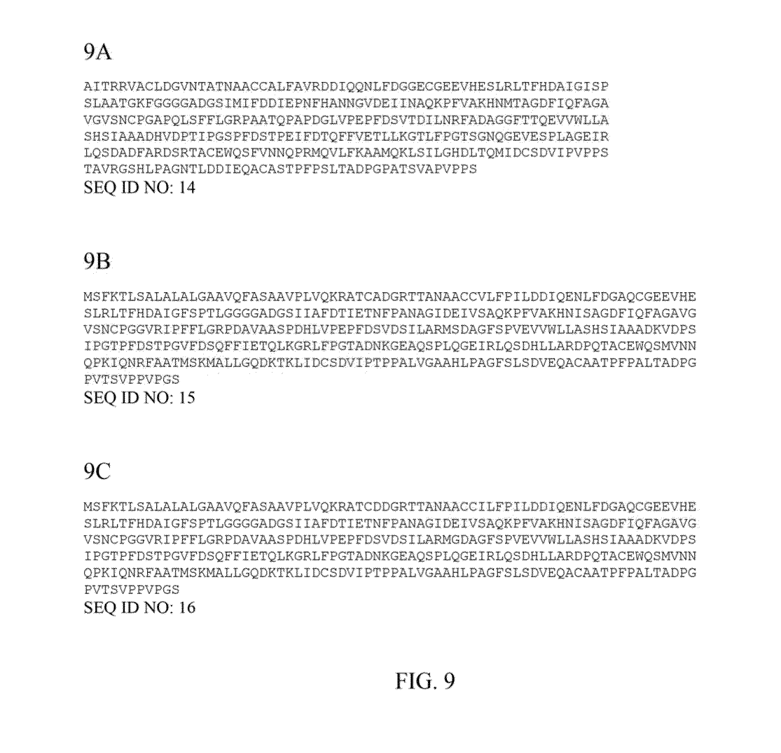

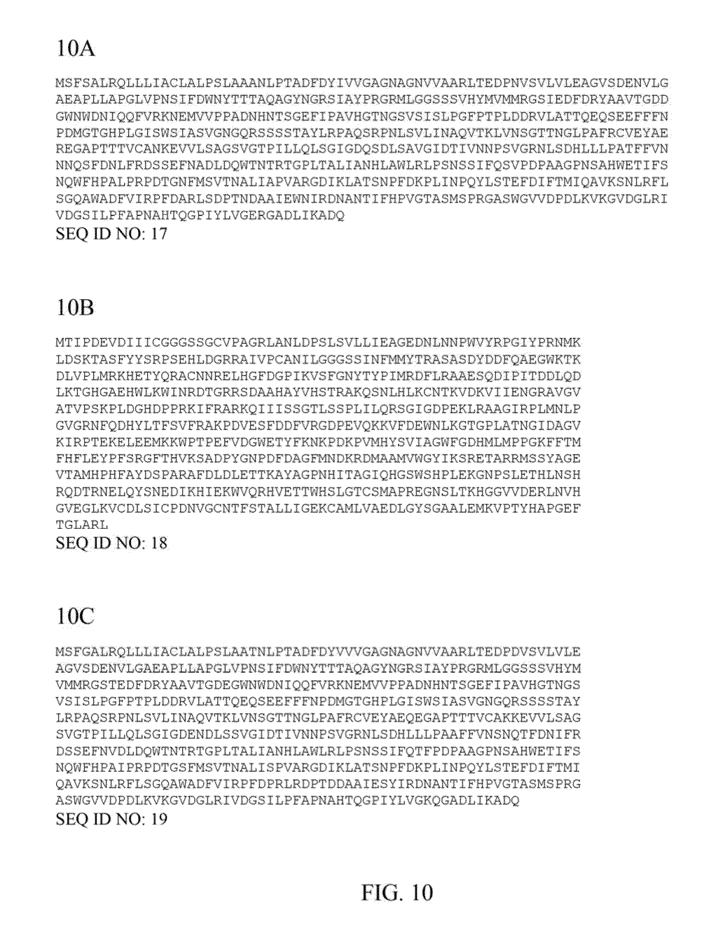

FIG. 5 shows the dissolution of heterologous protein, which was characterized in compositions containing differing amounts of transgenic seed powder. Transgenic seeds expressing human thyroglobulin (hTG) protein, human myelin basic protein (hMBP) fusion protein and S. aureus mutant enterotoxin B (mSEB) protein were ground and sifted to a particle size <280 microns in diameter. The powders were then mixed in different proportions to obtain Compositions A, B and C (shown in the right panel). Samples (100 ug) of each composition were added to 10 ml aqueous solutions (TE, pH 8) and inverted for 1 minute. Equal volumes (12 ul) of each sample were loaded onto 4-15% SDS-PAGE gradient gels and subjected to western blot analysis using appropriate antibodies for detection of the various heterologous proteins present in each composition. Western blot results (left panels) show that the relative level of heterologous protein in solubilized samples correlates with the relative amounts of particular powders used to create the compositions. For example, the greatest level of mSEB and lowest level of hMBP was observed in Composition C which contained 60% powder derived from seeds expressing the mSEB protein and 10% powder derived from seeds expressing the hMBP protein. These results demonstrate that multiple heterologous proteins can be solubilized simultaneously from complex compositions created by mixing different amounts of powders together. These results also demonstrate that solubilization of heterologous proteins is proportional to the amount of powder containing that protein in a given composition. It is anticipated that the solubilization of other heterologous proteins (e.g. enzymes involved with deconstruction of lignin, hemicellulose and cellulose) will also be solubilized in proportion to levels present in mixed powder compositions.

### Other Non-Limiting Examples and Implementation of the Present Invention

### Expression of the Full Length Human Thyroglobulin Gene in Transgenic Soybean Seeds

Human thyroglobulin (also referred to herein as hTg) is encoded by an 8.3 kb mRNA species encoding 2767 amino acids with a molecular weight of the mature monomer being over 300,000 daltons. Mature human thyroglobulin is also glycosylated by post translational modification. Thus, thyroglobulin is a very large protein which presents some significant challenges when trying to express this protein using traditional expression systems (e.g. E. coli), and it has been difficult (if not impossible) to accomplish. Improper folding of thyroglobulin results in its degradation and has been a major hurdle to overcome. Yeast has also been used in the past as a recombinant expression system for heterologous proteins. However, variations in glycosylation in yeast have been an obstacle that has often led to decreased yields and to the inventor's knowledge, yeast has not been capable of expressing thyroglobulin. One function of the thyroid gland is to store thyroglobulin. In this sense, the thyroid gland is a storage organ. Soybean seeds also function to store proteins needed for germination. So soybean seeds can also be considered storage organs. Soybean storage proteins are large and complex, consisting of subunits from major classes of soybean storage proteins such as the glycinins and conglycinins. Assembly of subunits from major class storage proteins result in the large complexes present in soybean seeds. Since soybean seeds are natural storage organs and support high levels of large and complex storage proteins, soybean would appear to be an ideal host for the expression and long term storage of large, complex and traditionally difficult-to-express proteins.

### Design of Thyroglobulin Nucleotide Sequence

A soybean compatible version of full-length human thyroglobulin was synthesized by GeneArt (Life Technologies). The protein sequence encoded by the synthetic gene is identical to that of the human protein sequence. However, it was necessary to modify the nucleotide sequence, while keeping the encoded amino acids the same, to permit the soybean seeds to express optimal levels of this protein.

Because human Thyroglobulin (hTg) is made in the endoplasmic reticulum (ER), and is heavily glycosylated, and is secreted, it was postulated that the synthetic version should also be translated by the rough ER (e.g. secretory pathway) but not retained there. An assumption is that the endogenous leader should target hTg to the proper location for translation, so the synthetic gene was designed with an intact leader sequence. It was also expected that the leader would be cleaved by the soy plant machinery. It was postulated that no KDEL (lys-asp-glu-leu) sequence (the most common endoplasmic retention sequence) should be required, as one is not present in the wild type human version. It was also postulated that the cloned synthetic gene could be placed downstream of the 7S promoter and fused to a translational enhancer sequence (e.g. TEV, Tobacco Etch Virus). To aid in purification, it was postulated adding a His tagged linker (and thus, the His tag was added) at the C-terminus. Other amino acid sequences to aid in purification (and placed at either the N-terminus or C-terminus) were contemplated, such as GST tags, FLAG tags, HA tags, and MYC tags. It is also postulated that biotin-strepavidin chemical tags can be used to aid in the purification process. The amino acid sequence of the expressed gene was cross checked against the known sequence. The inventors postulated and used 5′ NcoI and 3′ XbaI for cloning. The inventors did not use the TGA for the stop codon as the inventors knew that the overlapping methylation would prevent XbaI digestion. Moreover, the wobble position of each codon was often changed to make the sequence more amenable to expression in soybean. Generally, the nucleotide sequence that is optimized for soy tends to contain a lower GC content than the corresponding wildtype human thyroglobulin.

### Synthesizing Nucleotide Sequence

The nucleotide sequence was synthesized using standard nucleotide synthetic techniques by GeneArt (Carlsbad, Calif.). A comparison between the open reading frame of wildtype thyroglobulin and the nucleotide sequence used for the soybean transformed thyroglobulin revealed a sequence homology in the nucleotide sequences in 6325 of the 8311 nucleotides for a sequence homology percentage of 76%. The synthetic sequence was entered into a DNA translation program to verify the presence of a single, large open reading frame.

### Transformation

The synthetic hTG gene was designed and engineered as above to contain a native signal sequence, a GC content representative of plant systems, and codons that were optimized for expression in the Glycine max system. The synthetic hTG was subcloned downstream of the soybean 7S (beta-conglycinin) promoter resulting in the binary vector pPTN-hTG. In addition to the hTG (synthetic human thyroglobulin gene), the expression cassette was designed to contain P-7S (the soybean beta-conglycinin promoter), TEV (tobacco etch virus translational enhancer element), and T-35S (cauliflower mosaic virus terminator element). The plant selection cassette contained P-nos (nopaline synthase promoter), Bar (phosphinothricin acetyltransferase gene for plant selection), and T-nos (nopaline synthase terminator element). Both cassettes were placed between the RB (right border sequence) and LB (left border sequence), in a binary vector that contained the aad A region (streptomycin resistance gene for bacterial selection).

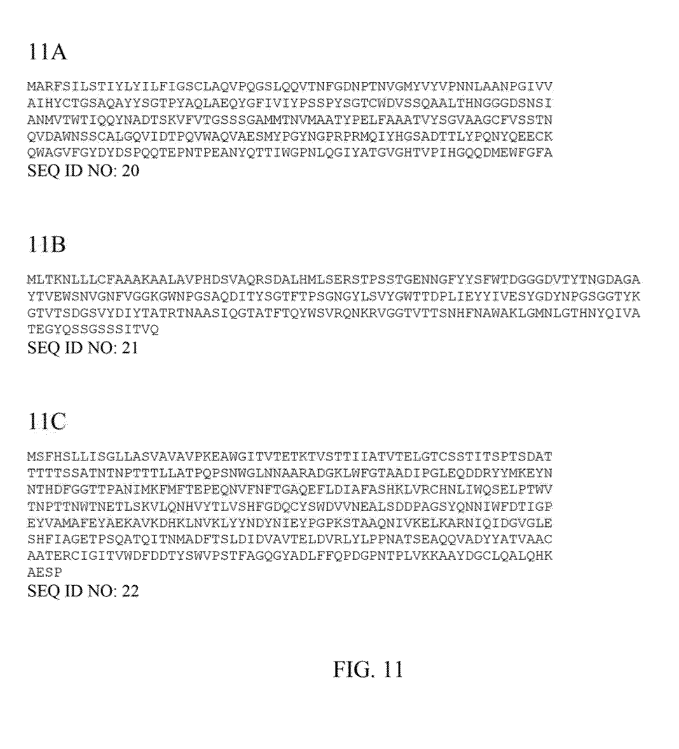

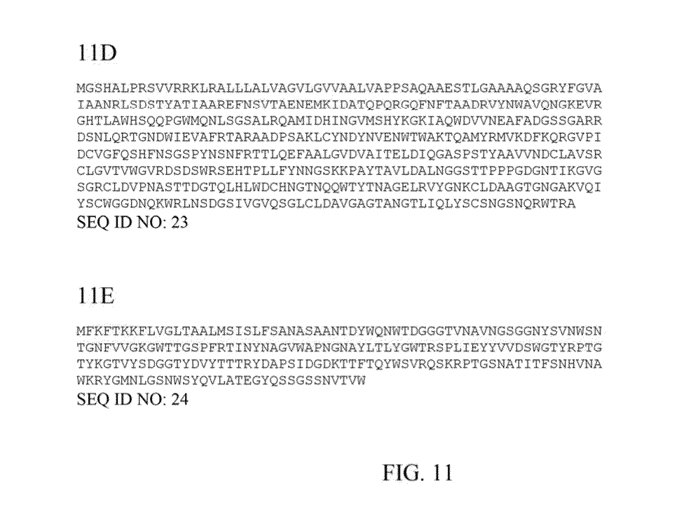

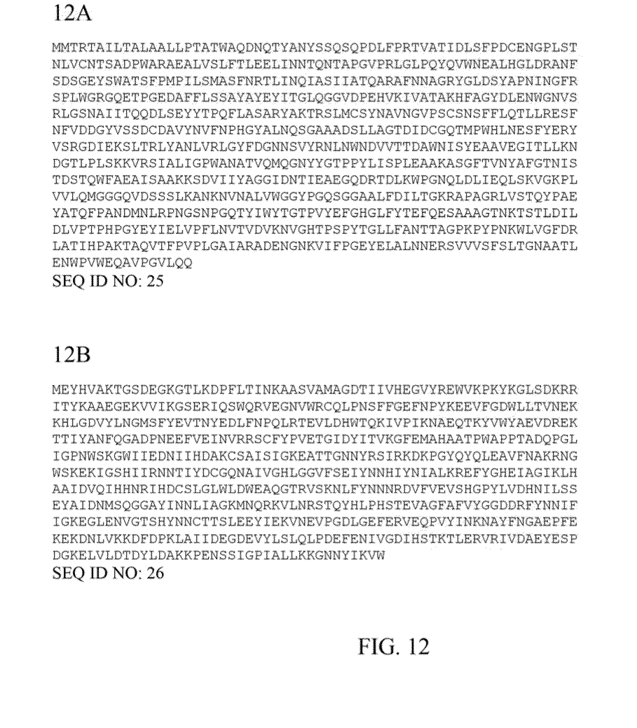

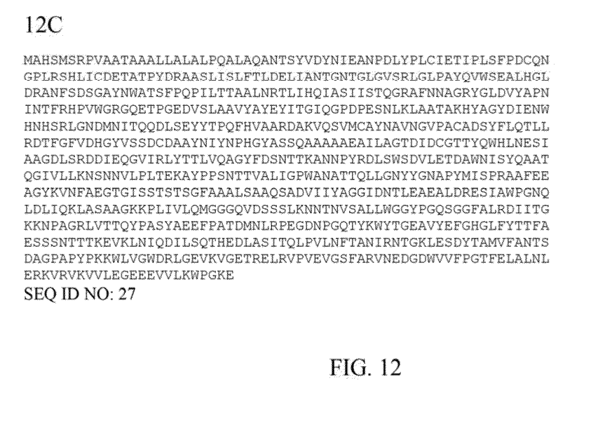

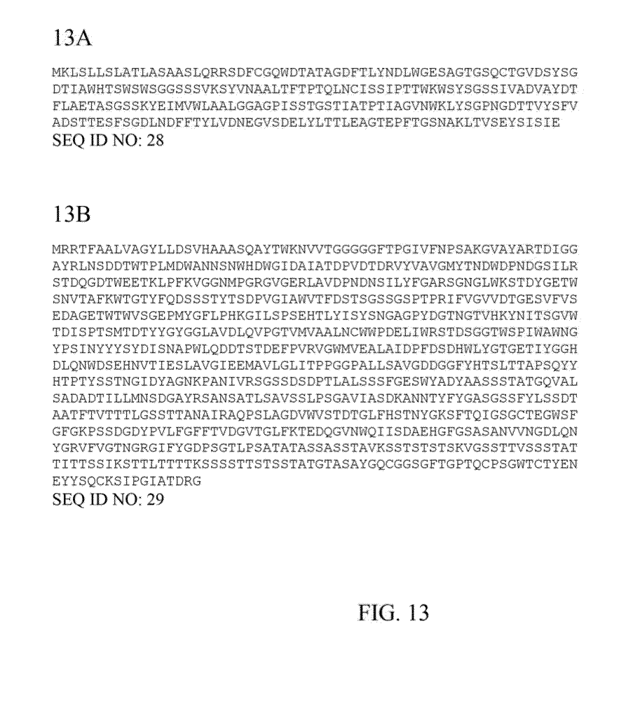

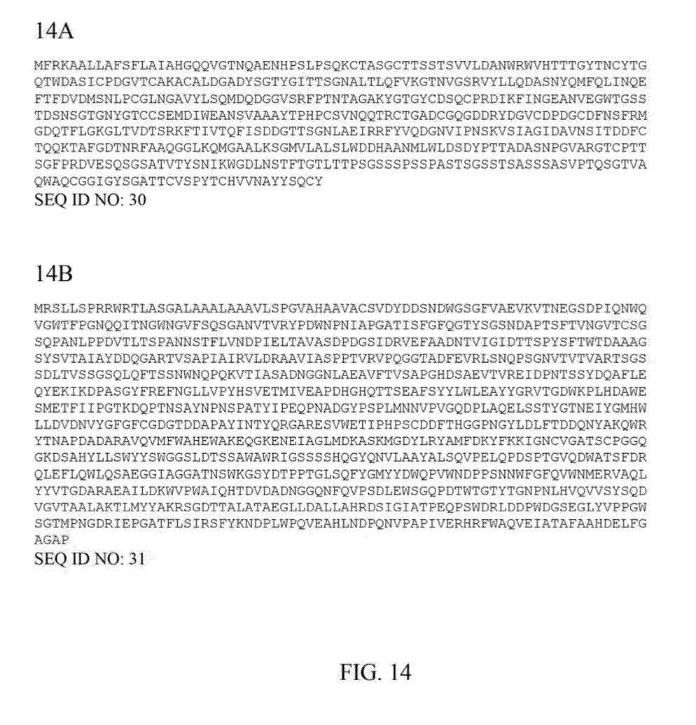

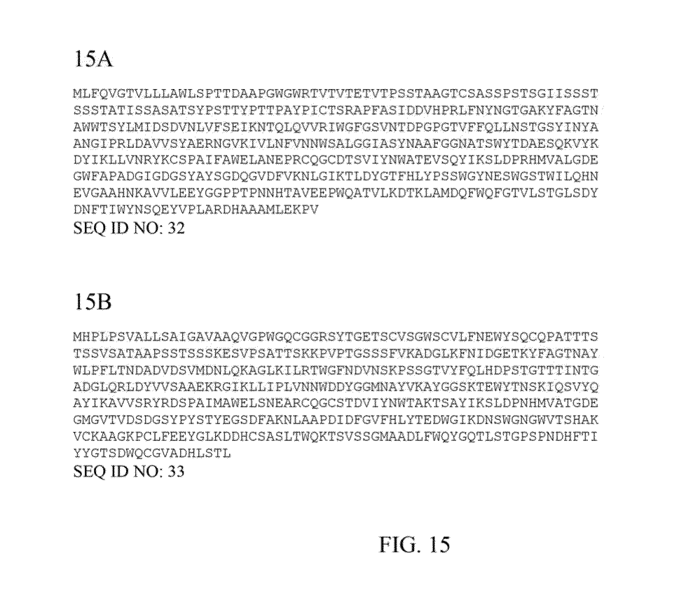

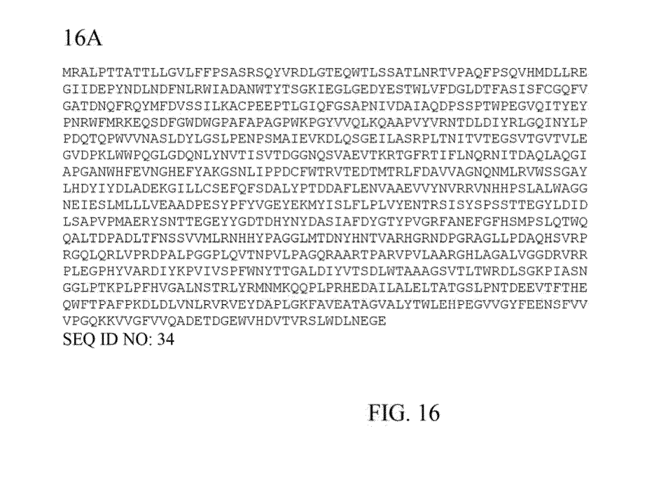

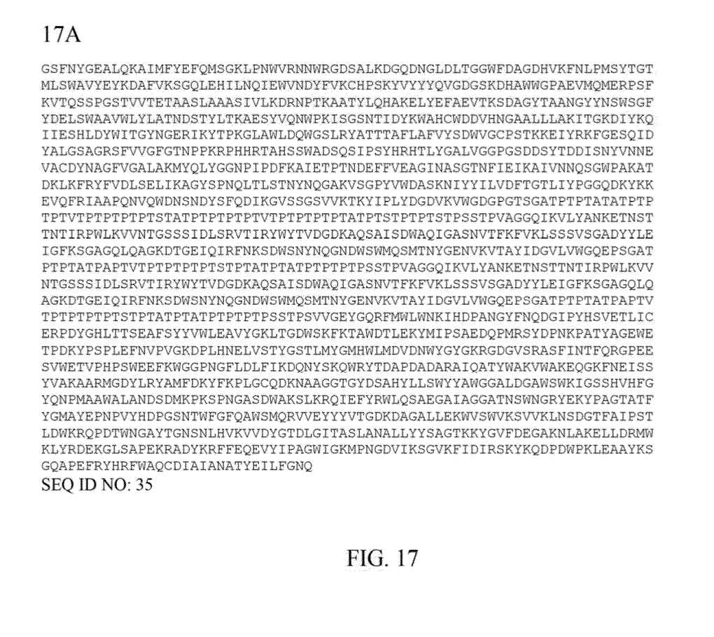

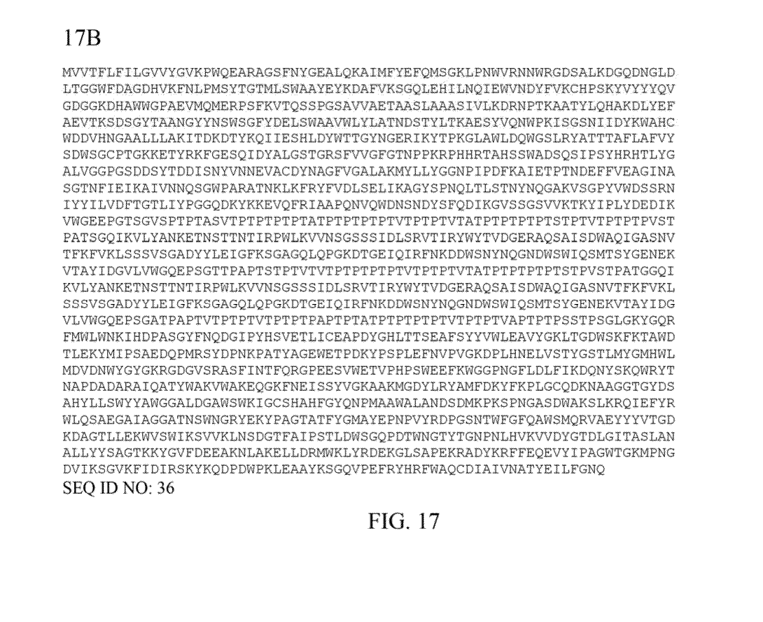

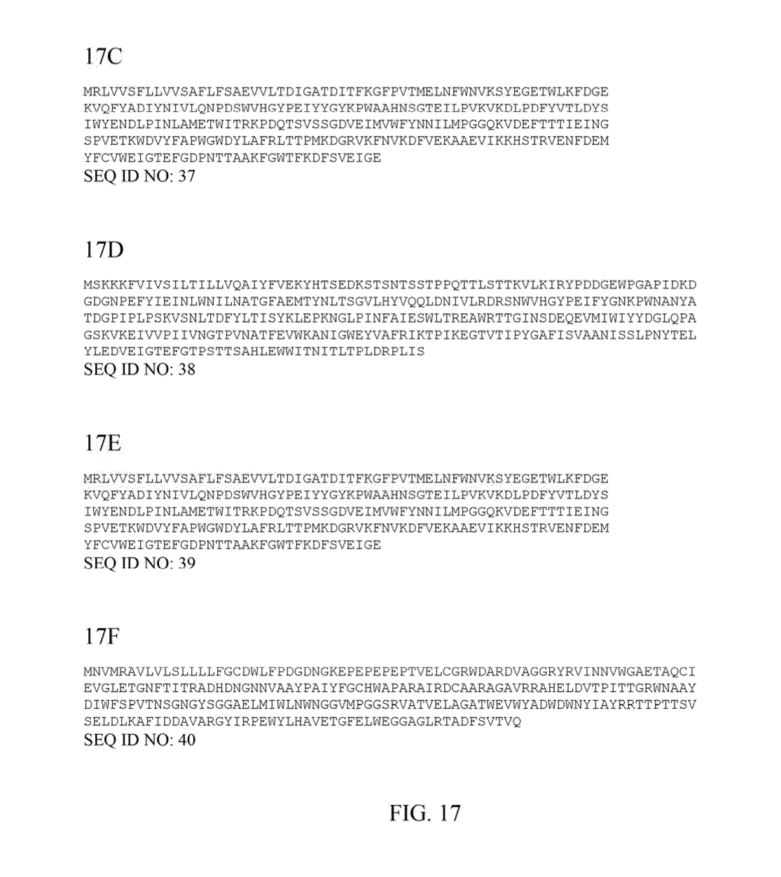

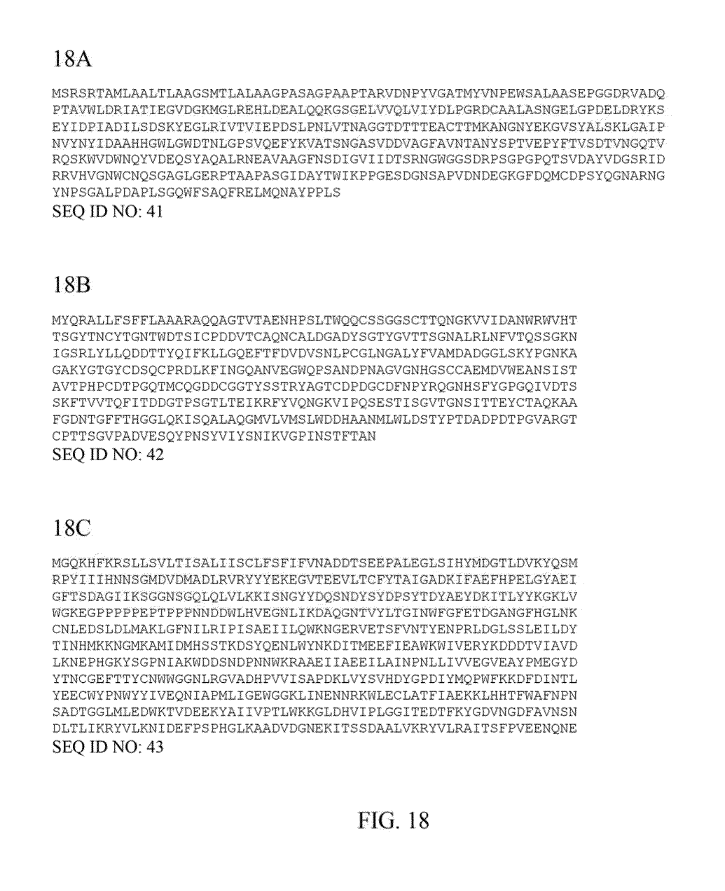

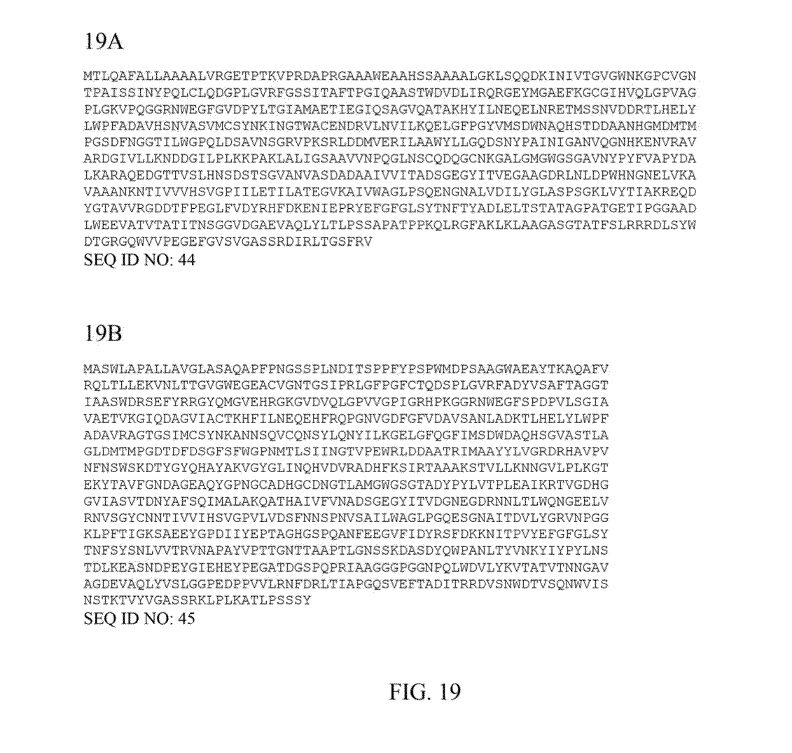

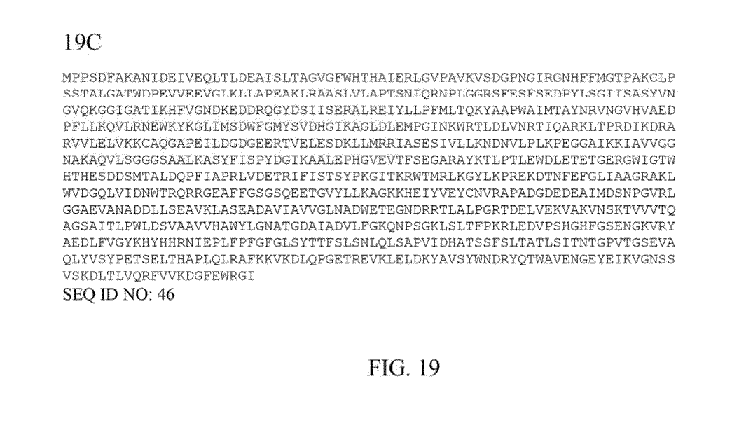

Soybean transformation using the Agrobacterium-mediated half seed method was performed as described in Paz et al (XX). Briefly, half-seed explants (Glycine max) were dissected and inoculated with Agrobacterium suspension culture (strain EHA 101 carrying various binary vectors). The inoculated explants were placed adaxial side down on cocultivation medium at 24° C. and under 18:6 photo period for 3-5 days. After cocultivation, explants were cultured for shoot induction and elongation under glufosinate selection (8 mg/L) for 8-12 weeks. Agrobacterium-mediated transformation resulted in five independent T0 lines designated 77-3, 77-4, 77-5, 77-7 and 77-12. Phenotypically, T0 parent plants as well as T1 and T2 progeny plants all appeared similar to wild type nontransgenic control plants with respect to leaf color, growth habit and relative seed yield. 60-day old transgenic (line 77-5) and WT (control) plants are shown in FIG. 11. To monitor for expression of the glufosinate herbicide selectable marker, TI and T2 plants were sprayed with Ignite 280 SI, herbicide (Bayer CropScience, RTP, NC) at a concentration of 80 mg/l for a total of three times (days 1, 3, and 5). Plants with visible chlorosis similar to that observed in nontransgenic plants were scored as negative for resistance to the herbicide and discarded, while positive plants were taken to maturity. Plants known to be resistant to phosphinothricin were included as a control for spray concentration and application.

Individual T1 seeds were harvested from several surviving plant lines, and were screened for the presence of human thyroglobulin. First, genomic DNA was isolated from individual T1 seed shavings and from control seeds. In particular, genomic DNA was prepared from cotyledon tissue using the Maxwell 16 Instrument and Maxwell Tissue DNA Purification Kit (Promega, Madison, Wis.). Soybean genomic DNA (100 ng), specific primers for detecting hTg and specific primers for detecting the vsp gene (serving as an internal control), and dNTPs were mixed with GoTaq Flexi DNA polymerase and buffer (Promega Corp., Madison. WI) according to the manufacturer's directions. Following an initial denaturation cycle (5 minutes at 94.degree. C.) the reactions were subjected to 38 cycles comprised of denaturation (30 seconds at 94.degree. C.), annealing (45 seconds at 58.degree. C.) and extension (60 seconds at 72.degree. C.). PCR products were visualized in 1.0% agarose gels stained with ethidium bromide. Genomic DNA was isolated from a nontransgenic seed and served as a negative control. The plasmid DNA (pPTN-hTG) used for soybean transformation served as a positive control for the PCR reaction. The presence of a 659 bp product indicated the presence of the hTg present on the integrated T-DNA. The presence of the 325 bp product served as an internal control for the presence of DNA in reactions that did not contain the 659 bp product (e.g. nontransgenic seeds arising from segregation of the alleles transformed with T-DNA).

### Soybean-Derived Thyroglobulin Protein is Recognized by Commercially Available ELISAs

To begin to evaluate thyroglobulin protein expression by transgenic soybean seeds, two different commercially available ELISAs and one designed by the inventors were used. All of these ELISA use pairs of antibodies in a capture/detection format.

In the first ELISA, the total soluble protein was isolated from 6 different individual TI seed shavings from 5 different transgenic soybean lines. In particular, seed chips (˜10 mg of cotyledon tissue) were resuspended in 150 μl of phosphate buffered saline (PBS) and sonicated for 30 seconds using a Vibra-Cell ultrasonic processor (Newton, Conn.). Samples were clarified from insoluble debris by centrifugation at 16,100.×g at 4° C. Total soluble protein was quantified with the Bradford Reagent (Bio-Rad, Hercules, Calif.) using bovine serum albumin (BSA) as a standard. These soluble protein isolates were then assayed several ways using two commercially available ELISA. One ELISA from Orgentec (Orgentec. Mainz. Germany) was used to detect the presence of human thyroglobulin. The commercially available ELISA from Orgentec uses polyclonal anti-human thyroglobulin antibodies to capture and detect human thyroglobulin. Such polyclonal antibodies likely bind both linear and conformational epitopes along the length of the thyroglobulin molecule.

A more stringent test to evaluate the nature of soy-derived thyroglobulin would be the use of a second ELISA procedure which utilizes monoclonal antibodies for capture and detection, respectively. The commercially available ELISA produced by Kronus, Inc. (Boise, Id.) is such an assay, and employs monoclonal antibodies which can simultaneously recognize two different conformational determinants on human thyroglobulin. This assay was used to detect the presence of thyroglobulin in selected soy protein samples that were identified as expressing this protein.

The Organtek kit utilizes two polyclonal antibodies while the Kronus kit utilizes two monoclonal antibodies for detection. A third sandwich-based ELISA was developed and this ELISA utilized a monoclonal antibody for capture and a polyclonal antibody for detection. Briefly, 500 ng of capture antibody (GTX21984, GeneTex, Irvine, Calif.) was coated onto ELISA plates by incubation at 4° C. for 16 hours. Unbound antibody was washed with PBS and nonspecific binding sites were blocked by incubation with 1% BSA in PBS for 1 hour at 23° C. Soy protein samples and the hTG standard were then loaded onto plates and allowed to complex with the bound antibody for 2 hours at 23° C. Unbound products were washed and a rabbit polyclonal detection antibody (GTX73492, GeneTex, Irvine, Calif.) was allowed to bind to the antigen for 2 hours at 23° C. The secondary antibody was subsequently detected using a goat anti-rabbit IgG-HRP antibody (sc2004. Santa Cruz Biotechnology, Santa Cruz, Calif.) by incubation for 1 hour at 23° C. The antibody-antigen complexes were incubated with TMB Substrate (BioFX, Owings Mills, Md.), and colorimetric reactions were stopped by the addition of 0.6 M sulfuric acid. Absorbance values were read at 450 nm and confirmed the results of the two commercial assays. The fact that separate monoclonal antibodies reacted with the soy-derived transgenic protein, along with the fact that two separate commercial kits detected seed-specific immunoreactive proteins, provided further support for the authenticity of recombinant hTG protein.

### Sephacryl S-300 HR Gel Filtration Chromatography of Soybean-Derived Thyroglobulin

To begin a physico-chemical characterization of soybean-derived thyroglobulin, gel filtration chromatography (size exclusion chromatography) was used on total soluble protein isolated from ELISA-positive seeds. A Sephacryl S-300 HR gel filtration column (bed height 72 cm) was calibrated with molecular weight standards by monitoring absorbance at 280 nm (BioLogic LP, BIO-RAD, Inc.).

Next, total soluble protein isolated from ELISA-positive seeds was then applied to this gel filtration column. Protein elution was monitored and individual fractions of separated protein were collected.

Similarly, human thyroid-purified thyroglobulin (Calbiochem. Inc.) protein was diluted in 0.5 ml of wild type soy protein, and applied to the same column. Eluted fractions were also collected.

Eluted fractions were then subjected to ELISA (Orgentec) to detect the presence of immunoreactive thyroglobulin in each fraction. Immunoreactive profiles for human thyroid-purified thyroglobulin and soybean-derived thyroglobulin were similar by comparison. Thyroglobulin is approximately 330 kDa as a monomer, but exists in solutions as a 660 kDa dimer. Therefore it was of interest to determine whether soybean-derived thyroglobulin could also form dimers. Both thyroglobulin protein preparations had a peak elution volume similar to that observed for bovine thyroglobulin (at 669 kDa). In fact, it appears that soybean-derived thyroglobulin was somewhat more homogenous in its elution profile than that observed for human thyroid-purified thyroglobulin since the peak was sharper. More importantly, it was clear from these studies that soybean-derived thyroglobulin could form ˜660 kDa dimers, strongly suggesting that this protein folds in a manner similar to thyroid-isolated human thyroglobulin, allowing dimer formation.

### Gel Filtration Chromatography and Western Blot Analysis of Soybean-Derived Thyroglobulin and Thyroid Purified Thyroglobulin and Quantification of Recombinant Protein in Seed Extracts:

In another embodiment, a sephacryl S-300 HR gel filtration column (bed height 72 cm) was calibrated by determining the peak elution volumes (absorbance at 254 nm, BioLogic LP, BIO-RAD. Inc.) of a set of molecular weight protein standards (Sigma. Inc.). Crude, total soluble protein was then isolated from hTG-positive seeds, and applied to a gel filtration column, and eluted fractions were collected. Similarly, human thyroid-purified thyroglobulin was applied to the same column, and eluted fractions were also collected. Eluted fractions were then subjected to ELISA (Orgentec) to detect the presence of immunoreactive thyroglobulin in each fraction.

Based on gel filtration chromatography it was clear that soybean-derived thyroglobulin could form 660 kDa dimers. This result suggested that monomers would have a size of approximately 330 kDa. To prove this possibility, protein extracts from transgenic and wild type seeds were run in 5% native polyacrylamide gels for approximately 2 hours at 110V. Unless noted, neither the gel, sample buffer nor running buffer contained β-mercaptoethanol or SDS, and samples were not boiled prior to loading onto the gel. Purified hTG (EMD Chemicals, Gibbstown, N.J.) was included as a standard. Following electrophoresis, gels were equilibrated in 1× N-cyclohexyl-3-aminopropanesulfonic acid buffer at (pH 11) with 10% methanol for 10 minutes and transferred to Immobilon-P membrane (Millipore, Billerica, Mass.). Membranes were blocked overnight with 5% nonfat milk in PBS solution at 4° C., incubated with rabbit anti-hTG polyclonal antibody (Gene Tex Inc., Irvine, Calif.) for 3 hours at 23° C., and washed three times (10 minutes each) with PBS containing 0.05% Tween. Membranes were then incubated with goat anti-rabbit HRP (horse radish peroxidase)-conjugated IgG (Santa Cruz Biotechnology, Santa Cruz, Calif.) for 30 minutes at 23 odegree. C. and washed. Detection was carried out using the SuperSignal West Pico substrate (Thermo Scientific, Rockford, Ill.).

Alternatively, gel filtration chromatography was used to partially purify proteins from crude soluble seed extracts. A Sephacryl S-300 HR gel filtration column was calibrated by determining the peak elation volumes of a commercial set of molecular mass standards ranging in size from 669 kDa to 29 kDa. The largest of these molecular mass standards was bovine thyroglobulin (MW ˜669 kDa) and eluted in fraction 20. β-amylase was the standard migrating at 443 kDa and alcohol dehydrogenase is the standard at 200 kDa. Following calibration, transgenic seed extract from line 77-5 was applied to the Sephacryl column, and the eluted protein in each fraction was subjected to an ELISA for detection of hTG. The immunoreactive profile for soy-derived hTG showed that fractions 17-23 contained detectable levels of hTG, with peak immunoreactivity localized to fractions 20 and 21. Fractions 1-11 and 28-36 showed minimal absorbance. The elution profile for soy-derived hTG was consistent with the elution of the bovine thyroglobulin standard in fraction 20, suggesting that seed-specific hTG is likely folded and charged in a manner similar to that of the bovine thyroglobulin marker. For comparison, commercially purified hTG was also chromatographed on a Sephacryl column and fractions were similarly assayed for immunoreactivity. The elution profile of commercially-purified hTG suggests that this protein is more heterogeneous than soy-derived hTG since high levels of immunoreactivity were detected in a broad peak throughout fractions 18-22. These results also suggest that purified hTG is slightly heavier than soy-derived hTG, consistent with the likely iodination of the human sample but not the soy-derived sample.

Western analysis was performed to visualize immunoreactive protein in the eluted fractions. Equivalent volumes of partially-purified seed protein and commercially-purified hTG were separated in native polyacrylamide gels and subjected to western analysis. Equal amounts of protein from the indicated fractions were separated in 5% native gels and subjected to western analysis. As expected, the migration of soy hTG in extracts following partial purification was analogous to that of the commercially purified hTG, further demonstrating the molecular similarities of both proteins when characterized under a variety of sizing and separating conditions.

### Confocal Microscopy

Confocal microscopy to visualize subcellular localization within seed cotyledon tissue was performed as follows. Whole seed tissue was imbibed for 16 hours in 1×PBS and the seed coat was removed. Tissue was fixed as described previously by our laboratory (XX, Piller 2005, Oakes 2008, Powell 2011). Briefly, sections were permeabilized with 1×PBS containing 0.2% Tween for 10 minutes, and nonspecific binding was blocked by incubation with 1×PBS supplemented with 3% BSA for 4 hours at 23° C. Tissue was incubated with rabbit anti-hTG serum (1:20 dilution) for 16 hours at 4° C. followed by incubation with an AlexaFluor 594 goat anti-rabbit IgG-HRP conjugated secondary antibody (1:200 dilution) for 1 hour at 23° C. Finally, tissue was incubated with 4,6-diamidino-2-phenylindole (DAPI; 1:500 dilution) for 5 minutes. Cover slips were added to the sections using Gel/Mount aqueous mounting media. Images were collected with a LSM 710 Spectral Confocor 3 Confocal Microscope (Carl Zeiss, Inc.) using a 40× objective and a 405 nm laser to visualize DAPI stained nuclei, along with a 561 nm laser to collect emitted fluorescence from the Alexafluor 594 antibody. Stacks of images (30 optical sections, 17 nm apart) were collected in the Z plane of the specimens and projected to form a single image. To improve clarity and reproduction quality, image colors were proportionally enhanced using the ZEN 2009 Light Edition software. In the observed confocal imagery, heterologous protein will fluoresce as a red/orange color while the DAPI stained nucleic acid will fluoresce as blue color. In the case of hTg, the AlexaFluor antibody detected protein strongly associated with the intracellular membrane.

For western visualization and quantification, known amounts of commercially-purified hTG protein and crude seed-extracted protein (line 77-5) were incubated with SDS-sample buffer lacking β-mercaptoethanol, and electrophoresed in 5% native polyacrylamide gels. Western blots were performed and X-ray films of the resulting blots were scanned for densitometric analysis. Integrated density was measured using ImageJ software. The image was inverted and background pixel values were subtracted. A standard curve was plotted using these integrated density values and the known amounts of purified hTG protein, from which an absolute value of hTG in the seed sample was determined. For ELISA quantification, known amounts of hTG (0.01 ng-10 ng) and crude seed extracted protein (10-fold dilutions over four orders of magnitude) were coated onto ELISA plates and processed as described above. Absorbance values from the known concentrations of hTG were used to generate a curve, and the concentrations of hTG in seed extracts was determined by extrapolation of hTG concentration for those samples with absorbance values falling within the linear range of the curve. Absolute values were converted to a percentage of total protein.

### Purification of Soy-Derived hTg Protein

Soy-derived hTg can be purified from soybean seed proteins using traditional biochemical methods such as ion-exchange chromatography, ammonium sulfate precipitation and size exclusion chromatography, etc. Transgenic soybean seeds expressing recombinant human thyroglobulin were ground to a fine powder in a coffee mill, and seed protein was extracted with 0.5×PBS buffer using sonication as described above. The pH of the soluble protein solution was adjusted to pH 5.8 with acetic acid which resulted in the precipitation of protein classes. The solution was clarified by centrifugation at 16,100×g for 5 minutes at 4° C. Ammonium sulfate powder (AS) was added to a final concentration of 40% saturation to precipitate unwanted proteins. Precipitated proteins were collected by centrifugation and discarded. The concentration of AS was then increased to 45% of saturation to precipitate the hTg protein. The soy hTG and other precipitated proteins were collected by centrifugation as described above, and the proteins were then suspended in a buffer containing 50 mM Tris-Cl (pH 7.5) and transferred to dialysis tubing (Fisher Scientific) with a molecular weight cutoff of 12,000-14,000 daltons. The suspended proteins were dialyzed for 16 hours at 4° C. in binding buffer (50 mM Tris pH 7.5) and then mixed with DEAE cellulose (Sigma). The protein and DEAE mixture was rocked gently for 1 hour at 4° C. and then the beads were pelleted by centrifugation at 3,000×g for 5 minutes. The DEAE resin was washed extensively and then transferred to a small separation column (Bio-Rad). Bound proteins were eluted using a NaCl step gradient. Proteins eluting in the 100 mM and 150 mM NaCl step fractions were concentrated in 0.5×PBS and loaded onto a sizing column containing Sephacryl 300 resin. Fractions containing purified thyroglobulin were pooled, concentrated, and quantified using the Bradford reagent and the in-house ELISA described above. Protein from each purification step was separated by PAGE using 3-20% native and 4-15% denaturing gradient gels. Gels containing separated protein were visualized with Coomassie blue stain.

FIG. 21 shows acrylamide gels and western blots of protein samples collected from each step of the purification process. The amount of soluble protein in each collected sample was determined using the Bradford assay with BSA as a protein standard. The “start” sample contained 40 ug of extracted soy protein; the “pH drop” sample contained 20 ug of soluble protein following a pH drop pH5.8; the “45% AS cut” sample contained 10 ug of protein that precipitated out of solution in the 40-45% ammonium sulfate range; the “DEAE” sample contained 4 ug of protein that eluted from DEAE resin following 100 mM and 150 mM NaCl step elution gradients; and the “S300“sample contained 1 ug of protein from peak protein fractions separated by Sephacryl 300 resin. For comparison of soy-htg with commercial hTg, 1 ug of commercial hTg protein (Calbiochem) was loaded in the lane next to lane containing purified soy-hTg (S300 lane) on the denaturing gel.

Protein samples were electrophoresed for approximately 1.5 hours at 110V. Neither the gel nor the running buffer contained β-mercaptoethanol, and only denaturing conditions involved SDS in sample buffer, gels and running buffers. Samples were not boiled prior to loading onto gels.

For western blot analysis, the electrophoresed gels were equilibrated in 1×CAPS (pH 11) buffer with 10% methanol for 10 minutes and transferred to Immobilon-P membrane (Millipore, Billerica, Mass.). Membranes were blocked overnight with 5% nonfat milk in PBS solution at 4° C., incubated with rabbit anti-hTG polyclonal antibody (Gene Tex Inc., Irvine, Calif.) for 3 hours at 23° C., and washed three times (10 minutes each) with PBS containing 0.05% Tween. Membranes were then incubated with goat anti-rabbit HRP-conjugated IgG (Santa Cruz Biotechnology, Santa Cruz, Calif.) for 30 minutes at 23° C. and washed as described above. Detection was carried out using the SuperSignal West Pico substrate (Thermo Scientific, Rockford, Ill.).

FIG. 21 shows that soy-hTg was effectively purified from soybean seed proteins using the biochemical methods outlined above. The samples loaded onto the native gel clearly shows the purification of two protein bands which represent the dimeric and monomeric forms of hTg. The dimeric form (upper arrow on native gel) is more abundant than the monomeric form, and demonstrates that the multimeric protein remained intact during the various purification steps. On the denaturing gels, only a single band was detected, representing the monomeric form of hTG. It should be noted that monomeric forms of protein can be visualized in all the samples, including faint amounts visible in the starting material. The presence of heterologous protein in total, unpurified seed protein indicates the abundance of the hTg protein that accumulated in seeds.

The western blot of the denaturing gel also shows soy-hTg protein in all samples. It is of interest to note that the purified soy-hTg protein migrates as a single “tight” band on this gradient gel while the commercially-purified human thyroid-derived sample runs as a “smear” with several protein species detected with faster and slower mobilities than the bulk hTg. This result shows some of the issues of heterogeneity and nonuniformity that are associated with hTg protein purified from human tissue. The soy-purified hTg is a much more uniform sample. It migrates slightly faster than commercial hTg, presumable because it does not contain iodine residues which are present in the human protein.

Approximately 20% of thyroid cancer patients develop anti-thyroglobulin antibodies. These autoantibodies can bind thyroglobulin and interfere with current FDA-approved thyroglobulin immunoassays. In additional studies, the inventors made use of some patients' sera to demonstrate the ability of these autoantibodies to bind soybean-derived thyroglobulin.

For these studies, thyroid-isolated thyroglobulin (Calbiochem, Inc.) or soybean-derived thyroglobulin were separately fractionated on a Sephacryl S-300 HR gel filtration column. Following gel filtration, fractions representing 59 to 60 milliliters of column void volume for thyroid-isolated and soybean-derived thyroglobulin were concentrated (using a Centricon-100). Quantification of the concentrated protein was accomplished using Bradford assays. Equivalent amounts (100 ng/well) of each thyroglobulin preparation were coated onto ELISA microtiter plates (Nunc high-binding) overnight as is routine in the inventor's laboratory. After blocking and washing, a 1:50 dilution of selected patients' sera and control sera were incubated on each coated plate. Two hours later, a peroxidase-conjugated anti-human IgG antibody was added. Bound anti-thyroglobulin autoantibodies were detected by the addition of substrate, and determining absorbance at 450 nm.

Regardless of the source of thyroglobulin used to coat plates, there was no significant difference in the ability of autoantibodies in patients' sera to recognize soybean-derived or thyroid-isolated thyroglobulin. These results further demonstrate the antigenic identity of these two thyroglobulin isolates and suggest that the soybean derived thyroglobulin is similar to if not identical to at least one conformer of human wild type thyroglobulin.

Six to eight week old female Balbic mice were gavaged every other day for 26 days as follows: using a 22 gauge feeding needle, 200 ul of soymilk protein extract from either wild type (non-transformed) seeds or transgenic seeds expressing hTG was administered to each animal via oral gavage. On day 14, both groups were immunized intraperitoneally with 100 ug of commercial human thyroglobulin (Calbiochem, UK) in aluminum hydroxide gel as an adjuvant (Sigma-Aldrich, St Louis, Mo.).

Following euthanasia on day 42, sera was collected for ELISA analyses. ELISA plates were coated with 100 ng of commercial hTG (Calbiochem) overnight at 4° C. Plates were then washed with PBS and blocked with 1% BSA-PBS for 1 hour. After a second wash, 100 ul of sera samples of varying dilutions were loaded on to the plate and incubated at room temperature for 2 hours. Following a third PBS wash, 100 ul of anti-mouse IgG-HRP antibody (Southern Biotech) at 1:500 dilution was added to each well and allowed to incubate for 1 hour. The antibody-antigen complexes were coated with TMB Substrate (BioFX, Owings Mills, Md.), and colorimetric reactions were stopped by the addition of 0.6 M sulfuric acid. Absorbance values were read at 450 nm.

At three different dilutions there is a difference in antibody titers between the mice receiving wild type soymilk (WT) and the mice receiving soy-derived Tg (hTG). This suggests the mice that received the hTG soymilk formulation induced, at least partially, either a high or low-dose tolerance response to the antigen in the milk.

Dilutions of mouse sera from wild type (WT) and thyroglobulin (hTG) groups were analyzed. Eight serial dilutions of each sample were tested in the ELISA and absorbance values determined.

In addition, splenocytes were isolated for T-cell restimulation assays. Spleens were ground through 30 mesh screens to isolate leukocytes. Resulting cells were cultured in RPMI-1640 with 20% FBS (BD Biosciences, Chicago, Ill.). Cells were plated at 106 cells per well in 96-well flat bottom tissue culture plates, coated with 10 ug commercial hTG or FBS and incubated for 72 hours. The supernatants from these cell cultures were analyzed for IFN-γ and 11-4 production via ELISA. The decreased production of IFN-γ indicates a shift to an anergic response by the T-cells to the stimulus. This is further supported by the high doses of tolerogen (280 ug) administered in each gavage.

Splenocytes from wild type (WT) and thyroglobulin (hTG) groups were restimulated using commercial thyroglobulin (TG) and Fetal Bovine Serum (FBS) as a control. Supernatants were collected and analyzed via ELISA for the presence of IFN-γ. One way analysis of variance (ANOVA) indicated a statistically significant difference between IFN-γ production in wild type splenocytes as compared to thyroglobulin group splenocytes (p=01.01).

Thus, this example shows heterologous production of a large, complex, difficult-to-express, glycosylated protein in soybean seeds. To date, the expression of hTg in soybeans represents the largest recombinant protein to be expressed in any plant host system. The recombinant hTg protein produced in soy was shown to be functional by a variety of assays when compared with commercial hTg purified from human thyroids. The recombinant protein was easily purified in several basic biochemical steps from other soybean seed proteins showing the ease with which heterologous proteins can be purified from other soybean seed proteins. This was due in part to the abundance of hTG expressed in seeds (e.g. >1% TSP) and the relatively low complexity of endogenous seed proteins. The expression of hTg was made possible by synthetic gene design which included optimization of codons for expression in soybean, and removal of unfavorable destabilizing sequences. A signal peptide was included in the gene design to target protein translation to the secretory pathway and allow accumulation at an optimal subcellular location within the cell. Similar strategies would be employed to express proteins involved with the deconstruction of lignin, hemicellulose and cellulose in soybean seeds and enumerated in Tables 1, 2 and 3.

### Example for Designing Genes, Soybean Transformation, Characterization, Powder Formulation, and Storage.

Using the above example for thyroglobulin and the procedures enumerated therewith, the enzymes of tables 1, 2, and 3 will be used in identical or similar procedures to generate synthetic genes optimized for stable expression in soybean seeds. As explained above with reference to thyroglobulin, the AA sequences of the various enzymes that appear in tables 1, 2, and 3 will be analyzed for the presence of a signal peptide. If a native signal peptide is present it will be incorporated into the gene design. If a native signal peptide (SP) is not present, one will be chosen from those previously known to function in soybean (e.g. signal peptides derived from soybean glycinin, Arabidopsis chitinase and S. aureus SEB). Synthetic gene variants can be made that utilize different SPs to test heterologous protein stability of enzymes at various subcellular locations within the seed. For example, one variant of the enzymes in Tables 1, 2 and 3 may contain a native signal peptide for internal localization while a second variant may contain the SEB signal peptide for extracellular localization. These two variants could be tested alongside a third variant that lacks a signal peptide for cytosolic localization. Synthetic genes and any variants encoding enzymes listed in Tables 1, 2 and 3 will be engineered with 5′ NcoI and 3′ XbaI restriction sites to facilitate cloning into pTN200 binary vector. The resulting binary vectors will be transformed into Agrobacterium strains compatible with soybean transformation methods described previously for hTG (Powell 2011) and FanC (Piller 2005).

Progeny from transformed lines (e.g. T1 seeds) will be screened by methods similar to and/or identical to those described for thyroglobulin, to identify specific transgenic events with optimal heterologous enzyme accumulation. Those optimal events will then be propagated and characterized over multiple generations. Typical methods for gene and protein characterization include foliar sprays to confirm herbicide tolerance of plants, PCR to verify the presence of the transgene, northern blots to verify the presence of heterologous mRNA species, protein assays to quantify heterologous protein, western blots and ELISAs to verify the presence of the heterologous protein and for protein quantification, Southern blots to determine complexity of the inserted T-DNA (e.g. gene copy number, loci number), confocal microscopy and immunohistochemistry to visualize subcellular localization, and specific enzyme assays to evaluate enzyme activity and substrate specificity.

Transgenic seeds expressing heterologous enzymes from Tables 1, 2 and 3 can be ground to a specified particle size, or alternatively ground to a fine powder with subsequent sieving or screening to identify different particle size classes. The removal of the hull (seed coat) will increase overall protein levels within a given volume of powder biomass since seed hulls represent ˜10% of soybean seed biomass yet contain little protein. There are also known methods for removing oils and/or carbohydrates from seeds and seed powder products as oils and carbohydrates also comprise a significant amount of seed biomass. One such method includes hexane extraction of oil and ethanol extraction of carbohydrate.

Transgenic soybeans and ground powder compositions expressing enzymes in Tables 1, 2 and 3 can be stored as described above and as previously described for FanC (Piller 2005). Under those storage conditions (ambient laboratory conditions of ˜22° C. and ˜50% relative humidity) heterologous FanC protein was shown to remain stable for 8 years with no detectable degradation in whole seeds and ground powder. The transgenic seeds and ground seed powder can also be stored in vacuum-sealed containers, and at temperatures and RH lower than those considered to be “ambient” for a laboratory setting (e.g. temperature lower than 22° C. and RH lower than 50%).

Once powders are made from each transgenic soy line expressing a particular enzyme from Tables 1, 2 and 3, seed powder compositions representing unique enzyme cocktails will be formulated. Such compositions could contain seed powder from one or many different enzymes mixed together in desired ratios. As an example, a cocktail containing powder made from a transgenic soy line expressing the CelA from Caldocellum saccharolyticum could be combined with powder made from a transgenic soy line expressing the CelO from Clustridium thermocellum, and combined with the powder made from a transgenic soy line expressing the b-glucosidase from Volariella volacea. Each powder could be combined in varying percentages, representing the specific activities of each enzyme expressed by that particular soy line which would provide efficient glucose generation from any particular source of lignocellulose biomass. For example, the deconstruction of corn stover might include ratios of 4:1:1 volume to volume of CelA to CelO to b-glucosidase powder, depending on the specific activities per gram of each powder and the dissolution rates. Compositions could also contain particles of various sizes to allow staggered or differential release of heterologous enzymes. Any custom seed powder compositions could also be stored for long periods of time as described for FanC (Piller 2005).

In an embodiment, the present invention relates to a composition comprising a transgenic soy plant that has been transformed with one or more genes that expresses one or more enzymes, said one or more enzymes being capable of at least partially metabolizing and/or deconstructing cellulose, lignin, and/or hemicellulose. In a variation, the one or more enzymes are expressed in soybean seeds.

In a variation, the enzyme is one or more members comprising laccases, peroxidases, oxidases, xylanases, xylosidases, endoglucanases, glucosidases, mannanases, mannosidases, or cellulases. Throughout the specification, when these above enzymes are cited, it is contemplated and therefore within the scope of the invention that the enzyme commission (EC) number (i.e., the numerical classification scheme for enzymes based upon the chemical reaction that they perform) be used to include all enzymes in the class that fall within the EC number (even if the enzyme is given a different name). The EC numbering system is explained in Enzyme Nomenclature 1992 [Academic Press, San Diego, Calif., ISBN 0-12-227164-5 (hardback), 0-12-227165-3 (paperback)] with Supplement 1 (1993), Supplement 2 (1994), Supplement 3 (1995), Supplement 4 (1997) and Supplement 5 (in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650; respectively), all of which are incorporated by reference in their entireties.

The enzymes in Tables 1, 2, and 3 have the following EC numbers associated with them: laccases (EC 1.10.3.2), peroxidases (EC 1.11.1.14 or EC 1.11.1.13 or EC 1.11.1.16), oxidases (EC 1.1.3.13), xylanases (EC 3.2.1.8), xylosidases (EC 3.2.1.37), endoglucanases (EC 3.2.1.151), glucosidases (EC 3.2.1.21 or EC 3.2.1.74), mannanases (EC 3.2.1.78), mannosidases (EC 3.2.1.25), and cellulases (3.2.1.4 or 3.2.1.91).

In a variation, a genus is contemplated and therefore within the scope of the invention for compositions, powder, other products as well as methods that has only the particular enzymes enumerated in Tables 1, 2, and 3 or any subgenus therein.

In an embodiment, the present invention relates to a composition that comprises at least a first enzyme and a second enzyme, said first enzyme and said second enzyme both being capable of metabolizing cellulose, lignin, and/or hemicellulose, wherein either of said first enzyme or said second enzyme is present at a concentration of at least 2 g/800 g of soy. Optionally, the composition is in powder form. It is contemplated and therefore within the scope of the invention that the yield of enzyme (or the one or more enzymes) without further purification in the transgenic plant will be more than about 1 g/800 g of soy, or alternatively, more than about 1.5 g/800 g of soy, or alternatively, more than about 3 g/800 g of soy, or alternatively, more than about 4 g/800 g of soy, or alternatively, more than about 5 g/800 g of soy, or alternatively, more than about 7.5 g/800 g of soy, or alternatively, more than about 10 g/800 g of soy.

In an embodiment, the present invention relates to a transgenic soy plant that has been transformed with one or more genes that expresses one or more enzymes, said one or more enzymes being capable of metabolizing and/or deconstructing cellulose, lignin, and/or hemicellulose.

In an embodiment, the present invention relates to a powder derived from soy seed, wherein said powder comprises a transgenic soy plant that has been transformed with one or more genes that expresses one or more enzymes, said one or more enzymes being capable of at least partially metabolizing cellulose, lignin, and/or hemicellulose, said powder being of a size that is smaller than 1600 micron in size, or alternatively, smaller than about 500 micron in size, or alternatively, smaller than about 200 micron in size.

In an embodiment, the present invention relates to a powder derived from soy seed, wherein said powder comprises a transgenic soy plant that has been transformed with a gene that expresses an enzyme, said enzyme being capable of at least partially metabolizing and/or deconstructing cellulose, lignin, and/or hemicellulose. In an embodiment, said powder is of a size that is smaller than 1600 micron in size, or alternatively, smaller than about 200 micron in size.

In an embodiment, the present invention relates to a soy seed comprising at least one overexpressed enzyme that is capable of at least partially metabolizing cellulose, lignin, and/or hemicellulose.

In an embodiment, the present invention relates to a powder made from a transgenic soy plant, said powder comprising at least one enzyme that is capable of at least partially metabolizing cellulose, lignin, and/or hemicellulose, said powder made by transforming a soy plant with a gene that expresses said at least one enzyme, expressing said at least one enzyme in said soy plant to generate a soy plant with an expressed at least one enzyme, micronizing said soy plant with said expressed at least one enzyme wherein said powder containing said at least one enzyme is in a form that allows said at least one enzyme to remain functional at a level that is at least 90% relative to freshly prepared enzyme after a period of at least 12 months. In one variation, the enzyme remains functional at a level that is at least 90% relative to freshly prepared enzyme for a period of 18 months, or 24 months, or 36 months, or 48 months, or alternatively, 5 years.

In an embodiment, the enzyme can be shipped and/or stored in the absence of a cold chain. In a variation, this allows the enzyme to shipped and/or stored for a period of 18 months, or 24 months, or 36 months, or 48 months, or alternatively, 5 years.

In an embodiment, the production costs are below industry standards for recombinant manufacturing. In a variation, the production costs are sufficiently low to allow profitable applications.

In an embodiment, the present invention relates to a method of making a product (e.g., a powder) that contains at least one enzyme that is capable of at least partially being able to metabolize cellulose. lignin, and/or hemicellulose, said at least one enzyme derived from a transgenic soy plant that contains a gene that expresses said at least one enzyme, expressing said at least one enzyme (optionally using a promoter), and optionally micronizing the soy plant containing the expressed at least one enzyme, wherein the product (or powder) is of a size that is no larger than 150 microns.

In an embodiment, the present invention relates to a powder that comprises a transgenic soy plant; said transgenic soy plant comprising at least one enzyme derived from at least one gene that expresses said at least one enzyme, said at least one enzyme capable of at least partially metabolizing cellulose, lignin, and/or hemicellulose, said powder having said at least one enzyme present at a concentration of 2 g enzyme/800 g powder, said powder being of a size that is no larger than 1600 microns, said at least one enzyme being capable of at least partially being able to metabolize cellulose, lignin, and/or hemicellulose for a period of no less than 6 months, said powder being in a form that allows said powder to be combined with a second powder that comprises at least a second enzyme that is capable of at least partially metabolizing cellulose, lignin, and/or hemicellulose.

In an embodiment, the present invention relates to a method of at least partially metabolizing cellulose, lignin, and/or hemicellulose, comprising treating said cellulose, lignin, and/or hemicellulose with a powder, wherein said powder is derived from a transgenic soy plant, said transgenic soy plant being transformed with a gene that expresses at least one enzyme that is capable of at least partially metabolizing cellulose, lignin, and/or hemicellulose.

In an embodiment, the present invention relates to a composition comprising a transgenic soy plant that has been transformed with a gene that expresses an enzyme, said enzyme being capable of at least partially metabolizing lignin, hemicellulose, and/or cellulose wherein the enzyme is present at a concentration of at least 2 g/800 g of soy powder without additional concentration, filtration, or lyophilization. In a variation, the concentration may be at least 1, 2, or 4 g/800 g of soy powder without additional concentration, filtration, or lyophilization. The enzyme may be present at a concentration of at least 4 g/800 g of soy powder.

In an embodiment, the powders can be homogenized, allowing intra-lot consistency, such that there is a variance of less than about 10% from one lot to the next (when analyzed by random samples).

In one embodiment, the composition may comprise an enzyme that is one or more of laccases, peroxidases, xylanases, endoglucanases, cellulases, or glucosidases.

In one embodiment, the composition is in a powder form, wherein 90% of the powder has a particle size of about 5 to 6000 microns. In a variation, the composition is in a powder form, wherein 90% of the powder has a particle size of about 280 to 4000 microns. In a variation, the composition is in a powder form, wherein 90% of the powder has a particle size of about 280-1600 microns. In a variation, the composition is in a powder form, wherein 90% of the powder has a particle size of about 520-860 microns, or alternatively, 520-1600 microns, or alternatively 860-1600. In a variation, 90% of the powder has a particle size less than about 1600 microns, or alternatively, less than about 500 microns, or alternatively, less than about 200 microns in size.

In an embodiment, the present invention relates to a composition that comprises at least a first enzyme and a second enzyme, said first enzyme and said second enzyme being capable of at least partially metabolizing lignin, hemicellulose, and/or cellulose. In a variation, the first enzyme and the second enzyme are one or more members selected from the group consisting of laccases, peroxidases, xylanases, endoglucanases, cellulases, and glucosidases. In a variation, the composition is in a powder form, wherein 90% of the powder has a particle size of about 5 to 1600 microns.

In an embodiment, the present invention relates to a powder derived from soy seed, wherein said powder comprises a transgenic soy plant that has been transformed with a gene that expresses an enzyme, said enzyme being capable of at least partially metabolizing cellulose, lignin, and/or hemicellulose, said powder being of a size between about 5-1600 micrometers to facilitate dissolution. In a variation, said enzyme is one or more members selected from the group consisting of laccases, peroxidases, xylanases, endoglucanases, cellulases, and glucosidases. In a variation, the powder containing the enzyme has an enzyme that retains at least 80% activity relative to freshly expressed enzyme after about one year at room temperature.

In an embodiment, the present invention relates to a powder made from a transgenic soy plant, said powder comprising an enzyme that is capable of at least partially metabolizing cellulose, lignin, and/or hemicellulose, said powder made by transforming a soy plant with a gene that expresses said enzyme, expressing said enzyme in said soy plant to generate a soy plant with an expressed enzyme, micronizing said soy plant with said expressed enzyme until it is a size that is about 5-1600 micrometers, wherein said powder containing said enzyme is present at a concentration of at least 2 g enzyme/800 g of soy powder without additional concentration, filtration, or lyophilization, and wherein said powder is in a form that allows said enzyme to remain functional at room temperature for a period of at least 12 months with less than 20% loss of enzymatic activity.

In a variation, the powder is derived from one or more soy seeds. In an embodiment, the enzyme is present at a concentration of at least 4 g enzyme/800 g of soy powder without additional concentration, filtration, or lyophilization. In one variation, the enzyme is present at a concentration of at least 6 g enzyme/800 g of soy powder without additional concentration, filtration, or lyophilization.

In an embodiment, the present invention relates to a transgenic soy product that is in powder or flake form, said soy product comprising an overexpressed enzyme, said soy product being comprised of at least a first harvest and a second harvest wherein a variance between enzyme activity from the first harvest and the second harvest is less than about 10%. In a variation, the enzyme is one or more members selected from the group consisting of laccases, peroxidases, xylanases, endoglucanases, cellulases, and glucosidases.

In one embodiment, the present invention is related to a method of making ethanol. The method of making ethanol may comprise adding any of the compositions listed above to a plant or a partially metabolized plant.

In one embodiment, the present invention relates to a method of at least partially metabolizing and/or deconstructing cellulose, lignin, and/or hemicellulose. In a variation, the method comprises treating said cellulose, lignin, and/or hemicellulose with a powder or enzyme, wherein said powder is derived from a transgenic soy plant, said transgenic soy plant being transformed with a gene that expresses an enzyme that is capable of at least partially metabolizing cellulose, lignin, and/or hemicellulose. In a variation, the enzyme is one or more members selected from the group consisting of laccases, peroxidases, xylanases, endoglucanases, cellulases, and glucosidases. In an alternate variation, the powder is of a size that has 90% in a range between about 5 and 1600 microns.

In a variation, the powders may be derived from transgenic soy bean seeds, wherein the transgenic soy bean seeds may be transformed with a gene that expresses an enzyme that is capable of at least partially metabolizing and/or deconstructing cellulose, lignin, and/or hemicellulose. In a variation, the powders in the method may contain one or more enzymes that can be combined in varying percentages to achieve a cocktail of enzymes. The size of this powder may be less than about 1600 microns, or alternatively, less than about 500 microns, or alternatively, less than about 200 microns in size (or alternatively, between the sizes of the ranges listed above). In a variation, the powders containing one or more enzymes may be added sequentially to reaction mixtures over time. In a variation, the powders containing one or more enzymes may be dissolved in aqueous solutions and combined in varying percentages to achieve a cocktail of enzymes prior to being added sequentially to reaction mixtures over time.

It should be understood that it is contemplated and therefore within the scope of the present invention that any one or more feature that is disclosed herein can be combined with any other one or more feature that is disclosed herein even if those features are not discussed together. Moreover, although features may be discussed together, it should be understood that those features do not necessarily have to go together. That is, it is contemplated and therefore within the scope of the invention that those features may be separated. When ranges are mentioned, any integral number that falls within that range is contemplated as an endpoint (for example, if a range of 1-10 is mentioned, it is contemplated, that endpoints may include 2, 3, 4, 5, 6, 7, 8, or 9). If a genus is enumerated, it should be understood that all subgenera that fit within the scope of that genus are contemplated as features of the invention. Moreover, minor modifications can be made to the invention without departing from the spirit and scope of the present invention. Nevertheless, the below claims define the invention.

The following references are incorporated by reference in their entireties.
Acharya, S. and A. Chaudhary (2012). “Bioprospecting thermophiles for cellulase production: a review.” Braz J Microbiol 43(3): 844-856.
Asada, Y., A. Watanabe, T. Irie, T. Nakayama and M. Kuwahara (1995). “Structures of genomic and complementary DNAs coding for Pleurotus ostreatus manganese (II) peroxidase.” Biochim Biophys Acta 1251(2): 205-209.
Austin, S., E. T. Bingham, R. G. Koegel, D. E. Mathews, M. N. Shahan, R. J. Straub and R. R. Burgess (1994). “An overview of a feasibility study for the production of industrial enzymes in transgenic alfalfa.” Ann N Y Acad Sci 721: 234-244.
Banerjee, G., J. S. Scott-Craig and J. D. Walton (2010). “Improving Enzymes for Biomass Conversion: A Basic Research Perspective.” Bioenergy Research 3(1): 82-92.
Bauer, M. W., L. E. Driskill, W. Callen, M. A. Snead, E. J. Mathur and R. M. Kelly (1999). “An endoglucanase, EglA, from the hyperthermophilic archaeon Pyrococcus furiosus hydrolyzes beta-1,4 bonds in mixed-linkage (1-->3),(1-->4)-beta-D-glucans and cellulose.” J Bacteriol 181(1): 284-290.
Baunsgaard, L., H. Dalboge, G. Houen, E. M. Rasmussen and K. G. Welinder (1993). “Amino acid sequence of Coprinus macrorhizus peroxidase and cDNA sequence encoding Coprinus cinereus peroxidase. A new family of fungal peroxidases.” Eur J Biochem 213(1): 605-611.
Berka, R. M., I. V. Grigoriev, R. Otillar, A. Salamov, J. Grimwood, I. Reid, N. Ishmael, T. John, C. Darmond, M. C. Moisan, B. Henrissat, P. M. Coutinho, V. Lombard, D. O. Natvig, E. Lindquist, J. Schmutz, S. Lucas, P. Harris, J. Powlowski, A. Bellemare, D. Taylor, G. Butler, R. P. de Vries, I. E. Allijn, J. van den Brink, S. Ushinsky, R. Storms, A. J. Powell, I. T. Paulsen, L. D. Elboume, S. E. Baker, J. Magnuson, S. Laboissiere, A. J. Clutterbuck, D. Martinez, M. Wogulis, A. L. de Leon, M. W. Rey and A. Tsang (2011). “Comparative genomic analysis of the thermophilic biomass-degrading fungi Myceliophthora thermophila and Thielavia terrestris.” Nat Biotechnol 29(10): 922-927.
Berka, R. M., P. Schneider, E. J. Golightly, S. H. Brown, M. Madden, K. M. Brown, T. Halkier, K. Mondorf and F. Xu (1997). “Characterization of the gene encoding an extracellular laccase of Myceliophthora thermophila and analysis of the recombinant enzyme expressed in Aspergillus oryzae.” Appl Environ Microbiol 63(8): 3151-3157.
Berlin, A. (2013). “Microbiology. No barriers to cellulose breakdown.” Science 342(6165): 1454-1456.

Bhalla, A., N. Bansal, S. Kumar, K. M. Bischoff and R. K. Sani (2013). “Improved lignocellulose conversion to biofuels with thermophilic bacteria and thermostable enzymes.” Bioresour Technol 128: 751-759.
Bleve, G., C. Lezzi, S. Spagnolo, G. Tasco, M. Tufariello, R. Casadio, G. Mita, P. Rampino and F. Grieco (2013). “Role of the C-terminus of Pleurotus eryngii Ery4 laccase in determining enzyme structure, catalytic properties and stability.” Protein Eng Des Sel 26(1): 1-13.
Bohlin, C., L. J. Jonsson, R. Roth and W. H. van Zyl (2006). “Heterologous expression of Trametes versicolor laccase in Pichia pastoris and Aspergillus niger.” Appl Biochem Biotechnol 129-132: 195-214.
Bok, J. D., D. A. Yemool and D. E. Eveleigh (1998). “Purification, characterization, and molecular analysis of thermostable cellulases CelA and CelB from Thermotoga neapolitana.” Appl Environ Microbiol 64(12): 4774-4781.
Bost, K. L. and K. J. Piller (2011). Protein expression systems: Why soybean seeds? Soybean: Molecular Aspects of Breeding. A. Sudaric, InTech: 3-18.
Brunecky, R., M. Alahuhta, Q. Xu, B. S. Donohoe, M. F. Crowley, I. A. Kataeva, S. J. Yang, M. G. Resch, M. W. Adams, V. V. Lunin, M. E. Himmel and Y. J. Bomble (2013). “Revealing nature's cellulase diversity: the digestion mechanism of Caldicellulosiruptor bescii CelA.” Science 342(6165): 1513-1516.
Cannella, D. and H. Jorgensen (2014). “Do new cellulolytic enzyme preparations affect the industrial strategies for high solids lignocellulosic ethanol production?“Biotechnol Bioeng 111(1): 59-68.
Chikwamba, R. K., M. P. Scott, L. B. Mejia, H. S. Mason and K. Wang (2003). “Localization of a bacterial protein in starch granules of transgenic maize kernels.” Proceedings of the National Academy of Sciences of the United States of America 100(19): 11127-11132.
Chou, H. L., Z. Dai, C. W. Hsieh and M. S. Ku (2011). “High level expression of Acidothermus cellulolyticus beta-1,4-endoglucanase in transgenic rice enhances the hydrolysis of its straw by cultured cow gastric fluid.” Biotechnol Biofuels 4: 58.
Clemente, T. E., B. J. LaVallee, A. R. Howe, D. Conner-Ward, R. J. Rozman, P. E. Hunter, D. L. Broyles, D. S. Kasten and M. A. Hinchee (2000). “Progeny analysis of glyphosate selected transgenic soybeans derived from Agrobacterium-mediated transformation.” Crop Science 40(3): 797-803.
Clough, R. C., K. Pappu, K. Thompson, K. Beifuss, J. Lane, D. E. Delaney, R. Harkey, C. Drees, J. A. Howard and E. E. Hood (2006). “Manganese peroxidase from the white-rot fungus Phanerochaete chrysosporium is enzymatically active and accumulates to high levels in transgenic maize seed.” Plant Biotechnol J 4(1): 53-62.
Dashtban, M., M. Maki, K. T. Leung, C. Mao and W. Qin (2010). “Cellulase activities in biomass conversion: measurement methods and comparison.” Crit Rev Biotechnol 30(4): 302-309.
Dashtban, M. and W. Qin (2012). “Overexpression of an exotic thermotolerant beta-glucosidase in trichoderma reesei and its significant increase in cellulolytic activity and saccharification of barley straw.” Microb Cell Fact 11: 63.
Dashtban, M., H. Schraft, T. A. Syed and W. Qin (2010). “Fungal biodegradation and enzymatic modification of lignin.” Int J Biochem Mol Biol 1(1): 36-50.
Ding, S., W. Ge and J. A. Buswell (2007). “Molecular cloning and transcriptional expression analysis of an intracellular beta-glucosidase, a family 3 glycosyl hydrolase, from the edible straw mushroom, Volvariella volvacea.” FEMS Microbiol Lett 267(2): 221-229.
Duruksu, G., B. Ozturk, P. Biely, U. Bakir and Z. B. Ogel (2009). “Cloning, expression and characterization of endo-beta-1,4-mannanase from Aspergillus fumigatus in Aspergillus sojae and Pichia pastoris.” Biotechnol Prog 25(1): 271-276.
Eckert, H., B. LaVallee, B. J. Schweiger, A. J. Kinney, E. B. Cahoon and T. Clemente (2006). “Co-expression of the borage Delta(6) desaturase and the Arabidopsis Delta(15) desaturase results in high accumulation of stearidonic acid in the seeds of transgenic soybean.” Planta 224(5): 1050-1057.
Gallardo, O., P. Diaz and F. I. Pastor (2004). “Cloning and characterization of xylanase A from the strain Bacillus sp. BP-7: comparison with alkaline pI-low molecular weight xylanases of family 11.” Curr Microbiol 48(4): 276-279.
Garvey, M., H. Klose, R. Fischer, C. Lambertz and U. Commandeur (2013). “Cellulases for biomass degradation: comparing recombinant cellulase expression platforms.” Trends Biotechnol 31(10): 581-593.
Giardina, P., V. Faraco, C. Pezzella, A. Piscitelli, S. Vanhulle and G. Sannia (2010). “Laccases: a never-ending story.” Cell Mol Life Sci 67(3): 369-385.
Girio, F. M., C. Fonseca, F. Carvalheiro, L. C. Duarte, S. Marques and R. Bogel-Lukasik (2010). “Hemicelluloses for fuel ethanol: A review.” Bioresour Technol 101(13): 4775-4800.
Goodman, D. B., G. M. Church and S. Kosuri (2013). “Causes and Effects of N-Terminal Codon Bias in Bacterial Genes.” Science 342(6157): 475-479.
Goswami, P., S. S. Chinnadayyala, M. Chakraborty, A. K. Kumar and A. Kakoti (2013).
“An overview on alcohol oxidases and their potential applications.” Appl Microbiol Biotechnol 97(10): 4259-4275.
Hahn-Hagerdal, B., M. Galbe, M. F. Gorwa-Grauslund, G. Liden and G. Zacchi (2006). “Bio-ethanol—the fuel of tomorrow from the residues of today.” Trends Biotechnol 24(12): 549-556.
Halldorsdottir, S., E. T. Thorolfsdottir, R. Spilliaert, M. Johansson, S. H. Thorbjarnardottir, A. Palsdottir, G. O. Hreggvidsson, J. K. Kristjansson, O. Hoist and G. Eggertsson (1998). “Cloning, sequencing and overexpression of a Rhodothermus marinus gene encoding a thermostable cellulase of glycosyl hydrolase family 12.” Appl Microbiol Biotechnol 49(3): 277-284.
Hamada, N., K. Ishikawa, N. Fuse, R. Kodaira, M. Shimosaka, Y. Amano, T. Kanda and M. Okazaki (1999). “Purification, characterization and gene analysis of exo-cellulase II (Ex-2) from the white rot basidiomycete Irpex lacteus.” J Biosci Bioeng 87(4): 442-451.
Hasper, A. A., E. Dekkers, M. van Mil, P. J. van de Vondervoort and L. H. de Graaff (2002). “EglC, a new endoglucanase from Aspergillus niger with major activity towards xyloglucan.” Appi Environ Microbiol 68(4): 1556-1560.
Hilden, K. S., M. R. Makela, T. K. Hakala, A. Hatakka and T. Lundell (2006). “Expression on wood, molecular cloning and characterization of three lignin peroxidase (LiP) encoding genes of the white rot fungus Phlebia radiata.” Curr Genet 49(2): 97-105.
Homrich, M. S., B. Wiebke-Strohm, R. L. M. Weber and M. H. Bodanese-Zanettini (2012). “Soybean genetic transformation: A valuable tool for the functional study of genes and the production of agronomically improved plants.” Genetics and Molecular Biology 35(4): 998-1010.
Hood, E. E. (2004). “Where, oh where has my protein gone?“Trends in Biotechnology 22(2): 53-55.
Howard, J. A. and E. Hood (2005). “Bioindustrial and biopharmaceutical products produced in plants.” Advances in Agronomy, Vol 85 85: 91-124.
Howard, R. L. (2005). “Refolding and characterisation of a heterologous expressed Phanerochaete chrysosporium cellobiohydrolase (CBH1.2).” African Journal of Biotechnology 4(10): 1185-1188.
Hudson, L. C., K. L. Bost and K. J. Piller (2011). Optimizing recombinant protein expression in Soybean. Soybean: Molecular Aspects of Breeding. A. Sudaric, InTech: 19-42.
Irwin, D. C., S. Zhang and D. B. Wilson (2000). “Cloning, expression and characterization of a family 48 exocellulase, Cel48A, from Thermobifida fusca.” Eur J Biochem 267(16): 4988-4997.
Jeoh, T., W. Michener, M. E. Himmel, S. R. Decker and W. S. Adney (2008). “Implications of cellobiohydrolase glycosylation for use in biomass conversion.” Biotechnol Biofuels 1(1): 10.
Jung, S. K., V. Parisutham, S. H. Jeong and S. K. Lee (2012). “Heterologous expression of plant cell wall degrading enzymes for effective production of cellulosic biofuels.” J Biomed Biotechnol 2012: 405842.
Kanamasa, S., T. Kawaguchi, G. Takada, S. Kajiwara, J. Sumitani and M. Arai (2007). “Development of an efficient production method for beta-mannosidase by the creation of an overexpression system in Aspergillus aculeatus.” Lett Appl Microbiol 45(2): 142-147.
Kapp, K., S. Schrempf, M. K. Lemberg and B. Dobberstein (2009). Post-targeting functions of signal peptides. Protein transport into the endoplasmic reticulum. R. Zimmerman. Austin, Tex., Landes Bioscience.
Karnaouri, A., E. Topakas, T. Paschos, I. Taouki and P. Christakopoulos (2013). “Cloning, expression and characterization of an ethanol tolerant GH3 beta-glucosidase from Myceliophthora thermophila.” PeerJ 1: e46.
Kiarie, E., L. F. Romero and C. M. Nyachoti (2013). “The role of added feed enzymes in promoting gut health in swine and poultry.” Nutr Res Rev 26(1): 71-88.
Kim, S. J., J. A. Lee, J. C. Joo, Y. J. Yoo, Y. H. Kim and B. K. Song (2010). “The development of a thermostable CiP (Coprinus cinereus peroxidase) through in silico design.” Biotechnol Prog 26(4): 1038-1046.
Kim, S. J., J. A. Lee, Y. H. Kim and B. K. Song (2009). “Optimization of the functional expression of Coprinus cinereus peroxidase in Pichia pastoris by varying the host and promoter.” J Microbiol Biotechnol 19(9): 966-971.
La Grange, D. C., I. S. Pretorius, M. Claeyssens and W. H. van Zyl (2001). “Degradation of xylan to D-xylose by recombinant Saccharomyces cerevisiae coexpressing the Aspergillus niger beta-xylosidase (xlnD) and the Trichoderma reesei xylanase II (xyn2) genes.” Appl Environ Microbiol 67(12): 5512-5519.
Lee, M. H. and S. W. Lee (2013). “Bioprospecting potential of the soil metagenome: novel enzymes and bioactivities.” Genomics Inform 11(3): 114-120.
Li, D., N. Li, B. Ma, M. B. Mayfield and M. H. Gold (1999). “Characterization of genes encoding two manganese peroxidases from the lignin-degrading fungus Dichomitus squalens (1).” Biochim Biophys Acta 1434(2): 356-364.
Li, N., P. Shi, P. Yang, Y. Wang, H. Luo, Y. Bai, Z. Zhou and B. Yao (2009). “A xylanase with high pH stability from Streptomyces sp. S27 and its carbohydrate-binding module with/without linker-region-truncated versions.” Appl Microbiol Biotechnol 83(1): 99-107.
Luo, H., J. Li, J. Yang, H. Wang, Y. Yang, H. Huang, P. Shi, T. Yuan, Y. Fan and B. Yao (2009). “A thermophilic and acid stable family-10 xylanase from the acidophilic fungus Bispora sp. MEY-1.” Extremophiles 13(5): 849-857.
Luo, H., Y. Wang, H. Wang, J. Yang, Y. Yang, H. Huang, P. Yang, Y. Bai, P. Shi, Y. Fan and B. Yao (2009). “A novel highly acidic beta-mannanase from the acidophilic fungus Bispora sp. MEY-1: gene cloning and overexpression in Pichia pastoris.” Appl Microbiol Biotechnol 82(3): 453-461.
Mahadevan, S. A., S. G. Wi, Y. O. Kim, K. H. Lee and H. J. Bae (2011). “In planta differential targeting analysis of Thermotoga maritima Cel5A and CBM6-engineered Cel5A for autohydrolysis.” Transgenic Res 20(4): 877-886.
Margeot, A., B. Hahn-Hagerdal, M. Edlund, R. Slade and F. Monot (2009). “New improvements for lignocellulosic ethanol.” Curr Opin Biotechnol 20(3): 372-380.
Merino, S. T. and J. Cherry (2007). “Progress and challenges in enzyme development for biomass utilization.” Adv Biochem Eng Biotechnol 108: 95-120.
Miki, Y., M. Morales, F. J. Ruiz-Duenas, M. J. Martinez, H. Wariishi and A. T. Martinez (2009). “Escherichia coli expression and in vitro activation of a unique ligninolytic peroxidase that has a catalytic tyrosine residue.” Protein Expr Purif 68(2): 208-214.
Miyazaki, K. (2005). “A hyperthermophilic laccase from Thermus thermophilus HB27.” Extremophiles 9(6): 415-425.
Mohanram, S., D. Amat, J. Choudhary, A. Arora and L. Nain (2013). “Novel perspectives for evolving enzymes cocktails for lignocellulose hydrolysis in biorefineries.” Sustainable Chemical Processes 1(1): 15-27.
Mohorcic, M., M. Bencina, J. Friedrich and R. Jerala (2009). “Expression of soluble versatile peroxidase of Bjerkandera adusta in Escherichia coli.” Bioresour Technol 100(2): 851-858.
Nielsen, N. C., C. D. Dickinson, T. J. Cho, V. H. Thanh, B. J. Scallon, R. L. Fischer, T. L. Sims, G. N. Drews and R. B. Goldberg (1989). “Characterization of the Glycinin Gene Family in Soybean.” Plant Cell 1(3): 313-328.
O'Callaghan, J., M. M. O'Brien, K. McClean and A. D. Dobson (2002). “Optimisation of the expression of a Trametes versicolor laccase gene in Pichia pastoris.” J Ind Microbiol Biotechnol 29(2): 55-59.
Oakes, J. L., K. L. Bost and K. J. Piller (2009). “Stability of a soybean seed-derived vaccine antigen following long-term storage, processing and transport in the absence of a cold chain.” Journal of the Science of Food and Agriculture 89(13): 2191-2199.
Oraby, H., B. Venkatesh, B. Dale, R. Ahmad, C. Ransom, J. Oehmke and M. Sticklen (2007). “Enhanced conversion of plant biomass into glucose using transgenic rice-produced endoglucanase for cellulosic ethanol.” Transgenic Res 16(6): 739-749.
Park, C. S., T. Kawaguchi, J. Sumitani, G. Takada, K. Izumori and M. Arai (2005). “Cloning and sequencing of an exoglucanase gene from Streptomyces sp. M 23, and its expression in Streptomyces lividans TK-24.” J Biosci Bioeng 99(4): 434-436.
Pauly, M., L. N. Andersen, S. Kauppinen, L. V. Kofod, W. S. York, P. Albersheim and A. Darvill (1999). “A xyloglucan-specific endo-beta-)1,4-glucanase from Aspergillus aculeatus: expression cloning in yeast, purification and characterization of the recombinant enzyme.” Glycobiology 9(1): 93-100.
Paz, M. M., J. C. Martinez, A. B. Kalvig, T. M. Fonger and K. Wang (2006). “Improved cotyledonary node method using an alternative explant derived from mature seed for efficient Agrobacterium-mediated soybean transformation.” Plant Cell Rep 25(3): 206-213.
Petersen, K. and R. Bock (2011). “High-level expression of a suite of thermostable cell wall-degrading enzymes from the chloroplast genome.” Plant Mol Biol 76(3-5): 311-321.
Piller, K. J., T. E. Clemente, S. M. Jun, C. C. Petty, S. Sato, D. W. Pascual and K. L. Bost (2005). “Expression and immunogenicity of an Escherichia coli K99 fimbriae subunit antigen in soybean.” Planta 222(1): 6-18.
Powell, R., L. C. Hudson, K. C. Lambirth, D. Luth, K. Wang, K. L. Bost and K. J. Piller (2011). “Recombinant expression of homodimeric 660 kDa human thyroglobulin in soybean seeds: an alternative source of human thyroglobulin.” Plant Cell Reports 30(7): 1327-1338.
Rasmussen, L. E., H. R. Sorensen, J. Vind and A. Vikso-Nielsen (2006). “Mode of action and properties of the beta-xylosidases from Talaromyces emersonii and Trichoderma reesei.” Biotechnol Bioeng 94(5): 869-876.
Ravindran, V. and J. H. Son (2011). “Feed enzyme technology: present status and future developments.” Recent Pat Food Nutr Agric 3(2): 102-109.
Record, E., P. J. Punt, M. Chamkha, M. Labat, C. A. van Den Hondel and M. Asther (2002). “Expression of the Pycnoporus cinnabarinus laccase gene in Aspergillus niger and characterization of the recombinant enzyme.” Eur J Biochem 269(2): 602-609.
Rodriguez Couto, S. and J. L. Toca Herrera (2006). “Industrial and biotechnological applications of laccases: a review.” Biotechnol Adv 24(5): 500-513.
Rodriguez, E., F. J. Ruiz-Duenas, R. Kooistra, A. Ram, A. T. Martinez and M. J. Martinez (2008). “Isolation of two laccase genes from the white-rot fungus Pleurotus eryngii and heterologous expression of the pel3 encoded protein.” J Biotechnol 134(1-2): 9-19.
Ruiz-Duenas, F. J., S. Camarero, M. Perez-Boada, M. J. Martinez and A. T. Martinez (2001). “A new versatile peroxidase from Pleurotus.” Biochem Soc Trans 29(Pt 2): 116-122.
Ruiz-Duenas, F. J., M. J. Martinez and A. T. Martinez (1999). “Molecular characterization of a novel peroxidase isolated from the ligninolytic fungus Pleurotus eryngii.” Mol Microbiol 31(1): 223-235.
Sainz, M. B. (2009). “Commercial cellulosic ethanol: The role of plant-expressed enzymes.” In Vitro Cellular & Developmental Biology-Plant 45(3): 314-329.
Salame, T. M., D. Knop, D. Levinson, S. J. Mabjeesh, O. Yarden and Y. Hadar (2014). “Inactivation of a Pleurotus ostreatus versatile peroxidase-encoding gene (mnp2) results in reduced lignin degradation.” Environ Microbiol 16(1): 265-277.
Shao, W., Y. Xue, A. Wu, I. Kataeva, J. Pei, H. Wu and J. Wiegel (2011). “Characterization of a novel beta-xylosidase, XylC, from Thermoanaerobacterium saccharolyticum JW/SL-YS485.” Appi Environ Microbiol 77(3): 719-726.
Shen, B., X. Sun, X. Zuo, T. Shilling, J. Apgar, M. Ross, O. Bougri, V. Samoylov, M. Parker. E. Hancock, H. Lucero, B. Gray, N. A. Ekborg, D. Zhang, J. C. Johnson, G. Lazar and R. M. Raab (2012). “Engineering a thermoregulated intein-modified xylanase into maize for consolidated lignocellulosic biomass processing” Nat Biotechnol 30(11): 1131-1136.
Smith, T. L., H. Schalch, J. Gaskell, S. Covert and D. Cullen (1988). “Nucleotide sequence of a ligninase gene from Phanerochaete chrysosporium.” Nucleic Acids Res 16(3): 1219.
Sticklen, M. B. (2008). “Plant genetic engineering for biofuel production: towards affordable cellulosic ethanol.” Nat Rev Genet 9(6): 433-443.
Sundaramoorthy, M., M. H. Gold and T. L. Poulos (2010). “Ultrahigh (0.93 A) resolution structure of manganese peroxidase from Phanerochaete chrysosporium: implications for the catalytic mechanism.” J Inorg Biochem 104(6): 683-690.
Sundaramoorthy, M., K. Kishi, M. H. Gold and T. L. Poulos (1994). “The crystal structure of manganese peroxidase from Phanerochaete chrysosporium at 2.06-A resolution.” J Biol Chem 269(52): 32759-32767.
Taylor, L. E., 2nd, Z. Dai, S. R. Decker, R. Brunecky, W. S. Adney, S. Y. Ding and M. E. Himmel (2008). “Heterologous expression of glycosyl hydrolases in planta: a new departure for biofuels.” Trends Biotechnol 26(8): 413-424.
Te'o, V. S., D. J. Saul and P. L. Bergquist (1995). “celA, another gene coding for a multidomain cellulase from the extreme thermophile Caldocellum saccharolyticum.” Appi Microbiol Biotechnol 43(2): 291-296.
Trick, H. N., R. D. Dinkins, E. R. Santarem, R. Di, V. Samoylov, C. A. Meurer, D. R. Walker, W. A. Parrott, J. J. Finer and G. B. Collins (1997). “Recent Advances in soybean transformation.” Plant Tissue Culture and Biotechnology 3(1): 1-26.
Ufot, U. F. and M. I. Akpanabiatu (2012). “An engineered Phlebia radiata manganese peroxidase: Expression, folding, purification, and preliminary characterization.” American Journal of Molecular Biology 2: 359-370.
Ussery, D. W. and P. F. Hallin (2004). “Genome Update: AT content in sequenced prokaryotic genomes.” Microbiology-Sgm 150: 749-752.
van den Brink, J. and R. P. de Vries (2011). “Fungal enzyme sets for plant polysaccharide degradation.” Appl Microbiol Biotechnol 91(6): 1477-1492.
Varela, E., B. Bockle, A. Romero, A. T. Martinez and M. J. Martinez (2000). “Biochemical characterization, cDNA cloning and protein crystallization of aryl-alcohol oxidase from Pleurotus pulmonarius.” Biochim Biophys Acta 1476(1): 129-138.
Varela, E., A. T. Martinez and M. J. Martinez (1999). “Molecular cloning of aryl-alcohol oxidase from the fungus Pleurotus eryngii, an enzyme involved in lignin degradation.” Biochem J 341 (Pt 1): 113-117.
Viikari, L., M. Alapuranen, T. Puranen, J. Vehmaanpera and M. Siika-Aho (2007). “Thermostable enzymes in lignocellulose hydrolysis.” Adv Biochem Eng Biotechnol 108: 121-145.
Voutilainen, S. P., T. Puranen, M. Siika-Aho, A. Lappalainen, M. Alapuranen, J. Kallio, S. Hooman, L. Viikari, J. Vehmaanpera and A. Koivula (2008). “Cloning, expression, and characterization of novel thermostable family 7 cellobiohydrolases.” Biotechnol Bioeng 101(3): 515-528.
Waters, D. M., L. A. Ryan, P. G. Murray, E. K. Arendt and M. G. Tuohy (2011). “Characterisation of a Talaromyces emersonii thermostable enzyme cocktail with applications in wheat dough rheology.” Enzyme Microb Technol 49(2): 229-236.
Xie, J., L. Feng, N. Xu, G. Zhu, J. Yang, X. Xiaoli and S. Fu (2007). “Studies on the fusion of lignolytic enzyme cDNAs and their expression.” BioResources 2(4): 598-604.
Yeoman, C. J., Y. Han, D. Dodd, C. M. Schroeder, R. I. Mackie and I. K. Cann (2010). “Thermostable enzymes as biocatalysts in the biofuel industry.” Adv Appl Microbiol 70: 1-55.
Yi, X., Y. Shi, H. Xu, W. Li, J. Xie, R. Yu, J. Zhu, Y. Cao and D. Qiao (2010). “Hyperexpression of two Aspergillus Niger Xylanase Genes in Escherichia Coli and Characterization of the Gene Products.” Braz J Microbiol 41(3): 778-786.
Zhang, Y., X. Xu, X. Zhou, R. Chen, P. Yang, Q. Meng, K. Meng, H. Luo, J. Yuan, B. Yao and W. Zhang (2013). “Overexpression of an acidic endo-beta-1,3-1,4-glucanase in transgenic maize seed for direct utilization in animal feed.” PLoS One 8(12): e81993.
Zhang, Z., A. A. Donaldson and X. Ma (2012). “Advancements and future directions in enzyme technology for biomass conversion.” Biotechnol Adv 30(4): 913-919.
Zhang, Z. Y., A. Q. Xing, P. Staswick and T. E. Clemente (1999). “The use of glufosinate as a selective agent in Agrobacterium-mediated transformation of soybean.” Plant Cell Tissue and Organ Culture 56(1): 37-46.
Zverlov, V., S. Mahr, K. Riedel and K. Bronnenmeier (1998). “Properties and gene structure of a bifunctional cellulolytic enzyme (CelA) from the extreme thermophile ‘Anaerocellum thermophilum’ with separate glycosyl hydrolase family 9 and 48 catalytic domains.” Microbiology 144 (Pt 2): 457-465.
Zverlov, V. V., G. A. Velikodvorskaya and W. H. Schwarz (2002). “A newly described cellulosomal cellobiohydrolase, CelO, from Clostridium thermocellum: investigation of the exo-mode of hydrolysis, and binding capacity to crystalline cellulose.” Microbiology 148(Pt 1): 247-255.

We claim:

1. A composition comprising a transgenic soy plant that has been transformed with one or more genes that expresses one or more enzymes, said one or more enzymes being capable of at least partially metabolizing lignin, hemicellulose, and/or cellulose wherein the one or more enzymes is/are present at a concentration of at least 2 g/800 g of soy powder without additional concentration, filtration, or lyophilization.

2. The composition of claim 1, wherein the one or more enzymes is/are one or more members selected from the group consisting of laccases, peroxidases, xylanases, endoglucanases, cellulases, and glucosidases.

3. The composition of claim 2, wherein the one or more enzymes is present at a concentration of at least 4 g/800 g of soy powder.

4. The composition of claim 2, wherein the composition is in a powder form, wherein 90% of the powder has a particle size of about 5 to 860 microns.

5. The composition of claim 1, wherein said composition comprises at least a first enzyme and a second enzyme, said first enzyme and said second enzyme being capable of at least partially metabolizing lignin, hemicellulose, and/or cellulose.

6. The composition of claim 5, wherein the first enzyme and the second enzyme are one or more members selected from the group consisting of laccases, peroxidases, xylanases, endoglucanases, cellulases, and glucosidases.

7. The composition of claim 5, wherein the composition is in a powder form, wherein 90% of the powder has a particle size of about 5 to 860 microns.

8. A powder derived from soy seed, wherein said powder comprises a transgenic soy plant that has been transformed with a gene that expresses an enzyme, said enzyme being capable of at least partially metabolizing cellulose, lignin, and/or hemicellulose, said powder being of a size between about 5-860 micrometers to facilitate dissolution.

9. The powder of claim 8, wherein said enzyme is one or more members selected from the group consisting of laccases, peroxidases, xylanases, endoglucanases, cellulases, and glucosidases.

10. The powder of claim 8, wherein the enzyme retains at least 80% activity relative to freshly expressed enzyme after about one year at room temperature.

11. The powder of claim 8, made from a transgenic soy plant, said powder comprising an enzyme that is capable of at least partially metabolizing cellulose, lignin, and/or hemicellulose, said powder made by transforming a soy plant with a gene that expresses said enzyme, expressing said enzyme in said soy plant to generate a soy plant with an expressed enzyme, micronizing said soy plant with said expressed enzyme until it is a size that is about 5-860 micrometers, wherein said powder containing said enzyme is present at a concentration of at least 2 g enzyme/800 g of soy powder without additional concentration, filtration, or lyophilization, and wherein said powder is in a form that allows said enzyme to remain functional at room temperature for a period of at least 12 months with less than 20% loss of enzymatic activity.

12. The powder of claim 11, wherein the powder is derived from one or more soy seeds.

13. The powder of claim 11, wherein the enzyme is present at a concentration of at least 4 g enzyme/800 g of soy powder without additional concentration, filtration, or lyophilization.

14. The powder of claim 11, wherein the enzyme is present at a concentration of at least 6 g enzyme/800 g of soy powder without additional concentration, filtration, or lyophilization.

15. A transgenic soy product that is in powder or flake form, said soy product comprising an overexpressed enzyme, said soy product being comprised of at least a first harvest and a second harvest wherein a variance between enzyme activity from the first harvest and the second harvest is less than about 10%.

16. The transgenic soy product of claim 15, wherein the enzyme is one or more members selected from the group consisting of laccases, peroxidases, xylanases, endoglucanases, cellulases, and glucosidases.

17. A method of making ethanol comprising adding the composition of claim 1 to a plant or a partially metabolized plant.

18. A method of at least partially metabolizing cellulose, lignin, and/or hemicellulose, comprising treating said cellulose, lignin, and/or hemicellulose with the powder of claim 8, wherein said powder is derived from a transgenic soy plant, said transgenic soy plant being transformed with a gene that expresses an enzyme that is capable of at least partially metabolizing cellulose, lignin, and/or hemicellulose.

19. The method of claim 16, wherein the enzyme is one or more members selected from the group consisting of laccases, peroxidases, xylanases, endoglucanases, cellulases, and glucosidases.

20. The method of claim 18, wherein the powder is of a size that has 90% in a range between about 5 and 200 microns.


SEQUENCE LISTING (65 sequences)

SEQ ID NO: 1 - protein, 519 aa, Trametes versicolor
MGLQRFSFFVTLALVARSLAAIGPVASFVVANAPVSPDGFLRDAIVVNGVVPSPLIRAKKGDRFQLNVVDTLTNHSMLKSTSIHWHGFFQAGTNWADGPAFVNQCPIASGHSFLYDFHVPDQAGTFWYHSHLSTQYCDGLRGPFVVYDPKDPHASRYDVDNESTVITLTDWYHTAARLGPRFPLGADATVINGLGRSASTPTAALAVINVQHGKRYRFRLVSISCDPNYTFSIDGHNLTVIEVDGINSQPLLVDSIQIFAAQRYSFVLNANQTVGNYWVRANPNFGTVGFAGGINSAILRYQGAPVAEPTTTQTPSVIPLIETNLHPLARMPVPGTRTPGGVDKALKLAFNFNGTNFFINNASFTPPTVPVLLQILSGAQTAQELLPAGSVYPLPAHSTIEITLPATALAPGAPHPFHLHGHAFAVVRSAGSTTYNYNDPIFRDVVSTGTPAAGDNVTIRFQTDNLGPWFLHCHIDFHLEAGFAIVFAEDVADVKAANPVPKAWSDLCPIYDGLSEADQ

SEQ ID NO: 2 - protein, 616 aa, Myceliophthora thermophila
MKSFISAATLLVGILTPSVAAAPPSTPEQRDLLVPITEREEAAVKARQQSCNTPSNRACWTDGYDINTDYEVDSPDTGVVRPYTLTLTEVDNWTGPDGVVKEKVMLVNRPTIFADWGDTIQVTVINNLETNGTSIHWHGLHQKGTNLHDGANGITECPIPPKGGRKVYRFKAQQYGTSWYHSHFSAQYGNGVVGAIQINGPASLPYDTDLGVFPISDYYYSSADELVELTKNSGAPFSDNVLFNGTAKHPETGEGEYANVTLTPGRRHRLRLINTSVENHFQVSLVNHTMTIIAADMVPVNAMTVDSLFLGVGQRYDVVIEASRTPGNYWFNVTFGGGLLCGGSRNPYPAAIFHYAGAPGGPPTDEGKAPVDHNCLDLPNLKPVVARDVPLSGFAKRPDNTLDVTLDTTGTPLFVWKVNGSAINIDWGRPVVDYVLTQNTSFPPGYNIVEVNGADQWSYWLIENDPGAPFTLPHPMHLHGHDFYVLGRSPDESPASNERHVFDPARDAGLLSGANPVRRDVTMLPAFGWVVLAFRADNPGAWLFHCHIAWHVSGGLGVVYLERADDLRGAVSDADADDLDRLCADWRHYWPTNPYPKSDSGLKHRWVEEGEWLVKA

SEQ ID NO: 3 - protein, 533 aa, Pleurotus eryngii
MAVAFIALVSLALALVRVEASIGPRGTLNIANEVIKPDGFSRSAVLAGGSYPGPLIKGETGDRFQINVVNKLADTSMPVDTSIHWHGIFVRGHNWADGPAMVTQCPIVPGHSFLYDFEIPDQAGTFWYHSHLGTQYCDGLRGPFVVYSKNDPHKRLYDVDDESTVLTVGDWYHAPSLSLSGVPHPDSTLFNGLGRSLNGPASPLYVMNVVKGKRYRIRLINTSCDSNYQFSIDGHAFTVIEADGENTQPLQVDQVQIFAGQRYSLVLNANQAVGNYWIRANPNSGDPGFANQMNSAILRYKGARNVDPTTPERNATNPLREYNLRPLIKEPAPGKPFPGGADHNINLNFAFDPATVLFTANNYTFVPPTVPVLLQILSGTRDAHDLAPAGSIYDIKLGDVVEVTMPALVFAGPHPMHLHGHSFAVVRSAGSSTYNYENPVRRDVVSIGDDPTDNVTIRFVADNAGPWFLHCHIDWHLDLGFAVVFAEGVNQTAVANPVPEAWNDLCPIYNSSNPSKLLMGTNAIGRLPAPLKA

SEQ ID NO: 4 - protein, 518 aa, Pycnoporus cinnabarinus
MSRFQSLFFFVLVSLTAVANAAIGPVADLTLTNAQVSPDGFAREAVVVNGITPAPLITGNKGDRFQLNVIDQLTNHTMLKTSSIHWHGFFQQGTNWADGPAFVNQCPIASGHSFLYDFQVPDQAGTFWYHSHLSTQYCDGLRGPFVVYDPNDPHASLYDIDNDDTVITLADWYHVAAKLGPRFPFGSDSTLINGLGRTTGIAPSDLAVIKVTQGKRYRFRLVSLSCDPNHTFSIDNHTMTIIEADSINTQPLEVDSIQIFAAQRYSFVLDASQPVDNYWIRANPAFGNTGFAGGINSAILRYDGAPEIEPTSVQTTPTKPLNEVDLHPLSPMPVPGSPEPGGVDKPLNLVFNFNGTNFFINDHTFVPPSVPVLLQILSGAQAAQDLVPEGSVFVLPSNSSIEISFPATANAPGFPHPFHLHGHAFAVVRSAGSSVYNYDNPIFRDVVSTGQPGDNVTIRFETNNPGPWFLHCHIDFHLDAGFAVVMAEDTPDTKAANPVPQAWSDLCPIYDALDPSDL

SEQ ID NO: 5 - protein, 462 aa, Thermus thermophilus
MLARRSFLQAAAGSLVLGLARAQGPSFPEPKVVRSQGGLLSLKLSATPTPLALAGQRATLLTYGGSFPGPTLRVRPRDTVRLTLENRLPEPTNLHWHGLPISPKVDDPFLEIPPGESWTYEFTVPKELAGTFWYHPHLHGRVAPQLFAGLLGALVVESSLDAIPELREAEEHLLVLKDLALQGGRPAPHTPMDWMNGKEGDLVLVNGALRPTLVAQKATLRLRLLNASNARYYRLALQDHPLYLIAADGGFLEEPLEVSELLLAPGERAEVLVRLRKEGRFLLQALPYDRGAMGMMDMGGMAHAMPQGPSRPETLLYLIAPKNPKPLPLPKALSPFPTLPAPVVTRRLVLTEDMMAARFFINGQVFDHRRVDLKGQAQTVEVWEVENQGDMDHPFHLHVHPFQVLSVGGRPFPYRAWKDVVNLKAGEVARLLVPLREKGRTVFHCHIVEHEDRGMMGVLEVG

SEQ ID NO: 6 - protein, 363 aa, Coprinus cinereus
MKLSLLSTFAAVIIGALALPQGPGGGGSVTCPGGQSTSNSQCCVWFDVLDDLQTNFYQGSKCESPVRKILRIVFHDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP

SEQ ID NO: 7 - protein, 372 aa, Phanerochaete chrysosporium
MAFKQLFAAISLALLLSAANAAAVIEKRATCSNGKTVGDASCCAWFDVLDDIQQNLFHGGQCGAEAHESIRLVFHDSIAISPAMEAQGKFGGGGADGSIMIFDDIETAFHPNIGLDEIVKLQKPFVQKHGVTPGDFIAFAGAVALSNCPGAPQMNFFTGRAPATQPAPDGLVPEPFHTVDQIINRVNDAGEFDELELVWMLSAHSVAAVNDVDPTVQGLPFDSTPGIFDSQFFVETQLRGTAFPGSGGNQGEVESPLPGEIRIQSDHTIARDSRTACEWQSFVNNQSKLVDDFQFIFLALTQLGQDPNAMTDCSDVIPQSKPIPGNLPFSFFPAGKTIKDVEQACAETPFPTLTTLPGPETSVQRIPPPPGA

SEQ ID NO: 8 - protein, 361 aa, Trametes cervina
MAFQTLFALATLATTVLAVPSPLVSCGGGRSVKNAACCAWFPVLDDIQANLFNGGKCEEEAHEAVRLTFHDAVGFSLAAQKAGKFGGGGADGSILAFSDIETAFIPNFGLEFTTEGFIPFALAHGVSFGDFVQFAGAVGAANCAGGPRLQFLAGRSNISQPSPDGLVPDPTDSADKILARMADIGFSPTEVVHLLASHSIAAQYEVDTDVAGSPFDSTPSVFDTQFFVESLLHGTQFTGSGQGGEVMSPIPGEFRLQSDFALSRDPRTACEWQALVNNQQAMVNNFEAVMSRLAVIGQIPSELVDCSDVIPTPPLAKVAQVGSLPPGKSMADVQVACTNGMPFPSLPTSPGPVQTVAPVLG

SEQ ID NO: 9 - protein, 382 aa, Phlebia radiata
MAFGSLLAFVALAAITRAAPTAESAVCPDGTRVTNAACCAFIPLAQDLQETLFQGDCGEDAHEVIRLTFHDAIAISQSLGPQAGGGADGSMLHFPTIEPNFSANNGIDDSVNNLLPFMQKHDTISAADLVQFAGAVALSNCPGAPRLEFMAGRPNTTIPAVEGLIPEPQDSVTKILQRFEDAGNFSPFEVVSLLASHTVARADKVDETIDAAPFDSTPFTFDTQVFLEVLLKGTGFPGSNNNTGEVMSPLPLGSGSDTGEMRLQSDFALARDERTACFWQSFVNEQEFMAASFKAAMAKLAILGHSRSSLIDCSDVVPVPKPAVNKPATFPATKGPKDLDTLTCKALKFPTLTSDPGATETLIPHCSNGGMSCPGVQFDGPA

SEQ ID NO: 10 - protein, 395 aa, Phanerochaete chrysosporidium
MAFKWSSILALVTLATLASAAPTQSTVTCSDGTVVPDSVCCEFIPLREALNDQVIQSDCGEDAHELLRLTFHDAIAISQSLGPSAGGGADGSMLLFPTVEPAFFANLGIADSVNNLIPFMSQFPNISPGDLVQFAGAVAITNCPGAPQLEFLAGRPNGTAPAIDGLIPEPQDSIDDILARFDDAGGFTPFEVVSLLASHTVARADHVDPTLDAAPFDSTPFTFDTQIFLEVLLKGTGFPGTDNNTGEVASPIPVTNGTDVGELRLQSDFGLAHDSRTACFWQGFVNQQDFMAQSFKAAMAKLAVLGHNAADLVNCSAVIPTPLPATGKPATFPATLGPDDLELSCTTEPFPSLTTDPGAQETLIPHCSDGSMDCESVQFDGPATNFGGDDDDDDS

SEQ ID NO: 11 - protein, 390 aa, Dichomitus squalens
MAFKLWSMLPLVALATVAVAAPSRQTVCSDGTVVPDSVCCEFVPLAQALQKEVLMGDCGEDAHELLRLTFHDAIAISRSKGPSAGGGADGSMLIFPTVEPAFFANLGIADSVNNLIPFLSQFPKISAGDLVQFAGAVAVGNCPGAPQLEFRAGRPNATAPAIEGLIPEPQNNITEILERFDDAGGFSPFEVVSLLASHTVARADHVDPTLDAAPFDSTPFTFDTQIFLEVLLKGVGFPGTGNNTGEVSSPLPVSSGTDVGELRLQSDFGLAHDERTACFWQGFVNEQEFMAQSFKAAMAKLAVLGHNADDLVDCSAVVPKPKPATGKPASFPATKGPKDLELSCTSKKFPTLTTDHGAQETLIPHCSNGSMNCTTVQFDGPATNFDGDDS

SEQ ID NO: 12 - protein, 361 aa, Dichomitus squalens
MTFASLSALVLVFAVTVQVAQAVSLPQKRATCAGGQVTANAACCVLFPLMEDLQKNLFDDGACGEDAHEALRLTFHDAIGFSPSRGVMGGADGSVITFSDTEVNFPANLGIDEIVEAEKPFLARHNISAGDLVHFAGTLAVTNCPGAPRIPFFLGRPPAKAASPIGLVPEPFDTITDILARMDDAGFVSVEVVWLLSAHSVAAADHVDETIPGTPFDSTPNLFDSQIFIETQLRGISFPGTGGNHGEVQSPLKGEMRLQSDHLFARDDRTSCEWQSMTNDQQKIQDRFSDTLFKMSMLGQNQDAMIDCSDVIPVPAALVTKPHLPAGKSKTDVEQACATGAFPALGADPGPVTSVPRVPPA

SEQ ID NO: 13 - protein, 366 aa, Pleurotus ostreatus
MAFAKLSALVLALGATVALGAPSLNKRVTCATGQTTANEACCALFPILDDIQTNLFDGAQCGEEVHESLRLTFHDAIAFSPALTNAGQFGGGGADGSMIIFSDTEPNFHANLGIDEIVEAQKPFIARHNICAADFIQFAGAIGVSNCAGAPRLNFFLGRPDATQIPPDGLVPEPFDDVTKILSRMGDAGFSTVEVVWLLSSHTIAAADLVDPSIPGTPFDSTPSTFDSQFFLETMLQGTAFPGTPGNQGEVESPLAGEMRLQSDFLLARDSRSACEWQSMVNNMPKIQNRFTQVMKKLSLLGHNQADLIDCSDVIPVPKTLTKAATFPAGKSQADVEIVCNAAATPFPALSSDPGPVTAVPPVPPS

SEQ ID NO: 14 - protein, 345 aa, Pleurotus ostreatus
AITRRVACLDGVNTATNAACCALFAVRDDIQQNLFDGGECGEEVHESLRLTFHDAIGISPSLAATGKFGGGGADGSIMIFDDIEPNFHANNGVDEIINAQKPFVAKHNMTAGDFIQFAGAVGVSNCPGAPQLSFFLGRPAATQPAPDGLVPEPFDSVTDILNRFADAGGFTTQEVVWLLASHSIAAADHVDPTIPGSPFDSTPEIFDTQFFVETLLKGTLFPGTSGNQGEVESPLAGEIRLQSDADFARDSRTACEWQSFVNNQPRMQVLFKAAMQKLSILGHDLTQMIDCSDVIPVPPSTAVRGSHLPAGNTLDDIEQACASTPFPSLTADPGPATSVAPVPPS

SEQ ID NO: 15 - protein, 361 aa, Bjerkandera adusta
MSFKTLSALALALGAAVQFASAAVPLVQKRATCADGRTTANAACCVLFPILDDIQENLFDGAQCGEEVHESLRLTFHDAIGFSPTLGGGGADGSIIAFDTIETNFPANAGIDEIVSAQKPFVAKHNISAGDFIQFAGAVGVSNCPGGVRIPFFLGRPDAVAASPDHLVPEPFDSVDSILARMSDAGFSPVEVVWLLASHSIAAADKVDPSIPGTPFDSTPGVFDSQFFIETQLKGRLFPGTADNKGEAQSPLQGEIRLQSDHLLARDPQTACEWQSMVNNQPKIQNRFAATMSKMALLGQDKTKLIDCSDVIPTPPALVGAAHLPAGFSLSDVEQACAATPFPALTADPGPVTSVPPVPGS

SEQ ID NO: 16 - protein, 361 aa, Pleurotus eryngii
MSFKTLSALALALGAAVQFASAAVPLVQKRATCDDGRTTANAACCILFPILDDIQENLFDGAQCGEEVHESLRLTFHDAIGFSPTLGGGGADGSIIAFDTIETNFPANAGIDEIVSAQKPFVAKHNISAGDFIQFAGAVGVSNCPGGVRIPFFLGRPDAVAASPDHLVPEPFDSVDSILARMGDAGFSPVEVVWLLASHSIAAADKVDPSIPGTPFDSTPGVFDSQFFIETQLKGRLFPGTADNKGEAQSPLQGEIRLQSDHLLARDPQTACEWQSMVNNQPKIQNRFAATMSKMALLGQDKTKLIDCSDVIPTPPALVGAAHLPAGFSLSDVEQACAATPFPALTADPGPVTSVPPVPGS

SEQ ID NO: 17 - protein, 593 aa, Pleurotus pulmonarius
MSFSALRQLLLIACLALPSLAAANLPTADFDYIVVGAGNAGNVVAARLTEDPNVSVLVLEAGVSDENVLGAEAPLLAPGLVPNSIFDWNYTTTAQAGYNGRSIAYPRGRMLGGSSSVHYMVMMRGSIEDFDRYAAVTGDDGWNWDNIQQFVRKNEMVVPPADNHNTSGEFIPAVHGTNGSVSISLPGFPTPLDDRVLATTQEQSEEFFFNPDMGTGHPLGISWSIASVGNGQRSSSSTAYLRPAQSRPNLSVLINAQVTKLVNSGTTNGLPAFRCVEYAEREGAPTTTVCANKEVVLSAGSVGTPILLQLSGIGDQSDLSAVGIDTIVNNPSVGRNLSDHLLLPATFFVNNNQSFDNLFRDSSEFNADLDQWTNTRTGPLTALIANHLAWLRLPSNSSIFQSVPDPAAGPNSAHWETIFSNQWFHPALPRPDTGNFMSVTNALIAPVARGDIKLATSNPFDKPLINPQYLSTEFDIFTMIQAVKSNLRFLSGQAWADFVIRPFDARLSDPTNDAAIEWNIRDNANTIFHPVGTASMSPRGASWGVVDPDLKVKGVDGLRIVDGSILPFAPNAHTQGPIYLVGERGADLIKADQ

SEQ ID NO: 18 - protein, 666 aa, Aspergillus terreus [sequence omitted]

SEQ ID NO: 19 - protein, 593 aa, Pleurotus eryngii
MSFGALRQLLLIACLALPSLAATNLPTADFDYVVVGAGNAGNVVAARLTEDPDVSVLVLEAGVSDENVLGAEAPLLAPGLVPNSIFDWNYTTTAQAGYNGRSIAYPRGRMLGGSSSVHYMVMMRGSTEDFDRYAAVTGDEGWNWDNIQQFVRKNEMVVPPADNHNTSGEFIPAVHGTNGSVSISLPGFPTPLDDRVLATTQEQSEEFFFNPDMGTGHPLGISWSIASVGNGQRSSSSTAYLRPAQSRPNLSVLINAQVTKLVNSGTTNGLPAFRCVEYAEQEGAPTTTVCAKKEVVLSAGSVGTPILLQLSGIGDENDLSSVGIDTIVNNPSVGRNLSDHLLLPAAFFVNSNQTFDNIFRDSSEFNVDLDQWTNTRTGPLTALIANHLAWLRLPSNSSIFQTFPDPAAGPNSAHWETIFSNQWFHPAIPRPDTGSFMSVTNALISPVARGDIKLATSNPFDKPLINPQYLSTEFDIFTMIQAVKSNLRFLSGQAWADFVIRPFDPRLRDPTDDAAIESYIRDNANTIFHPVGTASMSPRGASWGVVDPDLKVKGVDGLRIVDGSILPFAPNAHTQGPIYLVGKQGADLIKADQ

SEQ ID NO: 20 - protein, 300 aa, Talaromyces emmersonii
MARFSILSTIYLYILFIGSCLAQVPQGSLQQVTNFGDNPTNVGMYVYVPNNLAANPGIVVAIHYCTGSAQAYYSGTPYAQLAEQYGFIVIYPSSPYSGTCWDVSSQAALTHNGGGDSNSIANMVTWTIQQYNADTSKVFVTGSSSGAMMTNVMAATYPELFAAATVYSGVAAGCFVSSTNQVDAWNSSCALGQVIDTPQVWAQVAESMYPGYNGPRPRMQIYHGSADTTLYPQNYQEECKQWAGVFGYDYDSPQQTEPNTPEANYQTTIWGPNLQGIYATGVGHTVPIHGQQDMEWFGFA

SEQ ID NO: 21 - protein, 225 aa, Aspergillis niger
MLTKNLLLCFAAAKAALAVPHDSVAQRSDALHMLSERSTPSSTGENNGFYYSFWTDGGGDVTYTNGDAGAYTVEWSNVGNFVGGKGWNPGSAQDITYSGTFTPSGNGYLSVYGWTTDPLIEYYIVESYGDYNPGSGGTYKGTVTSDGSVYDIYTATRTNAASIQGTATFTQYWSVRQNKRVGGTVTTSNHFNAWAKLGMNLGTHNYQIVATEGYQSSGSSSITVQ

SEQ ID NO: 22 - protein, 424 aa, Bispora sp.
MSFHSLLISGLLASVAVAVPKEAWGITVTETKTVSTTIIATVTELGTCSSTITSPTSDATTTTTSSATNTNPTTTLLATPQPSNWGLNNAARADGKLWFGTAADIPGLEQDDRYYMKEYNNTHDFGGTTPANIMKFMFTEPEQNVFNFTGAQEFLDIAFASHKLVRCHNLIWQSELPTWVTNPTTNWTNETLSKVLQNHVYTLVSHFGDQCYSWDVVNEALSDDPAGSYQNNIWFDTIGPEYVAMAFEYAEKAVKDHKLNVKLYYNDYNIEYPGPKSTAAQNIVKELKARNIQIDGVGLESHFIAGETPSQATQITNMADFTSLDIDVAVTELDVRLYLPPNATSEAQQVADYYATVAACAATERCIGITVWDFDDTYSWVPSTFAGQGYADLFFQPDGPNTPLVKKAAYDGCLQALQHKAESP

SEQ ID NO: 23 - protein, 477 aa, Streptomyces spS27
MGSHALPRSVVRRKLRALLLALVAGVLGVVAALVAPPSAQAAESTLGAAAAQSGRYFGVAIAANRLSDSTYATIAAREFNSVTAENEMKIDATQPQRGQFNFTAADRVYNWAVQNGKEVRGHTLAWHSQQPGWMQNLSGSALRQAMIDHINGVMSHYKGKIAQWDVVNEAFADGSSGARRDSNLQRTGNDWIEVAFRTARAADPSAKLCYNDYNVENWTWAKTQAMYRMVKDFKQRGVPIDCVGFQSHFNSGSPYNSNFRTTLQEFAALGVDVAITELDIQGASPSTYAAVVNDCLAVSRCLGVTVWGVRDSDSWRSEHTPLLFYNNGSKKPAYTAVLDALNGGSTTPPPGDGNTIKGVGSGRCLDVPNASTTDGTQLHLWDCHNGTNQQWTYTNAGELRVYGNKCLDAAGTGNGAKVQIYSCWGGDNQKWRLNSDGSIVGVQSGLCLDAVGAGTANGTLIQLYSCSNGSNQRWTRA

SEQ ID NO: 24 - protein, 213 aa, Bacillus
MFKFTKKFLVGLTAALMSISLFSANASAANTDYWQNWTDGGGTVNAVNGSGGNYSVNWSNTGNFVVGKGWTTGSPFRTINYNAGVWAPNGNAYLTLYGWTRSPLIEYYVVDSWGTYRPTGTYKGTVYSDGGTYDVYTTTRYDAPSIDGDKTTFTQYWSVRQSKRPTGSNATITFSNHVNAWKRYGMNLGSNWSYQVLATEGYQSSGSSNVTVW

SEQ ID NO: 25 - protein, 796 aa, Talaromyces emmersonii
MMTRTAILTALAALLPTATWAQDNQTYANYSSQSQPDLFPRTVATIDLSFPDCENGPLSTNLVCNTSADPWARAEALVSLFTLEELINNTQNTAPGVPRLGLPQYQVWNEALHGLDRANFSDSGEYSWATSFPMPILSMASFNRTLINQIASIIATQARAFNNAGRYGLDSYAPNINGFRSPLWGRGQETPGEDAFFLSSAYAYEYITGLQGGVDPEHVKIVATAKHFAGYDLENWGNVSRLGSNAIITQQDLSEYYTPQFLASARYAKTRSLMCSYNAVNGVPSCSNSFFLQTLLRESFNFVDDGYVSSDCDAVYNVFNPHGYALNQSGAAADSLLAGTDIDCGQTMPWHLNESFYERYVSRGDIEKSLTRLYANLVRLGYFDGNNSVYRNLNWNDVVTTDAWNISYEAAVEGITLLKNDGTLPLSKKVRSIALIGPWANATVQMQGNYYGTPPYLISPLEAAKASGFTVNYAFGTNISTDSTQWFAEAISAAKKSDVIIYAGGIDNTIEAEGQDRTDLKWPGNQLDLIEQLSKVGKPLVVLQMGGGQVDSSSLKANKNVNALVWGGYPGQSGGAALFDILTGKRAPAGRLVSTQYPAEYATQFPANDMNLRPNGSNPGQTYIWYTGTPVYEFGHGLFYTEFQESAAAGTNKTSTLDILDLVPTPHPGYEYIELVPFLNVTVDVKNVGHTPSPYTGLLFANTTAGPKPYPNKWLVGFDRLATIHPAKTAQVTFPVPLGAIARADENGNKVIFPGEYELALNNERSVVVSFSLTGNAATLENWPVWEQAVPGVLQQ

SEQ ID NO: 26 - protein, 638 aa, Thermoanaerobacterium saccharolyticum
MEYHVAKTGSDEGKGTLKDPFLTINKAASVAMAGDTIIVHEGVYREWVKPKYKGLSDKRRITYKAAEGEKVVIKGSERIQSWQRVEGNVWRCQLPNSFFGEFNPYKEEVFGDWLLTVNEKKHLGDVYLNGMSFYEVTNYEDLFNPQLRTEVLDHWTQKIVPIKNAEQTKYVWYAEVDREKTTIYANFQGADPNEEFVEINVRRSCFYPVETGIDYITVKGFEMAHAATPWAPPTADQPGLIGPNWSKGWIIEDNIIHDAKCSAISIGKEATTGNNYRSIRKDKPGYQYQLEAVFNAKRNGWSKEKIGSHIIRNNTIYDCGQNAIVGHLGGVFSEIYNNHIYNIALKREFYGHEIAGIKLHAAIDVQIHHNRIHDCSLGLWLDWEAQGTRVSKNLFYNNNRDVFVEVSHGPYLVDHNILSSEYAIDNMSQGGAYINNLIAGKMNQRKVLNRSTQYHLPHSTEVAGFAFVYGGDDRFYNNIFIGKEGLENVGTSHYNNCTTSLEEYIEKVNEVPGDLGEFERVEQPVYINKNAYFNGAEPFEKEKDNLVKKDFDPKLAIIDEGDEVYLSLQLPDEFENIVGDIHSTKTLERVRIVDAEYESPDGKELVLDTDYLDAKKPENSSIGPIALLKKGNNYIKVW

SEQ ID NO: 27 - protein, 804 aa, Aspegillis niger
MAHSMSRPVAATAAALLALALPQALAQANTSYVDYNIEANPDLYPLCIETIPLSFPDCQNGPLRSHLICDETATPYDRAASLISLFTLDELIANTGNTGLGVSRLGLPAYQVWSEALHGLDRANFSDSGAYNWATSFPQPILTTAALNRTLIHQIASIISTQGRAFNNAGRYGLDVYAPNINTFRHPVWGRGQETPGEDVSLAAVYAYEYITGIQGPDPESNLKLAATAKHYAGYDIENWHNHSRLGNDMNITQQDLSEYYTPQFHVAARDAKVQSVMCAYNAVNGVPACADSYFLQTLLRDTFGFVDHGYVSSDCDAAYNIYNPHGYASSQAAAAAEAILAGTDIDCGTTYQWHLNESIAAGDLSRDDIEQGVIRLYTTLVQAGYFDSNTTKANNPYRDLSWSDVLETDAWNISYQAATQGIVLLKNSNNVLPLTEKAYPPSNTTVALIGPWANATTQLLGNYYGNAPYMISPRAAFEEAGYKVNFAEGTGISSTSTSGFAAALSAAQSADVIIYAGGIDNTLEAEALDRESIAWPGNQLDLIQKLASAAGKKPLIVLQMGGGQVDSSSLKNNTNVSALLWGGYPGQSGGFALRDIITGKKNPAGRLVTTQYPASYAEEFPATDMNLRPEGDNPGQTYKWYTGEAVYEFGHGLFYTTFAESSSNTTTKEVKLNIQDILSQTHEDLASITQLPVLNFTANIRNTGKLESDYTAMVFANTSDAGPAPYPKKWLVGWDRLGEVKVGETRELRVPVEVGSFARVNEDGDWVVFPGTFELALNLERKVRVKVVLEGEEEVVLKWPGKE

SEQ ID NO: 28 - protein, 238 aa, Aspergillus aculeatus
MKLSLLSLATLASAASLQRRSDFCGQWDTATAGDFTLYNDLWGESAGTGSQCTGVDSYSGDTIAWHTSWSWSGGSSSVKSYVNAALTFTPTQLNCISSIPTTWKWSYSGSSIVADVAYDTFLAETASGSSKYEIMVWLAALGGAGPISSTGSTIATPTIAGVNWKLYSGPNGDTTVYSFVADSTTESFSGDLNDFFTYLVDNEGVSDELYLTTLEAGTEPFTGSNAKLTVSEYSISIE

SEQ ID NO: 29 - protein, 857 aa, Aspergillis niger
MRRTFAALVAGYLLDSVHAAASQAYTWKNVVTGGGGGFTPGIVFNPSAKGVAYARTDIGGAYRLNSDDTWTPLMDWANNSNWHDWGIDAIATDPVDTDRVYVAVGMYTNDWDPNDGSILRSTDQGDTWEETKLPFKVGGNMPGRGVGERLAVDPNDNSILYFGARSGNGLWKSTDYGETWSNVTAFKWTGTYFQDSSSTYTSDPVGIAWVTFDSTSGSSGSPTPRIFVGVVDTGESVFVSEDAGETWTWVSGEPMYGFLPHKGILSPSEHTLYISYSNGAGPYDGTNGTVHKYNITSGVWTDISPTSMTDTYYGYGGLAVDLQVPGTVMVAALNCWWPDELIWRSTDSGGTWSPIWAWNGYPSINYYYSYDISNAPWLQDDTSTDEFPVRVGWMVEALAIDPFDSDHWLYGTGETIYGGHDLQNWDSEHNVTIESLAVGIEEMAVLGLITPPGGPALLSAVGDDGGFYHTSLTTAPSQYYHTPTYSSTNGIDYAGNKPANIVRSGSSDSDPTLALSSSFGESWYADYAASSSTATGQVALSADADTILLMNSDGAYRSANSATLSAVSSLPSGAVIASDKANNTYFYGASGSSFYLSSDTAATFTVTTTLGSSTTANAIRAQPSLAGDVWVSTDTGLFHSTNYGKSFTQIGSGCTEGWSFGFGKPSSDGDYPVLFGFFTVDGVTGLFKTEDQGVNWQIISDAEHGFGSASANVVNGDLQNYGRVFVGTNGRGIFYGDPSGTLPSATATASSASSTAVKSSTSTSTSKVGSSTTVSSSTATTITTSSIKSTTLTTTTKSSSSTTSTSSTATGTASAYGQCGGSGFTGPTQCPSGWTCTYENEYYSQCKSIPGIATDRG

SEQ ID NO: 30 - protein, 523 aa, Irpex lacteus
MFRKAALLAFSFLAIAHGQQVGTNQAENHPSLPSQKCTASGCTTSSTSVVLDANWRWVHTTTGYTNCYTGQTWDASICPDGVTCAKACALDGADYSGTYGITTSGNALTLQFVKGTNVGSRVYLLQDASNYQMFQLINQEFTFDVDMSNLPCGLNGAVYLSQMDQDGGVSRFPTNTAGAKYGTGYCDSQCPRDIKFINGEANVEGWTGSSTDSNSGTGNYGTCCSEMDIWEANSVAAAYTPHPCSVNQQTRCTGADCGQGDDRYDGVCDPDGCDFNSFRMGDQTFLGKGLTVDTSRKFTIVTQFISDDGTTSGNLAEIRRFYVQDGNVIPNSKVSIAGIDAVNSITDDFCTQQKTAFGDTNRFAAQGGLKQMGAALKSGMVLALSLWDDHAANMLWLDSDYPTTADASNPGVARGTCPTTSGFPRDVESQSGSATVTYSNIKWGDLNSTFTGTLTTPSGSSSPSSPASTSGSSTSASSSASVPTQSGTVAQWAQCGGIGYSGATTCVSPYTCHVVNAYYSQCY

SEQ ID NO: 31 - protein, 984 aa, Thermobifida fusca
MRSLLSPRRWRTLASGALAAALAAAVLSPGVAHAAVACSVDYDDSNDWGSGFVAEVKVTNEGSDPIQNWQVGWTFPGNQQITNGWNGVFSQSGANVTVRYPDWNPNIAPGATISFGFQGTYSGSNDAPTSFTVNGVTCSGSQPANLPPDVTLTSPANNSTFLVNDPIELTAVASDPDGSIDRVEFAADNTVIGIDTTSPYSFTWTDAAAGSYSVTAIAYDDQGARTVSAPIAIRVLDRAAVIASPPTVRVPQGGTADFEVRLSNQPSGNVTVTVARTSGSSDLTVSSGSQLQFTSSNWNQPQKVTIASADNGGNLAEAVFTVSAPGHDSAEVTVREIDPNTSSYDQAFLEQYEKIKDPASGYFREFNGLLVPYHSVETMIVEAPDHGHQTTSEAFSYYLWLEAYYGRVTGDWKPLHDAWESMETFIIPGTKDQPTNSAYNPNSPATYIPEQPNADGYPSPLMNNVPVGQDPLAQELSSTYGTNEIYGMHWLLDVDNVYGFGFCGDGTDDAPAYINTYQRGARESVWETIPHPSCDDFTHGGPNGYLDLFTDDQNYAKQWRYTNAPDADARAVQVMFWAHEWAKEQGKENEIAGLMDKASKMGDYLRYAMFDKYFKKIGNCVGATSCPGGQGKDSAHYLLSWYYSWGGSLDTSSAWAWRIGSSSSHQGYQNVLAAYALSQVPELQPDSPTGVQDWATSFDRQLEFLQWLQSAEGGIAGGATNSWKGSYDTPPTGLSQFYGMYYDWQPVWNDPPSNNWFGFQVWNMERVAQLYYVTGDARAEAILDKWVPWAIQHTDVDADNGGQNFQVPSDLEWSGQPDTWTGTYTGNPNLHVQVVSYSQDVGVTAALAKTLMYYAKRSGDTTALATAEGLLDALLAHRDSIGIATPEQPSWDRLDDPWDGSEGLYVPPGWSGTMPNGDRIEPGATFLSIRSFYKNDPLWPQVEAHLNDPQNVPAPIVERHRFWAQVEIATAFAAHDELFGAGAP

SEQ ID NO: 32 - protein, 448 aa, Biospora sp.
MLFQVGTVLLLAWLSPTTDAAPGWGWRTVTVTETVTPSSTAAGTCSASSPSTSGIISSSTSSSTATISSASATSYPSTTYPTTPAYPICTSRAPFASIDDVHPRLFNYNGTGAKYFAGTNAWWTSYLMIDSDVNLVFSEIKNTQLQVVRIWGFGSVNTDPGPGTVFFQLLNSTGSYINYAANGIPRLDAVVSYAERNGVKIVLNFVNNWSALGGIASYNAAFGGNATSWYTDAESQKVYKDYIKLLVNRYKCSPAIFAWELANEPRCQGCDTSVIYNWATEVSQYIKSLDPRHMVALGDEGWFAPADGIGDGSYAYSGDQGVDFVKNLGIKTLDYGTFHLYPSSWGYNESWGSTWILQHNEVGAAHNKAVVLEEYGGPPTPNNHTAVEEPWQATVLKDTKLAMDQFWQFGTVLSTGLSDYDNFTIWYNSQEYVPLARDHAAAMLEKPV

SEQ ID NO: 33 - protein, 438 aa, Aspergillus fumigatus
MHPLPSVALLSAIGAVAAQVGPWGQCGGRSYTGETSCVSGWSCVLFNEWYSQCQPATTTSTSSVSATAAPSSTSSSKESVPSATTSKKPVPTGSSSFVKADGLKFNIDGETKYFAGTNAYWLPFLTNDADVDSVMDNLQKAGLKILRTWGFNDVNSKPSSGTVYFQLHDPSTGTTTINTGADGLQRLDYVVSAAEKRGIKLLIPLVNNWDDYGGMNAYVKAYGGSKTEWYTNSKIQSVYQAYIKAVVSRYRDSPAIMAWELSNEARCQGCSTDVIYNWTAKTSAYIKSLDPNHMVATGDEGMGVTVDSDGSYPYSTYEGSDFAKNLAAPDIDFGVFHLYTEDWGIKDNSWGNGWVTSHAKVCKAAGKPCLFEEYGLKDDHCSASLTWQKTSVSSGMAADLFWQYGQTLSTGPSPNDHFTIYYGTSDWQCGVADHLSTL

SEQ ID NO: 34 - protein, 937 aa, Aspergillus aculeatus
MRALPTTATTLLGVLFFPSASRSQYVRDLGTEQWTLSSATLNRTVPAQFPSQVHMDLLREGIIDEPYNDLNDFNLRWIADANWTYTSGKIEGLGEDYESTWLVFDGLDTFASISFCGQFVGATDNQFRQYMFDVSSILKACPEEPTLGIQFGSAPNIVDAIAQDPSSPTWPEGVQITYEYPNRWFMRKEQSDFGWDWGPAFAPAGPWKPGYVVQLKQAAPVYVRNTDLDIYRLGQINYLPPDQTQPWVVNASLDYLGSLPENPSMAIEVKDLQSGEILASRPLTNITVTEGSVTGVTVLEGVDPKLWWPQGLGDQNLYNVTISVTDGGNQSVAEVTKRTGFRTIFLNQRNITDAQLAQGIAPGANWHFEVNGHEFYAKGSNLIPPDCFWTRVTEDTMTRLFDAVVAGNQNMLRVWSSGAYLHDYIYDLADEKGILLCSEFQFSDALYPTDDAFLENVAAEVVYNVRRVNHHPSLALWAGGNEIESLMLLLVEAADPESYPFYVGEYEKMYISLFLPLVYENTRSISYSPSSTTEGYLDIDLSAPVPMAERYSNTTEGEYYGDTDHYNYDASIAFDYGTYPVGRFANEFGFHSMPSLQTWQQALTDPADLTFNSSVVMLRNHHYPAGGLMTDNYHNTVARHGRNDPGRAGLLPDAQHSVRPRGQLQRLVPRDPALPGGPLQVTNPVLPAGQRAARTPARVPVLAARGHLAGALVGGDRVRRPLEGPHYVARDIYKPVIVSPFWNYTTGALDIYVTSDLWTAAAGSVTLTWRDLSGKPIASNGGLPTKPLPFHVGALNSTRLYRMNMKQQPLPRHEDAILALELTATGSLPNTDEEVTFTHEQWFTPAFPKDLDLVNLRVRVEYDAPLGKFAVEATAGVALYTWLEHPEGVVGYFEENSFVVVPGQKKVVGFVVQADETDGEWVHDVTVRSLWDLNEGE

SEQ ID NO: 35 - protein, 1711 aa, Anaerocellum thermophilum
GSFNYGEALQKAIMFYEFQMSGKLPNWVRNNWRGDSALKDGQDNGLDLTGGWFDAGDHVKFNLPMSYTGTMLSWAVYEYKDAFVKSGQLEHILNQIEWVNDYFVKCHPSKYVYYYQVGDGSKDHAWWGPAEVMQMERPSFKVTQSSPGSTVVTETAASLAAASIVLKDRNPTKAATYLQHAKELYEFAEVTKSDAGYTAANGYYNSWSGFYDELSWAAVWLYLATNDSTYLTKAESYVQNWPKISGSNTIDYKWAHCWDDVHNGAALLLAKITGKDIYKQIIESHLDYWITGYNGERIKYTPKGLAWLDQWGSLRYATTTAFLAFVYSDWVGCPSTKKEIYRKFGESQIDYALGSAGRSFVVGFGTNPPKRPHHRTAHSSWADSQSIPSYHRHTLYGALVGGPGSDDSYTDDISNYVNNEVACDYNAGFVGALAKMYQLYGGNPIPDFKAIETPTNDEFFVEAGINASGTNFIEIKAIVNNQSGWPAKATDKLKFRYFVDLSELIKAGYSPNQLTLSTNYNQGAKVSGPYVWDASKNIYYILVDFTGTLIYPGGQDKYKKEVQFRIAAPQNVQWDNSNDYSFQDIKGVSSGSVVKTKYIPLYDGDVKVWGDGPGTSGATPTPTATATPTPTPTVTPTPTPTPTSTATPTPTPTPTVTPTPTPTPTATPTSTPTPTSTPSSTPVAGGQIKVLYANKETNSTTNTIRPWLKVVNTGSSSIDLSRVTIRYWYTVDGDKAQSAISDWAQIGASNVTFKFVKLSSSVSGADYYLEIGFKSGAGQLQAGKDTGEIQIRFNKSDWSNYNQGNDWSWMQSMTNYGENVKVTAYIDGVLVWGQEPSGATPTPTATPAPTVTPTPTPTPTSTPTATPTATPTPTPTPSSTPVAGGQIKVLYANKETNSTTNTIRPWLKVVNTGSSSIDLSRVTIRYWYTVDGDKAQSAISDWAQIGASNVTFKFVKLSSSVSGADYYLEIGFKSGAGQLQAGKDTGEIQIRFNKSDWSNYNQGNDWSWMQSMTNYGENVKVTAYIDGVLVWGQEPSGATPTPTATPAPTVTPTPTPTPTSTPTATPTATPTPTPTPSSTPSVVGEYGQRFMWLWNKIHDPANGYFNQDGIPYHSVETLICERPDYGHLTTSEAFSYYVWLEAVYGKLTGDWSKFKTAWDTLEKYMIPSAEDQPMRSYDPNKPATYAGEWETPDKYPSPLEFNVPVGKDPLHNELVSTYGSTLMYGMHWLMDVDNWYGYGKRGDGVSRASFINTFQRGPEESVWETVPHPSWEEFKWGGPNGFLDLFIKDQNYSKQWRYTDAPDADARAIQATYWAKVWAKEQGKFNEISSYVAKAARMGDYLRYAMFDKYFKPLGCQDKNAAGGTGYDSAHYLLSWYYAWGGALDGAWSWKIGSSHVHFGYQNPMAAWALANDSDMKPKSPNGASDWAKSLKRQIEFYRWLQSAEGAIAGGATNSWNGRYEKYPAGTATFYGMAYEPNPVYHDPGSNTWFGFQAWSMQRVVEYYYVTGDKDAGALLEKWVSWVKSVVKLNSDGTFAIPSTLDWKRQPDTWNGAYTGNSNLHVKVVDYGTDLGITASLANALLYYSAGTKKYGVFDEGAKNLAKELLDRMWKLYRDEKGLSAPEKRADYKRFFEQEVYIPAGWIGKMPNGDVIKSGVKFIDIRSKYKQDPDWPKLEAAYKSGQAPEFRYHRFWAQCDIAIANATYEILFGNQ

SEQ ID NO: 36 - protein, 1742 aa, Caldocellum saccharolyticum
MVVTFLFILGVVYGVKPWQEARAGSFNYGEALQKAIMFYEFQMSGKLPNWVRNNWRGDSALKDGQDNGLDLTGGWFDAGDHVKFNLPMSYTGTMLSWAAYEYKDAFVKSGQLEHILNQIEWVNDYFVKCHPSKYVYYYQVGDGGKDHAWWGPAEVMQMERPSFKVTQSSPGSAVVAETAASLAAASIVLKDRNPTKAATYLQHAKDLYEFAEVTKSDSGYTAANGYYNSWSGFYDELSWAAVWLYLATNDSTYLTKAESYVQNWPKISGSNIIDYKWAHCWDDVHNGAALLLAKITDKDTYKQIIESHLDYWTTGYNGERIKYTPKGLAWLDQWGSLRYATTTAFLAFVYSDWSGCPTGKKETYRKFGESQIDYALGSTGRSFVVGFGTNPPKRPHHRTAHSSWADSQSIPSYHRHTLYGALVGGPGSDDSYTDDISNYVNNEVACDYNAGFVGALAKMYLLYGGNPIPDFKAIETPTNDEFFVEAGINASGTNFIEIKAIVNNQSGWPARATNKLKFRYFVDLSELIKAGYSPNQLTLSTNYNQGAKVSGPYVWDSSRNIYYILVDFTGTLIYPGGQDKYKKEVQFRIAAPQNVQWDNSNDYSFQDIKGVSSGSVVKTKYIPLYDEDIKVWGEEPGTSGVSPTPTASVTPTPTPTPTATPTPTPTPTVTPTPTVTATPTPTPTPTSTPTVTPTPTPVSTPATSGQIKVLYANKETNSTTNTIRPWLKVVNSGSSSIDLSRVTIRYWYTVDGERAQSAISDWAQIGASNVTFKFVKLSSSVSGADYYLEIGFKSGAGQLQPGKDTGEIQIRFNKDDWSNYNQGNDWSWIQSMTSYGENEKVTAYIDGVLVWGQEPSGTTPAPTSTPTVTVTPTPTPTPTVTPTPTVTATPTPTPTPTSTPVSTPATGGQIKVLYANKETNSTTNTIRPWLKVVNSGSSSIDLSRVTIRYWYTVDGERAQSAISDWAQIGASNVTFKFVKLSSSVSGADYYLEIGFKSGAGQLQPGKDTGEIQIRFNKDDWSNYNQGNDWSWIQSMTSYGENEKVTAYIDGVLVWGQEPSGATPAPTVTPTPTVTPTPTPAPTPTATPTPTPTPTVTPTPTVAPTPTPSSTPSGLGKYGQRFMWLWNKIHDPASGYFNQDGIPYHSVETLICEAPDYGHLTTSEAFSYYVWLEAVYGKLTGDWSKFKTAWDTLEKYMIPSAEDQPMRSYDPNKPATYAGEWETPDKYPSPLEFNVPVGKDPLHNELVSTYGSTLMYGMHWLMDVDNWYGYGKRGDGVSRASFINTFQRGPEESVWETVPHPSWEEFKWGGPNGFLDLFIKDQNYSKQWRYTNAPDADARAIQATYWAKVWAKEQGKFNEISSYVGKAAKMGDYLRYAMFDKYFKPLGCQDKNAAGGTGYDSAHYLLSWYYAWGGALDGAWSWKIGCSHAHFGYQNPMAAWALANDSDMKPKSPNGASDWAKSLKRQIEFYRWLQSAEGAIAGGATNSWNGRYEKYPAGTATFYGMAYEPNPVYRDPGSNTWFGFQAWSMQRVAEYYYVTGDKDAGTLLEKWVSWIKSVVKLNSDGTFAIPSTLDWSGQPDTWNGTYTGNPNLHVKVVDYGTDLGITASLANALLYYSAGTKKYGVFDEEAKNLAKELLDRMWKLYRDEKGLSAPEKRADYKRFFEQEVYIPAGWTGKMPNGDVIKSGVKFIDIRSKYKQDPDWPKLEAAYKSGQVPEFRYHRFWAQCDIAIVNATYEILFGNQ

SEQ ID NO: 37 - protein, 274 aa, Thermotoga neapolitana
MRLVVSFLLVVSAFLFSAEVVLTDIGATDITFKGFPVTMELNFWNVKSYEGETWLKFDGEKVQFYADIYNIVLQNPDSWVHGYPEIYYGYKPWAAHNSGTEILPVKVKDLPDFYVTLDYSIWYENDLPINLAMETWITRKPDQTSVSSGDVEIMVWFYNNILMPGGQKVDEFTTTIEINGSPVETKWDVYFAPWGWDYLAFRLTTPMKDGRVKFNVKDFVEKAAEVIKKHSTRVENFDEMYFCVWEIGTEFGDPNTTAAKFGWTFKDFSVEIGE

SEQ ID NO: 38 - protein, 319 aa, Thermotoga neapolitana
MSKKKFVIVSILTILLVQAIYFVEKYHTSEDKSTSNTSSTPPQTTLSTTKVLKIRYPDDGEWPGAPIDKDGDGNPEFYIEINLWNILNATGFAEMTYNLTSGVLHYVQQLDNIVLRDRSNWVHGYPEIFYGNKPWNANYATDGPIPLPSKVSNLTDFYLTISYKLEPKNGLPINFAIESWLTREAWRTTGINSDEQEVMIWIYYDGLQPAGSKVKEIVVPIIVNGTPVNATFEVWKANIGWEYVAFRIKTPIKEGTVTIPYGAFISVAANISSLPNYTELYLEDVEIGTEFGTPSTTSAHLEWWITNITLTPLDRPLIS

SEQ ID NO: 39 - protein, 274 aa, Pyrococcus furiosus; identical to SEQ ID NO: 37

SEQ ID NO: 40 - protein, 260 aa, Rhodothermus marinus
MNVMRAVLVLSLLLLFGCDWLFPDGDNGKEPEPEPEPTVELCGRWDARDVAGGRYRVINNVWGAETAQCIEVGLETGNFTITRADHDNGNNVAAYPAIYFGCHWAPARAIRDCAARAGAVRRAHELDVTPITTGRWNAAYDIWFSPVTNSGNGYSGGAELMIWLNWNGGVMPGGSRVATVELAGATWEVWYADWDWNYIAYRRTTPTTSVSELDLKAFIDDAVARGYIRPEWYLHAVETGFELWEGGAGLRTADFSVTVQ

SEQ ID NO: 41 - protein, 453 aa, Streptomyces sp. M23
MSRSRTAMLAALTLAAGSMTLALAAGPASAGPAAPTARVDNPYVGATMYVNPEWSALAASEPGGDRVADQPTAVWLDRIATIEGVDGKMGLREHLDEALQQKGSGELVVQLVIYDLPGRDCAALASNGELGPDELDRYKSEYIDPIADILSDSKYEGLRIVTVIEPDSLPNLVTNAGGTDTTTEACTTMKANGNYEKGVSYALSKLGAIPNVYNYIDAAHHGWLGWDTNLGPSVQEFYKVATSNGASVDDVAGFAVNTANYSPTVEPYFTVSDTVNGQTVRQSKWVDWNQYVDEQSYAQALRNEAVAAGFNSDIGVIIDTSRNGWGGSDRPSGPGPQTSVDAYVDGSRIDRRVHVGNWCNQSGAGLGERPTAAPASGIDAYTWIKPPGESDGNSAPVDNDEGKGFDQMCDPSYQGNARNGYNPSGALPDAPLSGQWFSAQFRELMQNAYPPLS

SEQ ID NO: 42 - protein, 457 aa, Thermoascus aurantiacus
MYQRALLFSFFLAAARAQQAGTVTAENHPSLTWQQCSSGGSCTTQNGKVVIDANWRWVHTTSGYTNCYTGNTWDTSICPDDVTCAQNCALDGADYSGTYGVTTSGNALRLNFVTQSSGKNIGSRLYLLQDDTTYQIFKLLGQEFTFDVDVSNLPCGLNGALYFVAMDADGGLSKYPGNKAGAKYGTGYCDSQCPRDLKFINGQANVEGWQPSANDPNAGVGNHGSCCAEMDVWEANSISTAVTPHPCDTPGQTMCQGDDCGGTYSSTRYAGTCDPDGCDFNPYRQGNHSFYGPGQIVDTSSKFTVVTQFITDDGTPSGTLTEIKRFYVQNGKVIPQSESTISGVTGNSITTEYCTAQKAAFGDNTGFFTHGGLQKISQALAQGMVLVMSLWDDHAANMLWLDSTYPTDADPDTPGVARGTCPTTSGVPADVESQYPNSYVIYSNIKVGPINSTFTAN

SEQ ID NO: 43 - protein, 660 aa, Clostridium thermocellum
MGQKHFKRSLLSVLTISALIISCLFSFIFVNADDTSEEPALEGLSIHYMDGTLDVKYQSMRPYIIIHNNSGMDVDMADLRVRYYYEKEGVTEEVLTCFYTAIGADKIFAEFHPELGYAEIGFTSDAGIIKSGGNSGQLQLVLKKISNGYYDQSNDYSYDPSYTDYAEYDKITLYYKGKLVWGKEGPPPPPEPTPPPNNDDWLHVEGNLIKDAQGNTVYLTGINWFGFETDGANGFHGLNKCNLEDSLDLMAKLGFNILRIPISAEIILQWKNGERVETSFVNTYENPRLDGLSSLEILDYTINHMKKNGMKAMIDMHSSTKDSYQENLWYNKDITMEEFIEAWKWIVERYKDDDTVIAVDLKNEPHGKYSGPNIAKWDDSNDPNNWKRAAEIIAEEILAINPNLLIVVEGVEAYPMEGYDYTNCGEFTTYCNWWGGNLRGVADHPVVISAPDKLVYSVHDYGPDIYMQPWFKKDFDINTLYEECWYPNWYYIVEQNIAPMLIGEWGGKLINENNRKWLECLATFIAEKKLHHTFWAFNPNSADTGGLMLEDWKTVDEEKYAIIVPTLWKKGLDHVIPLGGITEDTFKYGDVNGDFAVNSNDLTLIKRYVLKNIDEFPSPHGLKAADVDGNEKITSSDAALVKRYVLRAITSFPVEENQNE

SEQ ID NO: 44 - protein, 733 aa, Sporotrichum thermophile
MTLQAFALLAAAALVRGETPTKVPRDAPRGAAAWEAAHSSAAAALGKLSQQDKINIVTGVGWNKGPCVGNTPAISSINYPQLCLQDGPLGVRFGSSITAFTPGIQAASTWDVDLIRQRGEYMGAEFKGCGIHVQLGPVAGPLGKVPQGGRNWEGFGVDPYLTGIAMAETIEGIQSAGVQATAKHYILNEQELNRETMSSNVDDRTLHELYLWPFADAVHSNVASVMCSYNKINGTWACENDRVLNVILKQELGFPGYVMSDWNAQHSTDDAANHGMDMTMPGSDFNGGTILWGPQLDSAVNSGRVPKSRLDDMVERILAAWYLLGQDSNYPAINIGANVQGNHKENVRAVARDGIVLLKNDDGILPLKKPAKLALIGSAAVVNPQGLNSCQDQGCNKGALGMGWGSGAVNYPYFVAPYDALKARAQEDGTTVSLHNSDSTSGVANVASDADAAIVVITADSGEGYITVEGAAGDRLNLDPWHNGNELVKAVAAANKNTIVVVHSVGPIILETILATEGVKAIVWAGLPSQENGNALVDILYGLASPSGKLVYTIAKREQDYGTAVVRGDDTFPEGLFVDYRHFDKENIEPRYEFGFGLSYTNFTYADLELTSTATAGPATGETIPGGAADLWEEVATVTATITNSGGVDGAEVAQLYLTLPSSAPATPPKQLRGFAKLKLAAGASGTATFSLRRRDLSYWDTGRGQWVVPEGEFGVSVGASSRDIRLTGSFRV

SEQ ID NO: 45 - protein, 866 aa, Periconia sp
MASWLAPALLAVGLASAQAPFPNGSSPLNDITSPPFYPSPWMDPSAAGWAEAYTKAQAFVRQLTLLEKVNLTTGVGWEGEACVGNTGSIPRLGFPGFCTQDSPLGVRFADYVSAFTAGGTIAASWDRSEFYRRGYQMGVEHRGKGVDVQLGPVVGPIGRHPKGGRNWEGFSPDPVLSGIAVAETVKGIQDAGVIACTKHFILNEQEHFRQPGNVGDFGFVDAVSANLADKTLHELYLWPFADAVRAGTGSIMCSYNKANNSQVCQNSYLQNYILKGELGFQGFIMSDWDAQHSGVASTLAGLDMTMPGDTDFDSGFSFWGPNMTLSIINGTVPEWRLDDAATRIMAAYYLVGRDRHAVPVNFNSWSKDTYGYQHAYAKVGYGLINQHVDVRADHFKSIRTAAAKSTVLLKNNGVLPLKGTEKYTAVFGNDAGEAQYGPNGCADHGCDNGTLAMGWGSGTADYPYLVTPLEAIKRTVGDHGGVIASVTDNYAFSQIMALAKQATHAIVFVNADSGEGYITVDGNEGDRNNLTLWQNGEELVRNVSGYCNNTIVVIHSVGPVLVDSFNNSPNVSAILWAGLPGQESGNAITDVLYGRVNPGGKLPFTIGKSAEEYGPDIIYEPTAGHGSPQANFEEGVFIDYRSFDKKNITPVYEFGFGLSYTNFSYSNLVVTRVNAPAYVPTTGNTTAAPTLGNSSKDASDYQWPANLTYVNKYIYPYLNSTDLKEASNDPEYGIEHEYPEGATDGSPQPRIAAGGGPGGNPQLWDVLYKVTATVTNNGAVAGDEVAQLYVSLGGPEDPPVVLRNFDRLTIAPGQSVEFTADITRRDVSNWDTVSQNWVISNSTKTVYVGASSRKLPLKATLPSSSY

SEQ ID NO: 46 - protein, 862 aa, Volvariella volvacea
MPPSDFAKANIDEIVEQLTLDEAISLTAGVGFWHTHAIERLGVPAVKVSDGPNGIRGNHFFMGTPAKCLPSSTALGATWDPEVVEEVGLKLLAPEAKLRAASLVLAPTSNIQRNPLGGRSFESFSEDPYLSGIISASYVNGVQKGGIGATIKHFVGNDKEDDRQGYDSIISERALREIYLLPFMLTQKYAAPWAIMTAYNRVNGVHVAEDPFLLKQVLRNEWKYKGLIMSDWFGMYSVDHGIKAGLDLEMPGINKWRTLDLVNRTIQARKLTPRDIKDRARVVLELVKKCAQGAPEILDGDGEERTVELESDKLLMRRIASESIVLLKNDNVLPLKPEGGAIKKIAVVGGNAKAQVLSGGGSAALKASYFISPYDGIKAALEPHGVEVTFSEGARAYKTLPTLEWDLETETGERGWIGTWHTHESDDSMTALDQPFIAPRLVDETRIFISTSYPKGITKRWTMRLKGYLKPREKDTNFEFGLIAAGRAKLWVDGQLVIDNWTRQRRGEAFFGSGSQEETGVYLLKAGKKHEIYVEYCNVRAPADGDEDEAIMDSNPGVRLGGAEVANADDLLSEAVKLASEADAVIAVVGLNADWETEGNDRRTLALPGRTDELVEKVAKVNSKTVVVTQAGSAITLPWLDSVAAVVHAWYLGNATGDAIADVLFGKQNPSGKLSLTFPKRLEDVPSHGHFGSENGKVRYAEDLFVGYKHYHHRNIEPLFPFGFGLSYTTFSLSNLQLSAPVIDHATSSFSLTATLSITNTGPVTGSEVAQLYVSYPETSELTHAPLQLRAFKKVKDLQPGETREVKLELDKYAVSYWNDRYQTWAVENGEYEIKVGNSSVSKDLTLVQRFVVKDGFEWRGI

SEQ ID NO: 47 - DNA, 495 bp, artificial sequence
  synthetic
atgaatacaggcactatcaactttaacggaaagattacttccgcgacgtgcacaatcgaccccgaggtgaacggaaatcgcacatccactatcgacctgggccaggccgcgatcagtggacacggcacggttgtagactttaagctcaagccagcccctggctctaacgactgcttggccaagacaaacgctcggattgactggtcgggctcgatgaactcgcttggattcaataacactgctagcggcaataccgctgccaaagggtatcacatgaccctacgtgcgactaacgtgggaaacggtagtggtggtgcgaacatcaacacttcattcaccacggcggaatacacccacacttcggctatacagtccttcaactattccgcccaacttaagaaagacgatagggcaccttctaacggagggtataaggcgggagtcttcacgaccagcgcgtcattcctcgtgacctatatgaaggacgagctctag

SEQ ID NO: 48 - DNA, 798 bp, artificial sequence
  synthetic
atggacaagcgcctcttcatctcacacgtgatcctcatcttcgctcttatcctcgtgatctcaactccaaacgtgcttgctgagtcacagccagaccccaagccagacgagttgcacaagtcatctaagttcactggccttatggagaacatgaaggtgctttacgacgacaaccacgtgtctgctatcaacgtgaagtcaatcgaccagttcagatacttcgacctcatctactctatcaaggacacaaagctcggcaactacgacaacgtgagggtggagttcaagaacaaggaccttgctgacaagtacaaggacaagtacgtggacgtgttcggcgccaacgcttactaccagtgcgctttctctaagaagaccaacgacatcaactctcaccagacagacaagaggaagacatgcatgtacggcggcgtgactgagcacaacggaaaccagcttgacaagtacaggtctatcaccgtgagggtgttcgaggacggaaagaaccttctttctttcgacgtgcagacaaacaagaagaaggtgaccgcccaggagctggactaccttaccaggcactaccttgtgaagaacaagaagctctacgagttcaacaactcaccatacgagaccggatacatcaagttcatcgagaacgagaactctttctggtacgacatgatgcccgcccctggtgacaagttcgaccagtctaagtaccttatgatgtacaacgacaacaagatggtggactctaaggacgtgaagatcgaggtgtaccttactactaagaagaag

SEQ ID NO: 49 - DNA, 810 bp, artificial sequence
  synthetic
atggacaagcgcctcttcatctcacacgtgatcctcatcttcgctcttatcctcgtgatctcaactccaaacgtgcttgctgagtcacagccagaccccaagccagacgagttgcacaagtcatctaagttcactggccttatggagaacatgaaggtgctttacgacgacaaccacgtgtctgctatcaacgtgaagtcaatcgaccagttcagatacttcgacctcatctactctatcaaggacacaaagctcggcaactacgacaacgtgagggtggagttcaagaacaaggaccttgctgacaagtacaaggacaagtacgtggacgtgttcggcgccaacgcttactaccagtgcgctttctctaagaagaccaacgacatcaactctcaccagacagacaagaggaagacatgcatgtacggcggcgtgactgagcacaacggaaaccagcttgacaagtacaggtctatcaccgtgagggtgttcgaggacggaaagaaccttctttctttcgacgtgcagacaaacaagaagaaggtgaccgcccaggagctggactaccttaccaggcactaccttgtgaagaacaagaagctctacgagttcaacaactcaccatacgagaccggatacatcaagttcatcgagaacgagaactctttctggtacgacatgatgcccgcccctggtgacaagttcgaccagtctaagtaccttatgatgtacaacgacaacaagatggtggactctaaggacgtgaagatcgaggtgtaccttactactaagaagaagaaagatgagttg

SEQ ID NO: 50 - DNA, 786 bp, artificial sequence
  synthetic
atgatggcaaagctcgtgttctctctttgcttccttcttttctccggatgctgcttcgctttctccatggagtcacagccagaccccaagccagacgagttgcacaagtcatctaagttcactggccttatggagaacatgaaggtgctttacgacgacaaccacgtgtctgctatcaacgtgaagtcaatcgaccagttcagatacttcgacctcatctactctatcaaggacacaaagctcggcaactacgacaacgtgagggtggagttcaagaacaaggaccttgctgacaagtacaaggacaagtacgtggacgtgttcggcgccaacgcttactaccagtgcgctttctctaagaagaccaacgacatcaactctcaccagacagacaagaggaagacatgcatgtacggcggcgtgactgagcacaacggaaaccagcttgacaagtacaggtctatcaccgtgagggtgttcgaggacggaaagaaccttctttctttcgacgtgcagacaaacaagaagaaggtgaccgcccaggagctggactaccttaccaggcactaccttgtgaagaacaagaagctctacgagttcaacaactcaccatacgagaccggatacatcaagttcatcgagaacgagaactctttctggtacgacatgatgcccgcccctggtgacaagttcgaccagtctaagtaccttatgatgtacaacgacaacaagatggtggactctaaggacgtgaagatcgaggtgtaccttactactaagaagaag

SEQ ID NO: 51 - DNA, 810 bp, artificial sequence
  synthetic
atgatggcaaagctcgtgttctctctttgcttccttcttttctccggatgctgcttcgctttctccatggagtcacagccagaccccaagccagacgagttgcacaagtcatctaagttcactggccttatggagaacatgaaggtgctttacgacgacaaccacgtgtctgctatcaacgtgaagtcaatcgaccagttcagatacttcgacctcatctactctatcaaggacacaaagctcggcaactacgacaacgtgagggtggagttcaagaacaaggaccttgctgacaagtacaaggacaagtacgtggacgtgttcggcgccaacgcttactaccagtgcgctttctctaagaagaccaacgacatcaactctcaccagacagacaagaggaagacatgcatgtacggcggcgtgactgagcacaacggaaaccagcttgacaagtacaggtctatcaccgtgagggtgttcgaggacggaaagaaccttctttctttcgacgtgcagacaaacaagaagaaggtgaccgcccaggagctggactaccttaccaggcactaccttgtgaagaacaagaagctctacgagttcaacaactcaccatacgagaccggatacatcaagttcatcgagaacgagaactctttctggtacgacatgatgcccgcccctggtgacaagttcgaccagtctaagtaccttatgatgtacaacgacaacaagatggtggactctaaggacgtgaagatcgaggtgtaccttactactaagaagaagggtggacaccatcaccatcaccat

SEQ ID NO: 52 - protein, 20 aa, Human
MEPWPLLLLFSLCSAGLVLG

SEQ ID NO: 53 - protein, 27 aa, S. aureus
MDKRLFISHVILIFALILVISTPNVLA

SEQ ID NO: 54 - protein, 33 aa, A. thaliana
MPPQKENHRTLNKMKTNLFLFLIFSLLLSLSSA

SEQ ID NO: 55 - protein, 37 aa, A. thaliana
MPPQKENHRTLNKMKTNLFLFLIFSLLLSLSSAKDEL

SEQ ID NO: 56 - protein, 19 aa, Human
MALVLEIFTLLASICWVSA

SEQ ID NO: 57 - protein, 27 aa, Staphylococcus aureus
MDKRLFISHVILIFALILVISTPNVLA

SEQ ID NO: 58 - protein, 22 aa, Glycine max
MAKLVFSLCFLLFSGCCFAFSM

SEQ ID NO: 59 - protein, 33 aa, Arabidopsis thaliana; identical to SEQ ID NO: 54

SEQ ID NO: 60 - protein, 20 aa, Trametes versicolor
MGLQRFSFFVTLALVARSLA

SEQ ID NO: 61 - protein, 22 aa, Talaromyces emmersonii
MARFSILSTIYLYILFIGSCLA

SEQ ID NO: 62 - protein, 17 aa, Myceliophthora thermophile
MTLQAFALLAAAALVRG

SEQ ID NO: 63 - protein, 519 aa, Trametes versicolor; identical to SEQ ID NO: 1

SEQ ID NO: 64 - protein, 300 aa, Talaromyces emmersonii; identical to SEQ ID NO: 20

SEQ ID NO: 65 - protein, 733 aa, Sporotrichum thermophile; identical to SEQ ID NO: 44